(19) United States
(12) Patent Application Publication
(10) Pub. No.: US 2026/0084134 A1
(43) Pub. Date: Mar. 26, 2026

(54) STORAGE AND DELIVERY OF GASEOUS CHEMICAL REACTANTS IN CHEMICAL PROCESSES USING METAL-ORGANIC FRAMEWORKS

(71) Applicant: Cornell University, Ithaca, NY (US)

(72) Inventors: Phillip MILNER, Ithaca, NY (US); Kaitlyn KEASLER, Ithaca, NY (US); Mary ZICK, Circleville, NY (US)

(21) Appl. No.: 18/858,385

(22) PCT Filed: Apr. 21, 2023
(86) PCT No.: PCT/US2023/019456

§ 371 Date: Oct. 21, 2024

Related U.S. Application Data
(60) Provisional application No. 63/333,435, filed on Apr. 21, 2022.

(51) Int. Cl. B01J 20/22; B01J 20/30; B01J 20/34; C07C 45/50; C07F 3/02

(52) U.S. Cl. CPC B01J 20/226; B01J 20/3085; B01J 20/3425; B01J 20/3483; C07C 45/50; C07F 3/02

(57) ABSTRACT

Methods of providing one or more gaseous chemical reactant(s) in a chemical process and methods of making metal-organic frameworks (MOF(s)). In various examples, a method comprises forming a chemical reaction mixture comprising: MOF(s) comprising one or more gaseous chemical reactant(s) (gas-loaded MOF(s)) and optionally, additional chemical reactant(s). where a chemical reaction of a chemical process occurs between at least a portion of the gaseous chemical reactant(s) and at least a portion of the additional chemical reactant(s), if present. In various examples, each individual MOF comprises the following structure and/or formula: Mₙ(polycarboxylate)ₘ. In various examples, the MOF(s) are sequestered in an inert material or are present in an inert container configured to release at least a portion or all of the MOF(s) into the reaction mixture. In various examples, the gaseous chemical reactant(s) is/are hazardous gaseous chemical reactant(s), sensitive gaseous chemical reactant(s), environmentally harmful gaseous chemical reactant(s), or any combination thereof.

### CROSS-REFERENCE TO RELATED APPLICATIONS

This application claims the benefit of U.S. Provisional Patent Application No. 63/333,435, filed Apr. 21, 2022; the contents of the above-identified application are hereby fully incorporated herein by reference in their entirety.

### STATEMENT REGARDING FEDERALLY SPONSORED RESEARCH

This invention was made with government support under grant number GM138165 awarded by the National Institutes of Health. The government has certain rights in the invention.

### BACKGROUND OF THE DISCLOSURE

Fluorine is ubiquitous in the pharmaceutical and agrochemical industries because it improves the bioavailability and metabolic stability of molecules. However, most modern fluoroalkylation and fluorovinylation protocols rely on reagents that are expensive, explosive, or otherwise challenging to use. Fluorinated gaseous reagents are promising alternatives that are overlooked for late-stage functionalization because they require specialized equipment. Fluorinated organic molecules account for 20-30% of active pharmaceutical ingredients (APIs) and >40% of agrochemicals due to their improved metabolic stabilities and membrane permeabilities compared to their non-fluorinated analogues. In addition, ¹⁸F-labeled compounds are prominent radiotracers for positron emission tomography (PET). Despite the importance of fluorine across numerous fields, the selective, late-stage introduction of fluorine and fluoroalkyl/fluorovinyl groups into drug-like molecules remains a frontier in organic synthesis. For example, many common fluorination reagents are expensive, highly reactive, and, in some cases, explosive. Simple fluorinated commodity chemicals such as vinylidene fluoride (VDF), trifluoropropene (TFP), hexafluoropropene (HFP), and trifluoromethyl iodide (TFMI) represent valuable and inexpensive potential building blocks for the installation of fluoroalkyl and fluorovinyl groups. For example, VDF and TFP provide natural entry points for the synthesis of fluorinated alkenes, which are important bioisosteres in medicinal chemistry and remain synthetically challenging to access. However, these reagents remain underexplored because they are gases at room temperature and pressure (RTP). The use of gaseous reagents necessitates specialized equipment for safe handling. Fluorinated gases are also generally toxic, flammable, ozone-depleting, and/or environmentally destructive, making them challenging to employ for high-throughput reaction discovery. As such, a general strategy for safely using fluorinated gases would greatly facilitate the synthesis of fluorinated molecules relevant to medicinal chemistry, agriculture, biomedical imaging, and beyond.

The use of gaseous reagents in organic synthesis generally requires handling the gas directly (e.g., filling a balloon from a cylinder) or generating the gas in/ex situ from stable molecular precursors. Both strategies suffer from key limitations. The former is simple, but it is low-throughput, lacks stoichiometric control, and produces significant gas waste—an issue that is exacerbated with toxic and environmentally destructive gases. Although the latter approach is amenable to high-throughput screening, it requires the design of a new delivery strategy for each gas and results in significant chemical waste.

### SUMMARY OF THE DISCLOSURE

The present disclosure provides, inter alia, methods of providing one or more gaseous chemical reactant(s) in a chemical process. The present disclosure also provides methods of making metal-organic framework(s) (MOF(s)).

In various examples, a method of providing one or more gaseous chemical reactant(s) in a chemical process comprises: forming a chemical reaction mixture comprising: one or more MOF(s) comprising one or more gaseous chemical reactant(s), where each individual MOF comprises the following structure and/or formula: Mₙ(polycarboxylate)ₘ(e.g., where M is a metal ion, n is 1, 2, 3, or the like, and m is 1, 2, 3, or the like), and the gaseous chemical reactant(s) is/are material(s) which exist in a gas phase under the reaction conditions of the chemical reaction mixture; and optionally, one or more additional chemical reactant(s), where a chemical reaction of the chemical process occurs between at least a portion of, substantially all of, or all of the gaseous chemical reactant(s) and at least a portion of, substantially all of, or all of the additional chemical reactant(s), if present. In various examples, M is independently at each occurrence chosen from magnesium (Mg) ions, nickel (Ni) ions, cobalt (Co) ions, copper (Cu) ions, iron (Fe) ions, manganese (Mₙ) ions, cadmium (Cd) ions, zinc (Zn) ions, aluminum (Al) ions, zinc (Zr) ions, and the like. In various examples, the polycarboxylate(s) is/are independently at each occurrence chosen from 2,5-dioxido-1,4-benzenedicarboxylate (dobdc⁴⁻), 2,4-dioxidobenzene-1,3-dicarboxylate (m-dobdc⁴⁻), benzene-1,3,5-tricarboxylate (btc³⁻), and 1,4-benzenedicarboxylate (bdc²⁻), and 2-amino-1,4-benzenedicarboxylate (NH₂-bdc²⁻). In various examples, the ratio of n/m is from about 1:3 to about 3:1, or the like. In various examples, the MOF(s), independently at each occurrence, each comprise the following structure and/or formula: Mg₂(dobdc), Mn₂(dobdc), Fe₂(dobdc), Co₂(dobdc), Ni₂(dobdc), Cu₂(dobdc), Zn₂(dobdc), Mg₂(m-dobdc), Ni₂(m-dobdc), Cu₃(btc)₂, Fe₃O(OH)(btc)₂, Al₃O(OH)(NH₂-bdc)₃, or the like.

In various examples, at least a portion or all of the MOF(s) are sequestered in an inert material configured to expose at least a portion of, substantially all, or all the MOFs to the reaction mixture under the reaction conditions or at least a portion or all of the MOF(s) are present in an inert container configured to release at least a portion of, substantially all, or all the MOF(s) into the reaction mixture. In various examples, the gaseous chemical reactant(s) is/are independently at each occurrence chosen from hazardous gaseous chemical reactant(s), sensitive gaseous chemical reactant(s), environmentally harmful gaseous chemical reactant(s), and any combination thereof. In various examples, the gaseous chemical reactant(s) is/are chosen from halogenated gaseous chemical reactant(s), oxocarbon gaseous chemical reactant(s), halogen gaseous chemical reactant(s), sulfur gaseous chemical reactant(s), and any combination thereof. In various examples, the additional chemical reactant(s) is/are chosen from Negishi coupling reactant(s), Heck coupling reactant(s), trifluoromethylation reactant(s), defluorinative cross-coupling reactant(s), carbonylative Suzuki coupling reactant(s), difluoromethylation reactant(s), copper-catalyzed borylation reactant(s), hydroarylation reactant(s), aminocarbonylation reactant(s), olefin metathesis reactant(s), fluoroalkylation reactant(s), deoxyfluorination reactant(s), deoxyfluoroalkoxylation reactant(s), fluoroalkylthiolation reactant(s), fluoroalkylselenation reactant(s), fluorovinylation reactant(s), fluoroalkynylation reactant(s), pentafluorosulfanylation reactant(s), and the like. In various examples, the chemical reaction mixture comprises one or more solvent(s) or the like. In various examples, the gas-loaded MOF(s), at a temperature of about −196° C. to about 55° C., including all 0.1° C. values and ranges therebetween, comprise(s), on average, from about 0.1 millimole (mmol) gaseous chemical reactant(s)/gram gas-loaded MOF(s) to about 10 mmol gaseous chemical reactant(s)/gram gas-loaded MOF(s), including all 0.01 mmol values and ranges therebetween. In various examples, the method further comprising, prior to the occurrence of the chemical reaction, releasing at least a portion of, substantially all of, or all of the gaseous chemical reactant(s) from the gas-loaded MOF(s) into the chemical reaction mixture. In various examples, the releasing at least a portion of, substantially all of, or all of the gaseous chemical reactant(s) from the gas-loaded MOF(s) into the chemical reaction mixture is achieved by increasing the chemical reaction mixture temperature, reducing the chemical reaction mixture pressure, adding a coordinating solvent to displace the gas, irradiation of the reaction mixture with light, sonication of the reaction mixture, mechanical grinding of the reaction mixture, or the like, or any combination thereof. In various examples, the method further comprising, prior to forming the chemical reaction mixture: forming the gas-loaded MOF(s); and optionally, maintaining the gas-loaded MOF(s) under inert and/or anhydrous conditions. In various examples, the forming the gas-loaded MOF(s) comprises: optionally, activating the MOF(s); forming a MOF gas-loading reaction mixture comprising: the gaseous chemical reactant(s); and the MOF(s), where the gas-loaded MOF(s) is/are formed, and optionally, isolating and/or activating the gas-loaded MOF(s). In various examples, the method further comprising, prior to forming the gas-loaded MOF(s), forming the MOF(s). In various examples, the method further comprising, after the gas-loaded MOF(s) has/have released a portion of, substantially all of, or all of the gaseous chemical reactant(s) into the chemical reaction mixture and/or has/have reacted with at least a portion of, substantially all of, or all of the additional chemical reactant(s) in the chemical reaction mixture, where spent gas-loaded MOF(s) are formed, isolating and/or activating the spent gas-loaded MOF(s), where recycled MOF(s) is/are formed.

In various examples, a method of making MOF(s), where each individual MOF comprises (or has) the following formula and/or structure: Mₙ(polycarboxylate)ₘ, where M is a metal ion independently at each occurrence chosen from Mg ions, Mₙ ions, Co ions, Ni ions, Cu ions, Zn ions, Fe ions, Al ions and the like, n is 1, 2, 3, or the like, and m is 1, 2, 3 or the like, comprises: forming a first MOF reaction mixture comprising: one or more compound(s) independently comprising one or more of the metal ion(s); one or more polycarboxylic acid(s) and/or salt(s) thereof; and one or more basic solvent(s), where the reaction mixture comprises 0.1 mol/liter (M) or greater of: the compound(s) comprising the metal ion(s); the polycarboxylic acid(s) and/or the salt(s) thereof; or both; and heating the reaction mixture, where one or more first solid(s) is/are formed; optionally, isolating the first solid(s); forming a second MOF reaction mixture comprising: the first solid(s); and one or more basic solvent(s), optionally, where the second MOF reaction mixture is maintained under inert and/or anhydrous conditions, the one or more MOF(s) is/are formed; and optionally, isolating and/or activating the MOF(s). In various examples, the metal compound(s) comprise magnesium salt(s), nickel salt(s), manganese salt(s), iron salt(s), cobalt salt(s), copper salt(s), zinc salt(s), aluminum salt(s), or the like, or a hydrate thereof, or any combination thereof. In various examples, the polycarboxylic acid(s) is/are independently at each occurrence chosen from 2,5-dihydroxyterephthalic acid (H₄dobdc), 1,3,5-benzenetricarboxylic acid (H₃btc), 4,6-dihydroxyisophthalic acid (m-H₄dobdc), 2-aminoterephthalic acid (NH₂—H₂bdc), and partially or completely deprotonated structural analogs thereof, and the like. In various examples, the first MOF reaction mixture comprise from about 1 equivalent(s) (eq) to about 10 eq, including all 0.1 eq values and ranges therebetween, polycarboxylic acid(spolycarboxylate salt(s), or any combination thereof. In various examples, the first MOF reaction mixture is held at a temperature of from about −150 degrees Celsius (° C.) to about 200° C., including all 0.1° C. values and ranges therebetween.

### BRIEF DESCRIPTION OF THE FIGURES

For a fuller understanding of the nature and objects of the disclosure, reference should be made to the following detailed description taken in conjunction with the accompanying figures.

FIGS. 1A-C shows reactions with VDF. NMR yields were determined by ¹⁹F NMR spectroscopy using fluorobenzene as an internal standard. Isolated yields are shown in parentheses. See supplementary information for experimental details. (A) A sample of VDF-Mg₂(dobdc) in a N₂-filled glovebox was transferred to a custom-built, air-free solid-addition funnel for delivery on the benchtop. The percentage of VDF delivered to a solution of THF/Et₂O over time is shown, assuming a storage capacity of 7.95 mmol VDF per 1.00 g of Mg₂(dobdc). (B) Scope of Pd-catalyzed defluorinative coupling of VDF and (hetero)arylboronic acids using VDF-Mg₂(dobdc). (C) Scope of Negishi coupling of (hetero)aryl halides and VDF-ZnCl•TMEDA synthesized from VDF-Mg₂(dobdc). *Pd(PPh₃)₄, †8 h, 10 mol % XPhos Pd G3, ‡10 mol % Xphos Pd G3, ¶18 h, 10 mol % Xphos Pd G3, § 11 h, 10 mol % Xphos Pd G3. DMF: N,N-dimethylformamide, TFA: trifluoroacetate, dtbbpy: 4,4′-di-tert-butyl-2,2′-dipyridyl, Ph: phenyl, THF: tetrahydrofuran, Et₂O: diethyl ether, Otf: trifluoromethanesulfonate, TMEDA: tetramethylethylenediamine, Me: methyl, Ts: p-toluenesulfonyl.

FIGS. 2A-D shows generalizability of gas-MOF delivery. NMR yields were determined by ¹⁹F NMR spectroscopy using fluorobenzene as an internal standard. Isolated yields are shown in parentheses. See supplementary information for experimental details. (A) Scope of Pd-catalyzed Heck coupling of (hetero)aryl bromides and TFP using TFP-Mg₂(dobdc). *XantPhos Pd G3 (2 mol %) and tetrabutylammonium bromide (1 equiv.). (B) Scope of Fe-catalyzed trifluoromethylation of (hetero)arenes using TFMI-Mg₂(dobdc). †0.15 mmol FeSO₄·7H₂O, 50° C., § 50° C. Fc: ferrocene, DMSO: dimethyl sulfoxide (C) Pd-catalyzed defluorinative coupling of HFP and arylboronic acids using HFP-Mg₂(dobdc). (D) User-friendly variations of defluorinative couplings of VDF and arylboronic acids using VDF-Mg₂(dobdc).

FIG. 3 shows structures of Pd G3 precatalysts.

FIG. 4 shows structures of the metal-organic frameworks and the corresponding organic linkers.

FIG. 5 shows PXRD pattern (CuKα radiation, k=1.5418 Å) of MeOH-solvated Mg₂(dobdc) prepared on small scale. The predicted pattern from the single-crystal X-ray diffraction structure of the isostructural framework Zn₂(dobdc) is included for reference.

FIG. 6 shows PXRD pattern (CuKα radiation, k=1.5418 Å) of MeOH-solvated Mg₂(dobdc) prepared on large scale. The predicted pattern from the single-crystal X-ray diffraction structure of the isostructural framework Zn₂(dobdc) is included for reference.

FIG. 7 shows PXRD pattern (CuKα radiation, k=1.5418 Å) of MeOH-solvated Mg₂(m-dobdc). The predicted pattern from the single-crystal X-ray diffraction structure of the isostructural framework Co₂(m-dobdc)•2.0C₂H₄ is included for reference.

FIG. 8 shows PXRD pattern (CuKα radiation, k=1.5418 Å) of MeOH-solvated Ni₂(dobdc). The predicted pattern from the single-crystal X-ray diffraction structure of the isostructural framework Zn₂(dobdc) is included for reference.

FIG. 9 shows PXRD pattern (CuKα radiation, k=1.5418 Å) of MeOH-solvated Ni₂(m-dobdc). The predicted pattern from the single-crystal X-ray diffraction structure of the isostructural framework Co₂(m-dobdc)•2.0C₂H₄ is included for reference.

FIG. 10 shows PXRD pattern (CuKα radiation, k=1.5418 Å) of MeOH-solvated Mn₂(dobdc). The predicted pattern from the single-crystal X-ray diffraction structure of the isostructural framework Zn₂(dobdc) is included for reference.

FIG. 11 shows PXRD pattern (CuKα radiation, k=1.5418 Å) of MeOH-solvated Fe₂(dobdc) packed in a glass capillary. The predicted pattern from the single-crystal X-ray diffraction structure of the isostructural framework Zn₂(dobdc) is included for reference.

FIG. 12 shows PXRD pattern (CuKα radiation, k=1.5418 Å) of MeOH-solvated Co₂(dobdc). The predicted pattern from the single-crystal X-ray diffraction structure of the isostructural framework Zn₂(dobdc) is included for reference.

FIG. 13 shows PXRD pattern (CuKα radiation, k=1.5418 Å) of MeOH-solvated Cu₂(dobdc). The predicted pattern from the single-crystal X-ray diffraction structure of the isostructural framework Zn₂(dobdc) is included for reference.

FIG. 14 shows PXRD pattern (CuKα radiation, k=1.5418 Å) of MeOH-solvated Zn₂(dobdc). The predicted pattern from the single-crystal X-ray diffraction structure of Zn₂(dobdc) is included for reference.

FIG. 15 shows PXRD pattern (CuKα radiation, k=1.5418 Å) of EtOH-solvated Cu₃(btc)₂. The predicted pattern from the single-crystal X-ray diffraction structure of Cu₃(btc)₂ is included for reference.

FIG. 16 shows PXRD pattern (CuKα radiation, k=1.5418 Å) of MeOH-solvated Al₃O(OH)(NH₂-bdc)₃. The predicted pattern from the single-crystal X-ray diffraction structure of the isostructural framework Cr₃(O)(F)(H₂O)₂(bdc)₃•15H₂O is included for reference.

FIG. 17 shows PXRD pattern (CuKα radiation, k=1.5418 Å) of MeOH-solvated Fe₃O(OH)(btc)₂. The predicted pattern from the single-crystal X-ray diffraction structure of Fe₃(O)(F)(H₂O)₂(btc)₂•12.5H₂O is included for reference.

FIGS. 18A-D shows DFT-calculated structures and binding enthalpies of (A) VDF, (B) TFP, (C) TFMI, and (D) HFP in Mg₂(dobdc). Representative carbon, magnesium, fluorine, hydrogen, iodine, and oxygen spheres (representing atoms) are identified.

FIG. 19 shows Rietveld refinement of VDF-Mg₂(dobdc). Measured diffraction data, fitted pattern, and the difference are shown. Weighted residual factor (R_{wp})=11.9%. Inset: Structural model for VDF-Mg₂(dobdc). THF: tetrahydrofuran.

FIGS. 20A-B shows (A) ball-and-stick and (B) space-filling models of VDF-Mg₂(dobdc). Representative carbon, magnesium, fluorine, hydrogen, and oxygen spheres (representing atoms) are identified.

FIG. 21 shows time course of VDF delivery to a solution of THF and Et₂O.

FIG. 22 shows PXRD patterns (CuKα radiation, k=1.5418 Å) of MeOH-solvated Mg₂(dobdc) before and after General Procedures 1-3.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +1-5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent radicals and multivalent radicals, such as, for example, divalent radicals, trivalent radicals, and the like). Illustrative examples of groups include:

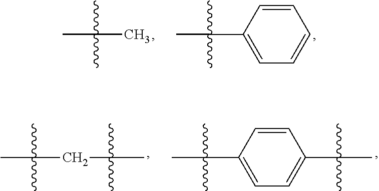

and the like.

As used herein, unless otherwise indicated, a halogenated group or compound comprises (or has) one or more carbon atom(s), each carbon atom comprising (or having) one or more halogen atom(s) (e.g., fluorine, chlorine, bromine, iodine, or the like, or any combination thereof) substituent(s) and, optionally, one or more non-halogen atom substituent(s) (e.g., hydrogen atom substituent(s), or the like, or any combination thereof). As used herein, a perhalogenated group or compound (e.g., a perhalogenated hydrocarbon, also known as a perhalocarbon) is a halogenated group or compound comprising (or having) one or more carbon atom(s), each carbon atom only comprising (or having) one or more halogen atom substituent(s). A halogenated group or compound can comprise (or have) one or more of the same or different halogen atom substituent(s).

As used herein, unless otherwise indicated, a fluorinated group or compound comprises (or has) one or more carbon atom(s), each carbon atom comprising (or having) one or more fluorine atom substituent(s) and, optionally, one or more non-fluorine atom substituent(s) (e.g., hydrogen atom substituent(s), other halogen atom substituent(s), or the like, or any combination thereof). As used herein, a perfluorinated group or compound (e.g., a perfluorinated hydrocarbon, also known as a perfluorocarbon) is a fluorinated group or compound comprising (or having) one or more carbon atom(s), each carbon atom only comprising (or having) one or more fluorine atom substituent(s). Non-limiting examples of fluorinated groups or compounds include vinylidene fluoride (VDF), trifluoromethyl iodide (TFMI), and the like.

As used herein, unless otherwise indicated, an oxocarbon group or compound comprises (or has) one or more carbon-oxygen bond(s) (e.g., carbon-oxygen single bond(s), double bond(s), triple bond(s), or the like, or any combination thereof). In various examples, an oxocarbon group or compound comprises (or has) only oxygen and carbon atoms. In various examples, an oxocarbon group or compound comprises (or has) one or more additional atoms other than oxygen atoms and carbon atoms. Non-limiting examples of oxocarbon groups or compounds include carbon monoxide, carbon dioxide, carbonyl difluoride, trifluoro(trifluoromethoxy)ethene, and the like.

The present disclosure provides, inter alia, methods of storage and delivery of gaseous chemical reactant(s) in a chemical process using metal-organic frameworks (MOFs). The present disclosure also provides methods of making and uses of MOFs and gaseous chemical reactant-loaded (gas-loaded) MOF(s).

In an aspect, the present disclosure provides methods of storage and delivery of one or more gaseous chemical reactant(s) to a chemical process using one or more gas-loaded MOF(s). In various examples, methods use gas-loaded MOF(s) prepared by methods of the present disclosure. Non-limiting examples of methods of storage and delivery of one or more gaseous chemical reactant(s) are disclosed herein.

In various examples, a method of providing one or more gaseous chemical reactant(s) in a chemical process comprises (or consists essentially of or consists of): forming a chemical reaction mixture comprising (or consisting essentially of or consisting of): one or more metal-organic framework(s) (MOF(s)) comprising (or consisting essentially of or consisting of) one or more gaseous chemical reactant(s) (gas-loaded MOF(s)). In various examples, the gaseous chemical reactant(s) is/are material(s) which exist in a gas phase under the reaction conditions of the chemical reaction mixture.

In various examples, each individual MOF comprises (or has or consists essentially of or consists of) the following structure and/or formula: Mₙ(polycarboxylate)ₘ, where M is a metal ion, where n is 1, 2, 3, or the like, and where m is 1, 2, 3, or the like. In various examples, the ratio of n/m may be from about 1:3 to about 3:1, including all ratios of integer n and m values and ranges therebetween (e.g., about 1:1, 1:2, 1:3, 2:1, 3:1, 2:3, or 3:2). In various examples, M is independently at each occurrence chosen from Mg ions, Ni ions, Co ions, Cu ions, Fe ions, Mₙ ions, Cd ions, Zn ions, Al ions, Zr ions, and the like. In various examples, the polycarboxylate is independently at each occurrence chosen from 2,5-dioxido-1,4-benzenedicarboxylate (dobdc⁴⁻), 2,4-dioxidobenzene-1,3-dicarboxylate (m-dobdc⁴⁻), benzene-1,3,5-tricarboxylate (btc³⁻), 1,4-benzenedicarboxylate (bdc²⁻), 2-amino-1,4-benzenedicarboxylate (NH₂-bdc²⁻), and the like. In various examples, each gas-loaded MOF comprises one or more gaseous chemical reactant(s). In various examples, each gas-loaded MOF comprises one or more of the same and/or one or more different gaseous chemical reactant(s) from every other gas-loaded MOF.

It may be desirable to control the release of at least a portion or all of the gaseous chemical reactant(s)) and/or prevent premature release of at least a portion or all of the gaseous chemical reactant(s) in the reaction mixture. In various examples, at least a portion or all of the gas-loaded MOF(s) are present in a delivery vehicle or delivery vehicles, each delivery vehicle configured to control the release of at least a portion or all of the gaseous chemical reactant(s) in the reaction mixture (e.g., release the gaseous chemical reactant(s) at a desired time). In various examples, at least a portion or all of the gas-loaded MOF(s) sequestered in (e.g., encapsulated in or the like) an inert material or the like. Non-limiting examples of inert materials include waxes, polymers, polymeric materials, glasses, and the like, and any combination thereof. In various examples, at least a portion or all of the gas-loaded MOF(s) are present in an inert container or the like (which may be configured to release at least a portion or all of the gas-loaded MOF(s) into the reaction mixture at a desired time). In various examples, gas-loaded MOF(s) can be released from an inert container or the like to solution (e.g., via sonication, vortexing, or the like, of the gas-loaded MOF(s) and/or inert container(s) in the chemical reaction mixture (e.g., solution or the like), dissolving the inert material and/or inert container, melting the inert material and/or inert container, breaking the inert container with scissors, or the like, or any combination thereof). Non-limiting examples of inert containers include wax containers, glass containers (such as, for example, glass capsules, glass ampoules and the like), polymer/polymeric containers (such as, for example, polymer/polymeric glass capsules, polymer/polymeric ampoules and the like) and the like.

In various examples, a chemical process is any chemical process where at least one gaseous chemical reactant is a hazardous gaseous chemical reactant, a sensitive gaseous chemical reactant, an environmentally harmful gaseous chemical reactant (which may be an environmentally destructive gaseous chemical reactant or the like) (e.g., a greenhouse gaseous chemical reactant or the like), or the like, or any combination thereof. In various examples, hazardous gaseous chemical reactant(s) is/are independently at each occurrence chosen from flammable gaseous chemical reactant(s), toxic gaseous chemical reactant(s), radioactive gaseous chemical reactant(s), corrosive gaseous chemical reactant(s), and the like.

In various examples, a chemical process is any chemical process where at least one gaseous chemical reactant is a hazardous gaseous chemical reactant chosen from a flammable gaseous chemical reactant, a toxic gaseous chemical reactant, a radioactive gaseous chemical reactant, a corrosive gaseous chemical reactant, and the like, and any combination thereof. In various examples, sensitive gaseous chemical reactant(s) is/are chosen from oxidatively sensitive gaseous chemical reactant(s), hydrolytically sensitive gaseous chemical reactant(s), and the like.

In various examples, the chemical process is any chemical process where at least one gaseous chemical reactant is a sensitive gaseous chemical reactant chosen from an oxidatively sensitive gaseous chemical reactant, a hydrolytically sensitive gaseous chemical reactant, and the like, and any combination thereof. In various examples, sensitive gaseous chemical reactant(s) is/are chosen from an oxidatively sensitive gaseous chemical reactant, a hydrolytically sensitive gaseous chemical reactant, and the like, and any combination thereof.

In various examples, a chemical process is any chemical process where at least one gaseous chemical reactant is an environmentally harmful gaseous chemical reactant (which may be an environmentally destructive gaseous chemical reactant or the like) chosen from a greenhouse gaseous chemical reactant (e.g., an ozone-depleting gaseous chemical reactant and the like) and the like. In various examples, environmentally harmful gaseous chemical reactant(s) (which may be an environmentally destructive gaseous chemical reactant or the like) is/are independently at each occurrence chosen from greenhouse gaseous chemical reactant(s) (e.g., ozone-depleting gaseous chemical reactant(s) and the like) and the like.

In various examples, the chemical process is any chemical process where at least one of the gaseous chemical reactant(s) is/are a halogenated gaseous chemical reactant, an oxocarbon gaseous chemical reactant, a halogen gaseous chemical reactant, a sulfur gaseous chemical reactant, or the like, or any combination thereof. In various examples, halogen gaseous chemical reactant(s) is/are independently at each occurrence chosen from fluorine, chlorine, xenon difluoride, and the like. In various examples, sulfur gaseous chemical reactant(s) is/are independently at each occurrence chosen from sulfur hexafluoride, sulfuryl fluoride, sulfur dioxide, 1,2-bis(trifluoromethyl)disulfane, trifluoromethyl hypochlorothioite and the like.

In various examples, halogenated gaseous chemical reactant(s) comprise one or more same or different halogen atoms. In various examples, halogenated gaseous chemical reactant(s) is/are hydrocarbon(s) (e.g., alkyl(s), alkene(s), alkyne(s), and the like, and any combination thereof) which is/are partially halogenated or perhalogenated (e.g., partially halogenated or perhalogenated C₁-C₃ hydrocarbon(s) and the like, and any combination thereof) or the like, or any combination thereof.

In various examples, halogenated gaseous chemical reactant(s) comprise only fluorine halogen atoms (e.g., fluorinated gaseous chemical reactant(s)). In various examples, fluorinated gaseous chemical reactant(s) is/are hydrocarbon(s) (e.g., alkyl(s), alkene(s), alkyne(s), and the like) which is/are partially fluorinated or perfluorinated (e.g., partially fluorinated or perfluorinated C₁-C₃ hydrocarbon(s)) or the like, or any combination thereof. In various examples, the fluorinated gaseous chemical reactant(s) is/are independently at each occurrence chosen from vinylidene fluoride (VDF), trifluoropropene (TFP), tetrafluoroethylene (TFE), fluoroethene, fluoroethyne, trifluoropropyne, trifluoroethylene, fluoroform, difluoromethane, hexafluoropropene (HFP), and the like.

In various examples, halogenated gaseous chemical reactant(s) comprise a mixture of types of halogen atoms. In various examples, the halocarbon gaseous chemical reactant(s) is/are hydrocarbon(s) (e.g., alkyl(s), alkene(s), alkyne(s), and the like) which is/are partially halogenated or perhalogenated (e.g., partially halogenated or perhalogenated C₁-C₃ hydrocarbon(s)) comprising a mixture of types of halogen atoms, or the like, or any combination thereof. In various examples, halocarbon gaseous chemical reactant(s) is/are independently at each occurrence chosen from trifluoromethyl iodide (TFMI), trifluoromethyl bromide (TFMBr), trifluoromethyl chloride (TFMCl), trifluoromethylselenyl chloride, trifluoromethanethiol, difluoroiodomethane, difluorobromomethane, difluorochloromethane, fluoroiodomethane, fluorobromomethane, fluorochloromethane, and the like.

In various examples, oxocarbon gaseous chemical reactant(s) comprise one or more carbon-oxygen bonds. In various examples the oxocarbon gaseous chemical reactant(s) comprise only oxygen and carbon atoms. In various examples, oxocarbon gaseous chemical reactant(s) comprise one or more additional atoms other than oxygen atoms and carbon atoms. In various examples, the oxocarbon gaseous chemical reactant(s) is/are independently at each occurrence chosen from carbon monoxide, carbonyl difluoride, trifluoro(trifluoromethoxy)ethene, and the like.

Gas-loaded MOF(s) can comprise various amounts of gaseous chemical reactant(s). In various examples, gas-loaded MOF(s), at about −196° C. to about 55° C. (e.g., about 30° C.), on average, comprise about 0.1 millimole (mmol) gaseous chemical reactant(s)/gram gas-loaded MOF(s) to about 10 mmol gaseous chemical reactant(s)/gram gas-loaded MOF(s), including all 0.01 mmol gaseous chemical reactant(s)/gram gas-loaded MOF(s) values and ranges therebetween.

In various examples, gas-loaded MOF(s) release(s) at least a portion of, substantially all of, or all of the one or more gaseous chemical reactant(s) in from about less than 3 seconds to about less than 24 hours, including all integer second values and ranges therebetween (e.g., from about less than 30 seconds to about less than 20 hours, from about less than 5 minute to about less than 15 hours, from about less than 10 minutes to about less than 10 hours, from about less than 15 minutes to about less than 5 hours, from about less than 20 minutes to about less than 3 hours, or from about less than 30 minutes to about less than 1 hour, including all integer second values and ranges therebetween).

Gas-loaded MOF(s) can release chemical reactant(s) at various temperatures. In various examples, gas-loaded MOF(s) release(s) at least a portion of, substantially all of, or all of the one or more gaseous chemical reactant(s) a temperature or temperatures of about −80° C. to about 180° C., including all 0.1° C. values and ranges therebetween. In various examples, the gas can be released from the gas-loaded MOF at room temperature (e.g., from about 20° C. to about 22° C., including all 0.1° C. values and ranges therebetween), below room temperature (e.g., at about 19° C. or below, such as for example, from about −80° C. to about 19° C., including all 0.1° C. values and ranges therebetween) (e.g., about −10° C., about −50° C., about −80° C.), above room temperature (e.g., at about 23° C. or above, e.g. from about 23° C. to about 180° C., including all 0.1° C. values and ranges therebetween) (e.g., about 100° C., about 150° C., about 200° C.), or any combination thereof.

Various chemical reactions can be performed. In various examples, the chemical process is chosen from Negishi coupling, Heck coupling, trifluoromethylation, defluorinative cross-coupling, carbonylative Suzuki coupling, difluoromethylation, copper-catalyzed borylation, hydroarylation, aminocarbonylation, olefin metathesis, fluoroalkylation, deoxyfluorination, deoxyfluoroalkoxylation, fluoroalkylthiolation, fluoroalkylselenation, fluorovinylation, fluoroalkynylation, pentafluorosulfanylation, and the like.

In various examples, the chemical reaction mixture further comprises one or more additional chemical reactant(s). Various additional chemical reactant(s) can be used. In various examples, the additional chemical reactant(s) is/are those necessary to carry out one or more of the various chemical reaction(s) described herein. In various examples, the additional chemical reactant(s) is/are additional gaseous chemical reactant(s) (e.g., of the present disclosure) optionally comprised in one or more additional gas-loaded MOF(s) (e.g., of the present disclosure and/or prepared by a method of the present disclosure). In various examples, the chemical reaction mixture is maintained under inert and/or anhydrous conditions. In various examples, a chemical reaction of the chemical process occurs between at least a portion of, substantially all of, or all of the gaseous chemical reactant(s) and at least a portion of, substantially all of, or all of the additional chemical reactant(s) in the chemical reaction mixture. In various examples, prior to the chemical reaction, the gas-loaded MOF(s) release(s) at least a portion of, substantially all of, or all of the gaseous chemical reactant(s) into the chemical reaction mixture.

In various examples, the gaseous chemical reactant(s) is/are material(s) which exist in a gas phase under the reaction conditions (e.g., temperature, pressure, and the like, and any combination thereof) of the chemical reaction mixture (e.g., gas(es), volatile liquid(s), sublimatable solid(s), and the like, and any combination thereof). In various examples, the gaseous chemical reactant(s) is/are independently at each occurrence chosen from hazardous gaseous chemical reactant(s), sensitive gaseous chemical reactant(s), environmentally harmful gaseous chemical reactant(s), and the like, and any combination thereof. In various examples, each gas-loaded MOF comprises one or more gaseous chemical reactant(s). In various examples, one or more or all of the gaseous chemical reactant(s) of two or more or all of the gas-loaded MOF(s) are the same. In various examples, one or more or all of the gaseous chemical reactant(s) of two or more or all of the gas-loaded MOF(s) are different.

In various examples, a chemical reaction mixture further comprises one or more solvent(s). Various solvents can be used. In various examples, the solvent(s) is/are chosen from organic solvents, ionic liquids, water, and the like, and any combination thereof. In various examples, the organic solvent(s) is/are chosen from polar aprotic solvent(s) (e.g., anisole, N,N-dimethylformamide, dimethyl sulfoxide, acetonitrile, or the like, or any combination thereof), ether solvent(s) (e.g., tetrahydrofuran, diethyl ether, dioxane, or the like, or any combination thereof), or the like, or any combination thereof. In various examples, the solvent(s) is/are anhydrous and/or oxygen-free solvent(s) or the like. In various examples, the organic solvent(s) is/are chosen from nonpolar solvent(s) (e.g., toluene, hexanes, pentane, chlorinated solvents (e.g., dichloromethane, chloroform, or the like, or any combination thereof), or the like, or any combination thereof), or the like, or any combination thereof.

A reaction can be performed under various reaction conditions. A reaction can comprise one or more steps and each step can be performed under the same or different reaction conditions as other steps. A reaction can be carried out at various temperatures. In various examples, a reaction is carried out at room temperature (e.g., from about 20° C. to about 22° C., including all 0.1° C. values and ranges therebetween), below room temperature (e.g., at about 0° C. or below, such as for example, from about −200° C. to about 0° C., including all 0.1° C. values and ranges therebetween) (e.g., about −10° C., about −50° C., about −100° C., about −150° C., or about −200° C.), above room temperature (e.g., at a temperature up to or about a boiling point of the solvent(s), if present) (e.g., at about 100° C. or above, e.g. from about 100° C. to about 400° C., including all 0.1° C. values and ranges therebetween) (e.g., about 100° C., about 200° C., about 300° C., about 400° C., or about 500° C.), or any combination thereof (e.g., where each step is performed at a different temperature as other steps).

A reaction can be carried out at various pressures. In various examples, a reaction is carried out at atmospheric pressure (e.g., 1 standard atmosphere (atm) at sea level), at greater than atmospheric pressure (e.g. heating in a sealed pressurized reaction vessel and the like), at below atmospheric pressure (e.g., under vacuum (e.g., from about 1 mTorr or less to about 100 mTorr or less, including all 0.1 mTorr values and ranges therebetween) (e.g., about 100 mTorr or less, about 50 mTorr or less, about 10 mTorr or less, or about 1 mTorr or less) and the like), or any combination thereof (e.g., where each step is performed at a different pressure as other steps).

A reaction can be carried out for various times. The reaction time can depend on factors such as, for example, temperature, pressure, presence and/or efficiency of a catalyst, presence and/or intensity of an applied energy source, stirring, grinding, or the like, or a combination thereof. In various examples, reaction times range from about seconds (e.g., two seconds) to greater than about 200 hours, including all integer second values and ranges therebetween (e.g., from about 1 minute to about 150 hours, including all integer second values and ranges therebetween) (e.g., about 10 minutes, about 1 hour, about 12 hours, about 24 hours, about 120 hours, or about 150 hours), or any combination thereof (e.g., where each step is performed at a different time as other steps).

In an aspect, the present disclosure provides methods of making gaseous chemical reactant-loaded (gas-loaded) MOF(s) for the storage and delivery of the gaseous chemical reactant(s) in a chemical process. Methods of making the gas-loaded MOF(s) are based on contacting the MOF(s) with one or more gaseous chemical reactant(s). Non-limiting examples of methods of making gas-loaded MOF(s) are disclosed herein.

In various examples, a method of making one or more gas-loaded MOF(s) comprises (or consists essentially of or consists of): forming a MOF gas-loading reaction mixture comprising (or consisting essentially of or consisting of): the gaseous chemical reactant(s); and the MOF(s), where the gas-loaded MOF(s) is/are formed. In various examples, a method further comprises: activating the MOF(s) prior to forming the MOF gas-loading reaction mixture; cooling the gas-loaded MOF(s); maintaining the gas-loaded MOF(s) under inert and/or anhydrous conditions; isolating and/or activating the gas-loaded MOF(s); using the gas-loaded MOF(s) in a chemical reaction mixture of the present disclosure; or any combination thereof.

A MOF can comprise various amounts of gaseous chemical reactant(s). In various examples, a MOF gas-loading reaction mixture comprises about 0.1 equivalent(s) (eq) to about 100 eq of gaseous chemical reactant(s) (independently or in the aggregate), including all 0.1 eq values and ranges therebetween (e.g., about 1 eq to about 10 eq or about 1 eq to about 50 eq), based on the total equivalents of metal ions (e.g., Mg, Ni, Co, Cu, Fe, Mₙ, Cd, Zn, Al, Zr, and the like) of the MOF(s) in the MOF gas-loading reaction mixture. In various examples, the gaseous chemical reactant(s) is/are present in the MOF gas-loading reaction mixture in a gas phase, a liquid phase, or as a solution (e.g., in an organic solvent, such as, for example, tetrahydrofuran, hexanes, toluene, or the like, or any combination thereof).

In various examples, a MOF gas-loading reaction mixture is formed with or without mixing (e.g., stirring or the like). In various examples, MOF(s) are contacted (e.g., dosed) with gaseous chemical reactant(s) incrementally.

In various examples, a MOF gas-loading reaction mixture is heated after forming the gas-loaded MOF(s) and/or after activation of the gas-loaded MOF(s). In various examples, cooling the gas-loaded MOF(s) comprises subjecting the gas-loaded MOF(s) to a temperature of from about −200 degrees Celsius (° C.) to about 80° C., including all 0.1° C. values and ranges therebetween, for about 1 minute to about 24 hours or longer, including all integer minute values and ranges therebetween.

In various examples, the gas-loaded MOF(s) are cooled after forming the gas-loaded MOF(s). In various examples, cooling the gas-loaded MOF(s) comprises subjecting the gas-loaded MOF(s) to a temperature of from about −200 degrees Celsius (° C.) to about 50° C., including all 0.1° C. values and ranges therebetween, for about 1 minute to about 60 minutes, including all integer second values and ranges therebetween.

In various examples, isolating the gas-loaded MOF(s) comprises separating the gas-loaded MOF(s) from MOF gas-loading reaction mixture (e.g., a gas phase mixture, a liquid phase mixture, or a solution-phase mixture mixture) or the like. In various examples, isolating the gas-loaded MOF(s) comprises filtering or centrifuging the MOF gas-loading reaction mixture, or the like, or any combination thereof. In various examples, isolating the gas-loaded MOF(s) further comprises purifying the separated gas-loaded MOF(s) or the like. In various examples, purifying the separated gas-loaded MOF(s) comprises rinsing the separated gas-loaded MOF(s) with solvent, solvent-exchanging the separated gas-loaded MOF(s), desolvating the separated gas-loaded MOF(s), or the like, or any combination thereof. In various examples, rinsing the separated gas-loaded MOF(s) with solvent and/or solvent-exchanging the separated gas-loaded MOF(s) is/are performed with one or more solvent(s) chosen from organic solvents (such as, for example, N,N-dimethylformamide, and the like, and any combination thereof), alcohols (such as, for example methanol, ethanol, and the like, and any combination thereof), and the like, or the like.

In various examples, activating the MOF(s) (e.g., isolated MOF(s) and the like) and/or the gas-loaded MOF(s) (e.g., isolated gas-loaded MOF(s) and the like) comprises heating the MOF(s) (e.g., isolated MOF(s) and the like) and/or the gas-loaded MOF(s) (e.g., isolated gas-loaded MOF(s) and the like) or the like, optionally, under vacuum and/or an inert atmosphere, or the like, or any combination thereof. In various examples, activating the MOF(s) (e.g., isolated MOF(s) and the like) and/or the gas-loaded MOF(s) (e.g., isolated gas-loaded MOF(s) and the like) comprises heating the MOF(s) (e.g., isolated MOF(s) and the like) and/or the gas-loaded MOF(s) (e.g., isolated gas-loaded MOF(s) and the like) at a temperature of from about 30 degrees Celsius (° C.) to about 300° C., including all 0.1° C. values and ranges therebetween, or the like. In various examples, activating the MOF(s) (e.g., isolated MOF(s) and the like) and/or the gas-loaded MOF(s) (e.g., isolated gas-loaded MOF(s) and the like) comprises heating the MOF(s) (e.g., isolated MOF(s) and the like) and/or the gas-loaded MOF(s) (e.g., isolated gas-loaded MOF(s) and the like) at a pressure of from about 10 microbar(s) (pbar) to about 1000 millibar(s) (mbar), including all 0.1 pbar values and ranges therebetween or the like. In various examples, activating the MOF(s) (e.g., isolated MOF(s) and the like) and/or the gas-loaded MOF(s) (e.g., isolated gas-loaded MOF(s) and the like) comprises heating the MOF(s) (e.g., isolated MOF(s) and the like) and/or the gas-loaded MOF(s) (e.g., isolated gas-loaded MOF(s) and the like) under a flow of nitrogen or the like.

Gas-loaded MOF(s) can be stored before use. In various examples, gas-loaded MOF(s) is/are maintained under inert (e.g., oxygen-free, inert gas, or the like, or any combination thereof) and/or anhydrous conditions or the like and/or at low temperature(s) (such as, for example, from about −196° C. to about 0° C., including all 0.1° C. values and ranges therebetween). In various examples, gas-loaded MOF(s) is/are stored for extended periods of time (from about 1 day to 1 about week or more, including all 0.1 s (second) values and ranges therebetween).

In an aspect, the present disclosure provides methods of recycling gas-loaded MOF(s) from which a portion of, substantially all of, or all of the gaseous chemical reactant(s) has/have been released into the chemical reaction mixture and/or reacted with at least a portion of, substantially all of, or all of the additional chemical reactant(s) in the chemical reaction mixture (e.g. spent gas-loaded MOF(s)). Non-limiting examples of methods of recycling spent gas-loaded MOF(s) are disclosed herein.

In various examples, a method of recycling MOF(s) comprises isolating and/or activating the spent gas-loaded MOF(s), where recycled MOF(s) is/are formed. In various examples, one or more of the recycled MOF(s) is/are used to form one or more gas-loaded MOF(s) (e.g., of the present disclosure).

In various examples, isolating the spent gas-loaded MOF(s) comprises separating the spent gas-loaded MOF(s) from the chemical reaction mixture or the like. In various examples, isolating the spent gas-loaded MOF(s) comprises filtering or centrifuging the chemical reaction mixture, or the like, or any combination thereof. In various examples, isolating the spent gas-loaded MOF(s) further comprises purifying the separated spent gas-loaded MOF(s) or the like. In various examples, purifying the separated spent gas-loaded MOF(s) comprises rinsing the separated spent gas-loaded MOF(s) with solvent, solvent-exchanging the separated spent gas-loaded MOF(s), desolvating the separated spent gas-loaded MOF(s), or the like, or any combination thereof. In various examples, rinsing the separated spent gas-loaded MOF(s) with solvent and/or solvent-exchanging the separated spent gas-loaded MOF(s) is/are performed with one or more solvent(s) chosen from organic solvents (such as, for example, N,N-dimethylformamide, and the like, and any combination thereof), alcohols (such as, for example methanol, ethanol, and the like, and any combination thereof), and the like, or the like.

In various examples, activating the spent gas-loaded MOF(s) (e.g., isolated spent gas-loaded MOF(s) and the like) comprises heating the spent gas-loaded MOF(s) (e.g., isolated spent gas-loaded MOF(s) and the like), optionally, under vacuum, optionally, under an inert atmosphere, or the like. In various examples, activating the spent gas-loaded MOF(s) (e.g., isolated spent gas-loaded MOF(s) and the like) comprises heating the spent gas-loaded MOF(s) (e.g., isolated spent gas-loaded MOF(s) and the like) at a temperature of from about 30 degrees Celsius (° C.) to about 300° C., including all 0.1° C. values and ranges therebetween. In various examples, activating the spent gas-loaded MOF(s) (e.g., isolated spent gas-loaded MOF(s) and the like) comprises heating the spent gas-loaded MOF(s) (e.g., isolated spent gas-loaded MOF(s) and the like) at a pressure of from about 10 microbar(s) (pbar) to about 1000 millibar(s) (mbar), including all 0.1 pbar values and ranges therebetween. In various examples, activating the spent gas-loaded MOF(s) (e.g., isolated spent gas-loaded MOF(s) and the like) comprises heating the spent gas-loaded MOF(s) (e.g., isolated spent gas-loaded MOF(s) and the like) under the flow of nitrogen or the like.

In an aspect, the present disclosure provides methods of making MOF(s) (e.g., the MOF(s) used to make the gas-loaded MOF(s) of the present disclosure). Non-limiting examples of methods of making MOF(s) are disclosed herein.

In various examples, a method makes MOF(s), each individual MOF comprising (or having or consisting essentially of or consisting of) the following formula and/or structure: Mₙ(polycarboxylate)ₘ, where M is a metal ion independently at each occurrence chosen from Mg ions, Mₙ ions, Co ions, Ni ions, Cu ions, Zn ions, Fe ions, Al ions, and the like, where n is 1, 2, 3, or the like, and where m is 1, 2, 3 or the like.

In various examples, a method of making MOF(s) comprises (or consists essentially of or consists of): forming a first MOF reaction mixture comprising (or consisting essentially of or consisting of): one or more compound(s) independently comprising (or consisting essentially of or consisting of) one or more of the metal ion(s) (e.g., the compound(s) may be salt(s) independently comprising the metal ion(s) (such as, for example, magnesium salts (e.g., Mg(NO₃)₂, Mg(NO₃)₂•6H₂O, or the like), nickel salts (e.g., NiCl₂, Ni(NO₃)₂, Ni(NO₃)₂•6H₂O, or the like), manganese salts (e.g., MnCl₂, MnCl₂•4H₂O, and the like), iron salts (e.g., FeCl₂, Fe(NO₃)₃, Fe(NO₃)₃•9H₂O, and the like), cobalt salts (e.g., Co(NO₃)₂, Co(NO₃)₂•6H₂O, and the like), copper salts (Cu(NO₃)₂, Cu(NO₃)₂•2.5H₂O, Cu(Oac)₂, Cu(Oac)₂•H₂O, and the like), zinc salts (e.g., Zn(NO₃)₂, Zn(NO₃)₂•6H₂O, and the like), aluminum salts (e.g., AlCl₃•6H₂O and the like), and the like, and hydrates thereof, and any combination thereof), or the like, or any combination thereof; one or more polycarboxylic acid(s) (e.g., 2,5-dihydroxyterephthalic acid (H₄dobdc), 1,3,5-benzenetricarboxylic acid (H₃btc), 4,6-dihydroxyisophthalic acid (m-H₄dobdc), 2-aminoterephthalic acid (NH₂—H₂bdc) or the like) (e.g., polycarboxylic acid(s) comprising a terminal amino group (which may form an amide group, or the like), salt(s) thereof (e.g. Na salts, K salts, and the like, and any combination thereof), or the like, or any combination thereof; and optionally, one or more basic solvent(s) (e.g., polar aprotic solvent(s) (such as, for example, N,N-dimethylformamide (DMF) and the like), one or more non-basic solvents(s) (such as, for example, water at pH about 7.0, alcohol(s) (e.g., ethanol, methanol, and the like, and any combination thereof), or the like, or any combination thereof) or the like, or any combination thereof. In various examples, the reaction mixture comprises (or consists essentially of or consists of) 0.1 mol/liter (M) or greater (e.g., 0.5M or greater, 1M or greater, 1.5M or greater, 2M or greater, 5M or greater, or 10M or greater)(e.g., from about 0.1 M or greater to about 10M or greater, including all 0.05 M values and ranges therebetween) of: the compound(s) comprising the metal ion(s), or the like, or any combination thereof; the polycarboxylic acid(s) and/or the salt(s) thereof, or the like, or any combination thereof; or both. In various examples, a reaction mixture is held (or heated to depending on the temperature of the reaction mixture after formation) at a temperature of about −150 degrees Celsius (° C.) to about 200° C., including all 0.1° C. values and ranges therebetween (e.g., about 30 degrees Celsius (° C.) to about 150° C.). In various examples, a method further comprises heating the reaction mixture (such as, for example, heating the reaction mixture to a temperature of from about 30 degrees Celsius (° C.) to about 200° C.). In various examples, one or more first solid(s) is/are formed. In various examples, a method comprises isolating the first solid(s).

In various examples, a method further comprises: forming a second MOF reaction mixture comprising: the first solid(s); and one or more basic solvent(s) (e.g., polar aprotic solvent(s) such as, for example, N,N-dimethylformamide (DMF), and the like), one or more non-basic solvents(s) (e.g., water at pH about 7.0, alcohol(s) (e.g., ethanol, methanol, or the like, or any combination thereof), or the like, or any combination thereof) or the like, or any combination thereof, where the one or more MOF(s) is/are formed. In various examples, a method comprises maintaining the second MOF reaction mixture under inert and/or anhydrous conditions. In various examples, a method further comprises isolating and/or activating the MOF(s) (e.g., as disclosed herein).

In various examples, the first and/or the second MOF reaction mixture(s) is/are maintained with or without stirring. In various examples, the first MOF reaction mixture is heated with or without refluxing the solvent. In various examples, the first MOF reaction mixture is heated at about the boiling temperature of the solvent(s) (e.g., in a high-pressure reaction vessel, under reflux, or the like).

In various examples, isolating the first solid(s) comprises separating the first solid(s) from the first MOF reaction mixture or the like. In various examples, isolating the MOF(s) comprises separating the MOF(s) from the second MOF reaction mixture or the like. In various examples, isolating the first solid(s) and/or MOF(s) comprises filtering and/or centrifuging, or the like, the first and/or second MOF reaction mixture, respectively. In various examples, isolating the first solid(s) and/or the MOF(s) further comprises purifying the separated first solid(s) and/or MOF(s) or the like. In various examples, purifying the separated first solid(s) and/or MOF(s) comprises rinsing the separated first solid(s) and/or MOF(s) with solvent, solvent-exchanging the separated first solid(s) and/or MOF(s), desolvating the separated first solid(s) and/or MOF(s), or the like, or any combination thereof. In various examples, rinsing the separated first solid(s) and/or MOF(s) with solvent and/or solvent-exchanging the separated first solid(s) and/or MOF(s) is/are performed with one or more solvent(s) chosen from organic solvents (such as, for example, N,N-dimethylformamide, and the like, and any combination thereof), alcohols (such as, for example methanol, ethanol, and the like, and any combination thereof), and the like, or the like.

In various examples, activating the MOF(s) (e.g., isolated MOF(s) and the like) comprises heating the MOF(s) (e.g., isolated MOF(s) and the like), optionally, under vacuum and/or an inert atmosphere, or the like, or any combination thereof. In various examples, activating the MOF(s) (e.g., isolated MOF(s) and the like) comprises heating the MOF(s) (e.g., isolated MOF(s) and the like) at a temperature of from about 30 degrees Celsius (° C.) to about 300° C., including all 0.1° C. values and ranges therebetween. In various examples, activating the MOF(s) (e.g., isolated MOF(s) and the like) comprises heating the MOF(s) (e.g., isolated MOF(s) and the like) at a pressure of from about 10 microbar(s) (pbar) to about 1000 millibar(s) (mbar), including all 0.1 pbar values and ranges therebetween. In various examples, activating the MOF(s) (e.g., isolated MOF(s) and the like) comprises heating the MOF(s) (e.g., isolated MOF(s) and the like) under a flow of nitrogen or the like.

The following Statements describe various examples of MOFs, compositions, and methods of the present disclosure and are not intended to be in any way limiting:
Statement 1. A method of providing one or more gaseous chemical reactant(s) in a chemical process, the method comprising: forming a chemical reaction mixture comprising: one or more metal-organic framework(s) (MOF(s)) comprising one or more gaseous chemical reactant(s) (gas-loaded MOF(s)), where each individual MOF comprises (or has) the following structure and/or formula: Mₙ(polycarboxylate)ₘ, where M is a metal ion, where n is 1, 2, 3, or the like, and where m is 1, 2, 3, or the like, and where the gaseous chemical reactant(s) is/are material(s) which exist in a gas phase under the reaction conditions (e.g., temperature, pressure, and the like, and any combination thereof) of the chemical reaction mixture (e.g., gas(es), volatile liquid(s), sublimatable solid(s), and the like, and any combination thereof); optionally, one or more additional chemical reactant(s); optionally, where the chemical reaction mixture is maintained under inert and/or anhydrous conditions, and where a chemical reaction of the chemical process occurs between at least a portion of, substantially all of, or all of the gaseous chemical reactant(s) and at least a portion of, substantially all of, or all of the additional chemical reactant(s), if present.
Statement 2. A method according to Statement 1, where M is independently at each occurrence chosen from Mg ions, Ni ions, Co ions, Cu ions, Fe ions, Mₙ ions, Cd ions, Zn ions, Al ions, Zr ions, and the like.
Statement 3. A method according to Statement 1 or Statement 2, where the polycarboxylate is independently at each occurrence chosen from 2,5-dioxido-1,4-benzenedicarboxylate (dobdc⁴⁻), 2,4-dioxidobenzene-1,3-dicarboxylate (m-dobdc⁴⁻), benzene-1,3,5-tricarboxylate (btc³⁻), 1,4-benzenedicarboxylate (bdc²⁻), 2-amino-1,4-benzenedicarboxylate (NH₂-bdc²⁻), and the like.
Statement 4. A method according to any one of Statements 1-3, where the ratio of n/m is from about 1:3 to about 3:1, including all 0.1 values and ranges therebetween (e.g., about 1:1, 1:2, 1:3, 2:1, 3:1, 2:3, or 3:2).
Statement 5. A method according to any one of Statements 1-4, where each individual MOF comprises (or has) the following structure and/or formula: Mg₂(dobdc), Mn₂(dobdc), Fe₂(dobdc), Co₂(dobdc), Ni₂(dobdc), Cu₂(dobdc), Zn₂(dobdc), Cd₂(dobdc) Mg₂(m-dobdc), Ni₂(m-dobdc), Cu₃(btc)₂, Fe₃O(OH)(btc)₂, Al₃O(OH)(NH₂-bdc)₃, Zeolite Y, and the like.
Statement 6. A method according to any one of Statements 1-5, where the gaseous chemical reactant(s) is/are independently at each occurrence chosen from hazardous gaseous chemical reactant(s), sensitive gaseous chemical reactant(s), environmentally harmful gaseous chemical reactant(s), and the like, and any combination thereof.
Statement 7. A method according to any one of Statements 1-6, where the gaseous chemical reactant(s) is/are chosen from halogenated gaseous chemical reactant(s) oxocarbon gaseous chemical reactant(s), halogen gaseous chemical reactant(s), sulfur gaseous chemical reactant(s), and the like, and any combination thereof.
Statement 8. A method according to any one of Statements 1-7, where the additional chemical reactant(s) is/are chosen from Negishi coupling reactant(s), Heck coupling reactant(s), trifluoromethylation reactant(s), defluorinative cross-coupling reactant(s), carbonylative Suzuki coupling reactant(s), copper-catalyzed borylation reactant(s), hydroarylation reactant(s), aminocarbonylation reactant(s), olefin metathesis reactant(s), fluoroalkylation reactant(s), deoxyfluorination reactant(s), deoxyfluoroalkoxylation reactant(s), fluoroalkylthiolation reactant(s), fluoroalkylselenation reactant(s), fluorovinylation reactant(s), fluoroalkynylation reactant(s), pentafluorosulfanylation reactant(s), and the like.
Statement 9. A method according to any one of Statements 1-8, where the chemical reaction mixture comprises one or more solvent(s).
Statement 10. A method according to Statement 9, where the solvent(s) is/are chosen from organic solvents, ionic liquids, water, and the like, and any combination thereof.
Statement 11. A method according to any one of Statements 1-10, where the gas-loaded MOF(s), at about −196° C. to about 55° C. (e.g., about 30° C.), on average, from about 0.1 millimole (mmol) gaseous chemical reactant(s)/gram gas-loaded MOF(s) to about 10 mmol gaseous chemical reactant(s)/gram gas-loaded MOF(s), including all 0.01 mmol gaseous chemical reactant(s)/gram gas-loaded MOF(s) values and ranges therebetween.
Statement 12. A method according to any one of Statements 1-11, the method further comprising, prior to the occurrence of the chemical reaction, releasing at least a portion of, substantially all of, or all of the gaseous chemical reactant(s) from the gas-loaded MOF(s) into the chemical reaction mixture.
Statement 13. A method according to Statement 12, where the releasing at least a portion of, substantially all of, or all of the gaseous chemical reactant(s) from the gas-loaded MOF(s) into the chemical reaction mixture is achieved by increasing the chemical reaction mixture temperature, reducing the chemical reaction mixture pressure, adding a coordinating solvent to displace the gas, irradiation of the reaction mixture with light, sonication of the reaction mixture, mechanical grinding of the reaction mixture, or the like, or any combination thereof.
Statement 14. A method according to any one of Statements 1-13, the method further comprising, prior to forming the chemical reaction mixture: forming the gas-loaded MOF(s); optionally, cooling the gas-loaded MOF(s); and optionally, maintaining the gas-loaded MOF(s) under inert and/or anhydrous conditions and/or at low temperatures (from about −196° C. to about 0° C., including all 0.1° C. values and ranges therebetween) (e.g., for extended periods of time (from about 1 day to 1 about week or more, including all 0.1 s (second) values and ranges therebetween).
Statement 15. A method according to Statement 14, where the forming the gas-loaded MOF(s) comprises: optionally, activating the MOF(s) (e.g., as disclosed herein); forming a MOF gas-loading reaction mixture comprising: the gaseous chemical reactant(s); and the MOF(s), where the gas-loaded MOF(s) is/are formed, and optionally, isolating and/or activating the gas-loaded MOF(s) (e.g., as disclosed herein).
Statement 16. A method according to Statements 14 or Statement 15, the method further comprising, prior to forming the gas-loaded MOF(s), forming the MOF(s) (e.g., according to a method of the present disclosure, such as for example Statements 18-20).
Statement 17. A method according to any one of Statements 1-16, the method further comprising, after the gas-loaded MOF(s) has/have released a portion of, substantially all of, or all of the gaseous chemical reactant(s) into the chemical reaction mixture and/or has/have reacted with at least a portion of, substantially all of, or all of the additional chemical reactant(s) in the chemical reaction mixture, where spent gas-loaded MOF(s) are formed, isolating and/or activating the spent gas-loaded MOF(s), where recycled MOF(s) is/are formed.
Statement 18. A method of making one or more metal-organic framework(s) (MOF(s)), where each individual MOF comprises (or has) the following formula and/or structure: Mₙ(polycarboxylate)ₘ, where M is a metal ion independently at each occurrence chosen from Mg ions, Mₙ ions, Co ions, Ni ions, Cu ions, Zn ions, Fe ions, and Al ions, and the like, where and the like, where n is 1, 2, 3, or the like, and where m is 1, 2, 3 or the like, the method comprising: forming a first MOF reaction mixture comprising: one or more compound(s) comprising one or more of the metal ion(s) (e.g., the compound(s) may be salt(s) comprising the metal ion(s) such as, for example, Mg(NO₃)₂, Mg(NO₃)₂•6H₂O, Ni(NO₃)₂•6H₂O, MnCl₂•4H₂O, NiCl₂, FeCl₂, Co(NO₃)₂•6H₂O, Cu(NO₃)₂•2.5H₂O, Zn(NO₃)₂•6H₂O, Cu(Oac)₂•H₂O, AlCl₃•6H₂O, Fe(NO₃)₃•9H₂O, and hydrates thereof, and the like, and any combination thereof) or the like, or any combination thereof; one or more polycarboxylic acid(s) (e.g., 2,5-dihydroxyterephthalic acid (H₄dobdc), 1,3,5-benzenetricarboxylic acid (H₃btc), 4,6-dihydroxyisophthalic acid (m-H₄dobdc), 2-aminoterephthalic acid (NH₂—H₂bdc) or the like), salt(s) thereof (e.g., Na salts, K salts, and the like, and any combination thereof), or the like, or any combination thereof; and one or more basic solvent(s) (e.g., polar aprotic solvent(s) such as, for example, N,N-dimethylformamide (DMF) and the like), one or more non-basic solvents(s) (e.g., water at pH about 7.0, alcohol(s) (e.g., ethanol, methanol, and the like, and any combination thereof), or the like, or any combination thereof) or the like, or any combination thereof, where the reaction mixture comprises 0.1 mol/liter (M) or greater (e.g., 0.5M or greater, 1M or greater, 1.5M or greater, 2M or greater, 5M or greater, or 10M or greater)(e.g., from about 0.1 M or greater to about 10M or greater, including all 0.05M values and ranges therebetween) of: the compound(s) comprising the metal ion(s), or the like, or any combination thereof; the polycarboxylic acid(s) and/or the salt(s) thereof, or the like, or any combination thereof; or both; and heating the reaction mixture, where one or more first solid(s) is/are formed; optionally, isolating the first solid(s); forming a second MOF reaction mixture comprising: the first solid(s); and one or more basic solvent(s) (e.g., polar aprotic solvent(s) such as, for example, N,N-dimethylformamide (DMF), and the like), one or more non-basic solvents(s) (e.g., water at pH about 7.0, alcohol(s) (e.g., ethanol, methanol, or the like, or any combination thereof), or the like, or any combination thereof) or the like, or any combination thereof, optionally, where the second MOF reaction mixture is maintained under inert and/or anhydrous conditions, where the one or more MOF(s) is/are formed; and optionally, isolating and/or activating the MOF(s) (e.g., as disclosed herein).
Statement 19. A method according to Statement 18, where the metal compound(s) comprise Mg(NO₃)₂, Mg(NO₃)₂•6H₂O, NiCl₂, Ni(NO₃)₂, Ni(NO₃)₂•6H₂O, MnCl₂, MnCl₂•4H₂O, FeCl₂, Fe(NO₃)₃, Fe(NO₃)₃•9H₂O, Co(NO₃)₂, Co(NO₃)₂•6H₂O, Cu(NO₃)₂, Cu(NO₃)₂•2.5H₂O, Cu(Oac)₂, Cu(Oac)₂•H₂O, Zn(NO₃)₂, Zn(NO₃)₂•6H₂O, AlCl₃•6H₂O, and the like, and hydrates thereof, or the like, or any combination thereof.
Statement 20. A method according to Statement 18 or Statement 19, where the polycarboxylic acid(s) is/are independently at each occurrence chosen from 2,5-dihydroxyterephthalic acid (H₄dobdc), 1,3,5-benzenetricarboxylic acid (H₃btc), 4,6-dihydroxyisophthalic acid (m-H₄dobdc), 2-aminoterephthalic acid (NH₂—H₂bdc), and partially or completely deprotonated structural analogs thereof, and the like.
Statement 21. A method according to any one of Statements 18-20, where the first MOF reaction mixture comprise from about 1 equivalent(s) (eq) to about 10 eq (independently or in the aggregate) of polycarboxylic acid(s), polycarboxylate salt(s) (e.g. Na salts, K salts, or the like, or any combination thereof), or the like, or any combination thereof, including all 0.1 eq values and ranges therebetween, based on the total equivalents of metal (M).
Statement 22. A method according to any one of Statements 18-21, where the first MOF reaction mixture is heated at a temperature of from about 30 degrees Celsius (° C.) to about 150° C., including all 0.1° C. values and ranges therebetween.

The steps of the methods described in the various embodiments and examples disclosed herein are sufficient to carry out the methods of the present disclosure. Thus, in various examples, a method consists essentially of a combination of the steps of the methods disclosed herein. In various other examples, a method consists of such steps.

The following examples are presented to illustrate the present disclosure. It is not intended to be limiting in any matter.

### Example 1

The following is an example of methods of storage and delivery of gaseous chemical reactant(s) in a chemical process using metal-organic frameworks (MOFs), methods of making and uses of MOFs and gaseous chemical reactant-loaded (gas-loaded) MOF(s).

Handling Fluorinated Gases as Solid Reagents Using Metal-Organic Frameworks. A general strategy for safely handling inexpensive fluorinated gaseous building blocks as benchtop-stable solid reagents using porous metal-organic frameworks (MOFs) was developed. Gas-MOF reagents were employed to facilitate novel fluorovinylation and fluoroalkylation reactions, which represent safe, efficient, and atom-economical alternatives to current methods. Our approach enables high-throughput reaction development with any gaseous reagent, opening the door for the development of myriad new synthetic transformations.

We hypothesized the reversible adsorption of fluorinated gases within porous solids should allow for their facile handling as recyclable solid reagents, overcoming the limitations outlined above and facilitating the development of new methods for fluorinating complex molecules. Among porous solids, metal-organic frameworks (MOFs)—crystalline materials constructed from organic linkers and inorganic nodes—are uniquely modular, allowing for the optimization of storage capacity and enthalpy of adsorption (−ΔH_{ads}) for any gas of interest. In particular, MOFs bearing coordinatively unsaturated metal centers (open metal sites) should reversibly bind synthetically relevant fluorinated gases via strong metal-fluorine (M-F) interactions. Owing to the high theoretical gravimetric and volumetric capacities of MOFs such as Mg₂(dobdc) (dobdc⁴⁻=2,5-dioxidobenzene-1,4-dicarboxylate) (23), 1 mmol (23 mL) of gas can be delivered with as little as 120 mg (0.13 mL) of MOF, resulting in a staggering ˜170-fold volume reduction compared to a free ideal gas (assuming a crystallographic density of 0.909 g/cm³ for Mg₂(dobdc) and one gas molecule per metal site). Herein, we demonstrate that the commodity fluorinated gases VDF, TFP, HFP, and TFMI can be stored within MOFs and handled as solid reagents for the first time, enabling their facile use in otherwise challenging fluoroalkylation and fluorovinylation reactions.

Results and Discussion. Given the scarcity of data available regarding the adsorption of fluorinated commodity chemicals within porous solids, we commenced by studying VDF uptake in twelve representative open metal site MOFs, including HKUST-1 or Cu₃(btc)₂ (btc³⁻=benzene-1,3,5-tricarboxylate), MIL-100(Fe) or Fe₃(O)(OH)(btc)₂, NH₂-MIL-101(Al) or Al₃(O)(OH)(NH₂-bdc)₃ (NH₂-bdc²⁻=2-aminobenzene-1,4-dicarboxylate), MOF-74 or M₂(dobdc) (M=Mg, Mₙ, Fe, Co, Ni, Cu, Zn), and m-MOF-74 or M₂(m-dobdc) (M=Mg, Ni; m-dobdc⁴⁻=4,6-dioxidobenzene-1,3-dicarboxylate). We also investigated one commercially available zeolite bearing accessible Na⁺ sites (Zeolite Y). To identify the optimal material for fluorinated gas delivery, VDF adsorption/desorption isotherms were collected for all materials at 30, 40, and 50° C. Notably, VDF adsorption was found to be fully reversible in every case. The isotherm data were used to determine the gravimetric VDF storage capacities at 30° C., 40° C., and 50° C. under 1 bar of VDF (Table 1). Among the tested materials, Mg₂(dobdc) (7.95 mmol/g, 34 wt %) and Cu₃(btc)₂ (7.64 mmol/g, 33 wt %) possess the highest VDF gravimetric capacities, which is expected given their high densities of open metal sites and low molecular weights. Beyond storage capacity, the enthalpy of adsorption (−ΔH_{ads}) is critical for governing how readily fluorinated gases would be released in situ or under ambient conditions. In order to calculate −ΔH_{ads} values, all adsorption isotherms were fit using Dual-Site Langmuir models. Employing the Clausius-Clapeyron equation, the −ΔH_{ads} values as a function of VDF loading were determined for each material; −ΔH_{ads} values at a loading of 1 mmol/g are shown in Table 5. In general, materials bearing highly Lewis acidic Mg²⁺, Ni²⁺, or Na⁺ sites—namely M₂(dobdc) (M=Mg, Ni), M₂(m-dobdc) (M=Mg, Ni), and Zeolite Y-demonstrate the strongest adsorption of VDF (38-41 kJ/mol). This is likely due in part to the hard-soft acid-base match of hard fluorine atoms with hard metal centers.

Because Mg₂(dobdc), Ni₂(dobdc), and Ni₂(m-dobdc) display high VDF gravimetric storage capacities coupled with strong binding, their ability to adsorb the fluorinated gases TFP, HFP, and TFMI was also evaluated (Tables 2-4, and 6-8). Due to its lower molecular weight, Mg₂(dobdc) adsorbs significantly more of each gas than Ni₂(dobdc) and Ni₂(m-dobdc) at 1 bar and 30° C. In addition, the −ΔH_{ads} values for TFP and TFMI in Mg₂(dobdc) are larger in magnitude than the corresponding values in the Ni-based frameworks, whereas the strength of HFP binding is comparable across all three frameworks. Collectively, the adsorption data suggest that Mg₂(dobdc) is an ideal framework for the storage of fluorinated gases due to its high gravimetric capacity and strong interaction with multiple gases of synthetic interest. Based on the promising features of Mg₂(dobdc), we developed a scalable procedure enabling its synthesis on ˜4 g scale in a single batch.

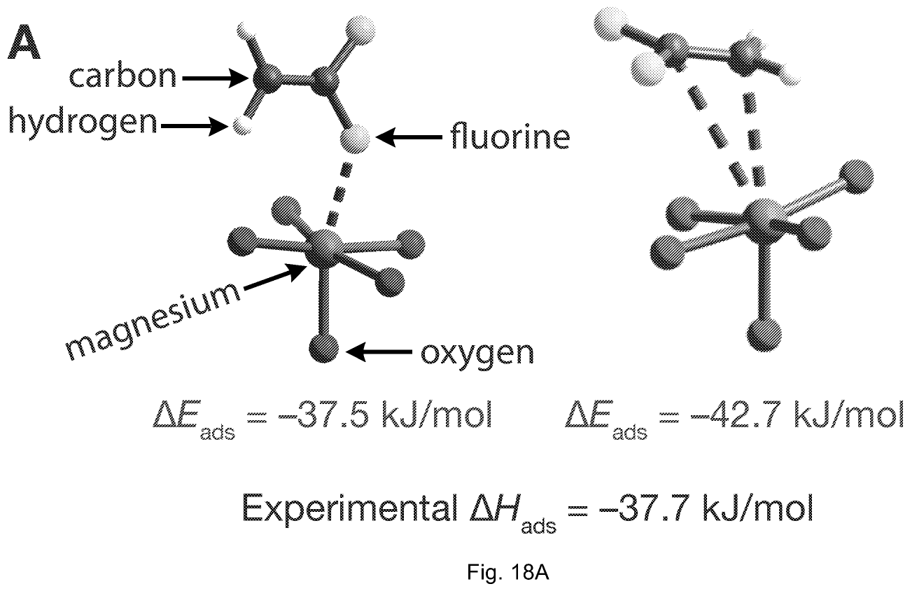

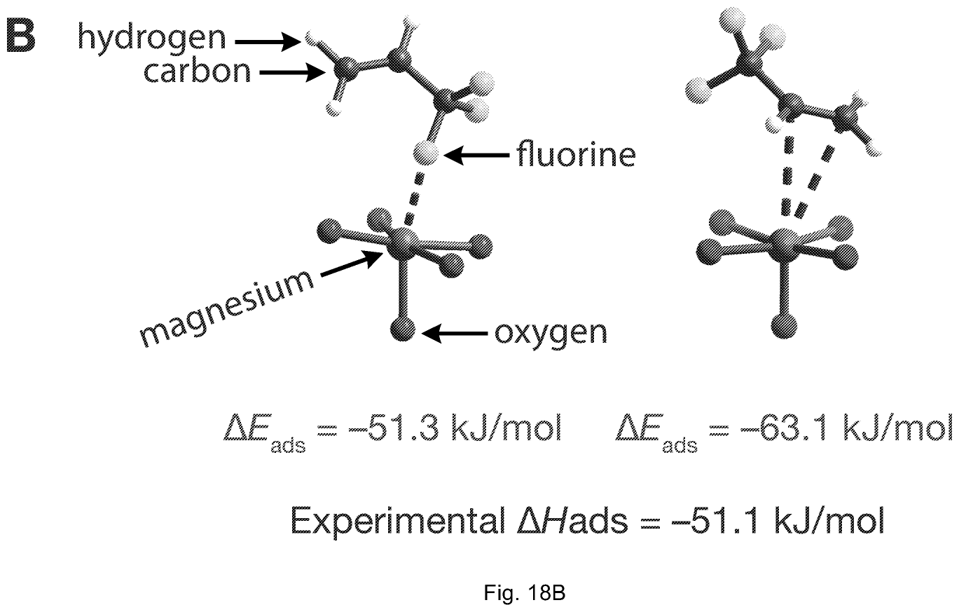

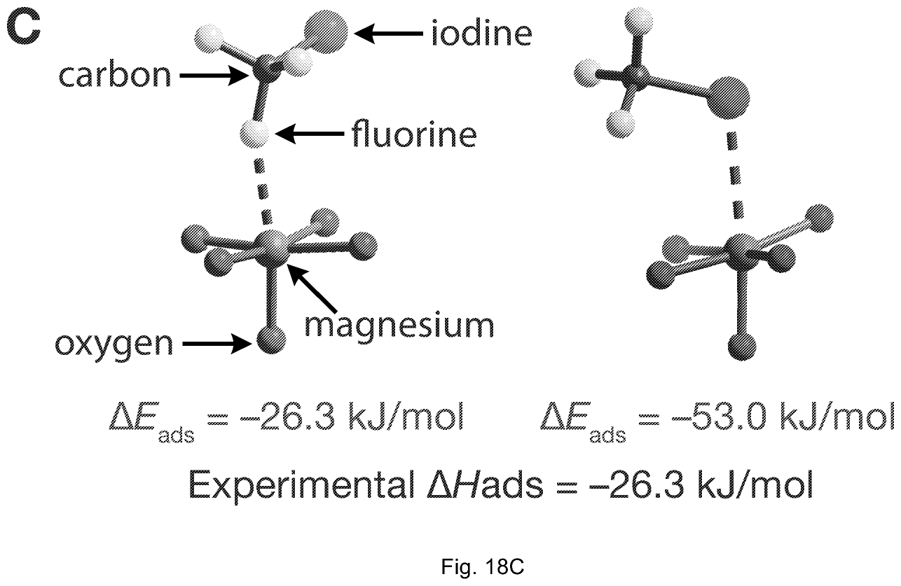

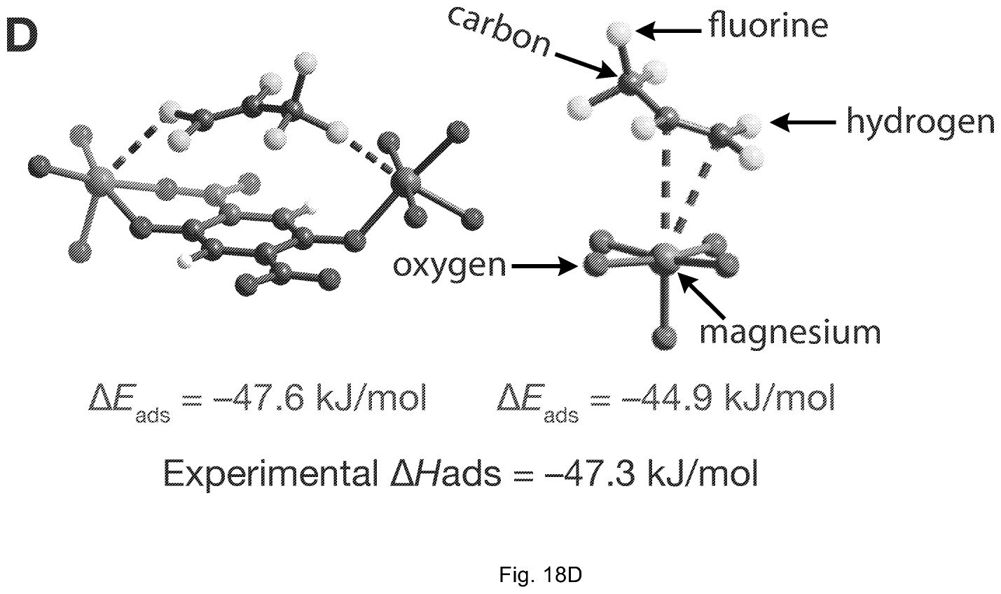

To understand why Mg₂(dobdc) binds a range of fluorinated gases so strongly, we probed the nature of the interaction between Mg₂(dobdc) and VDF (as a representative fluorinated gas) using a range of experimental and computational techniques (Table 9, FIG. 18-20). Magic-angle spinning (MAS)¹⁹F solid-state NMR (SSNMR) measurements were performed on VDF-Mg₂(dobdc). The resonance corresponding to VDF-Mg₂(dobdc) (−87.34 ppm) is shifted upfield and broadened relative to that of VDF dissolved in tetrahydrofuran (THF) (−82.09 ppm). The greater shielding observed for VDF bound within the MOF is due to the proximity of π electron density from the aromatic linkers, and the signal broadening is due to immobilization. The preferred binding mode of VDF in Mg₂(dobdc) was further interrogated via synchrotron powder X-ray diffraction (PXRD) conducted on a sample of microcrystalline Mg₂(dobdc) dosed with ˜100 mbar of VDF. Rietveld refinement of the obtained pattern corroborated a structural model in which VDF preferentially binds to the open Mg²⁺ site through a F . . . Mg interaction, not through the alkene π-bond. The F . . . Mg distance is 2.67(3) Å, which is similar to the distances reported for related experimental and calculated structures. Density-functional theory (DFT) calculations further support that the F-bound structure for VDF-Mg₂(dobdc) possesses a similar predicted binding enthalpy (−ΔH_{b}=37.5 kJ/mol) as the experimental value (−ΔH_{ads}=37.7 kJ/mol) (Table 9, FIG. 18). Indeed, the calculated F-bound structures for TFP, HFP, and TFMI all possess similar predicted binding enthalpies to the experimental values (Table 9, FIG. 18). Overall, these data support that the strong binding of fluorinated gases in Mg₂(dobdc) is due to the favorable interaction between the hard Lewis basic F atoms and the hard Lewis acidic Mg²⁺ open metal sites in Mg₂(dobdc).

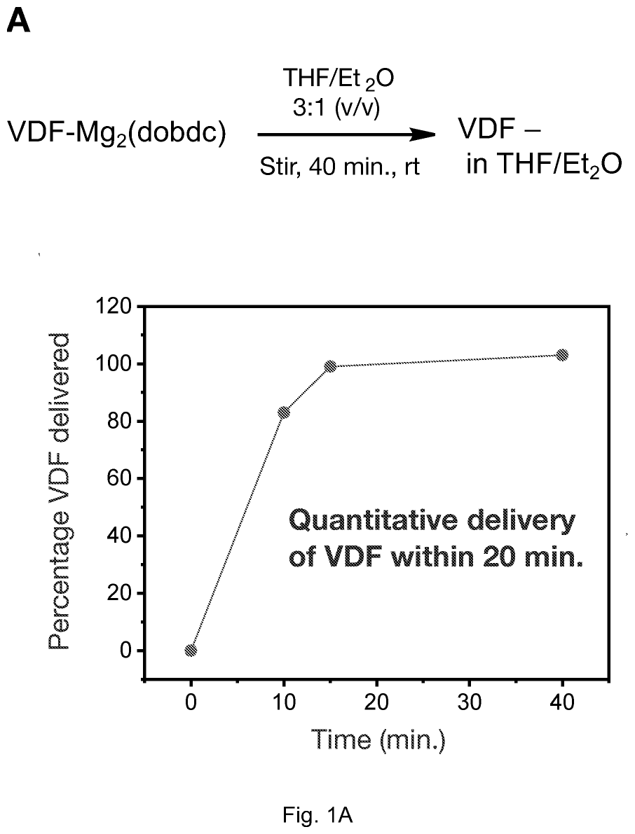

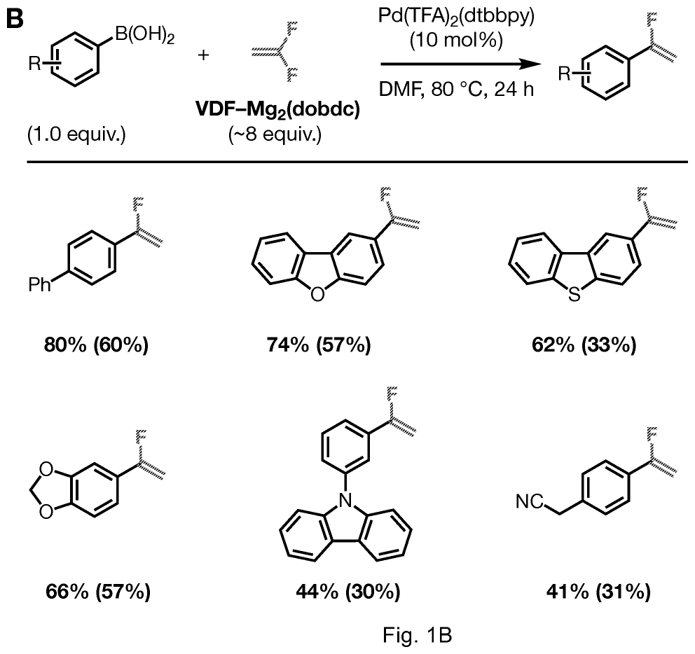

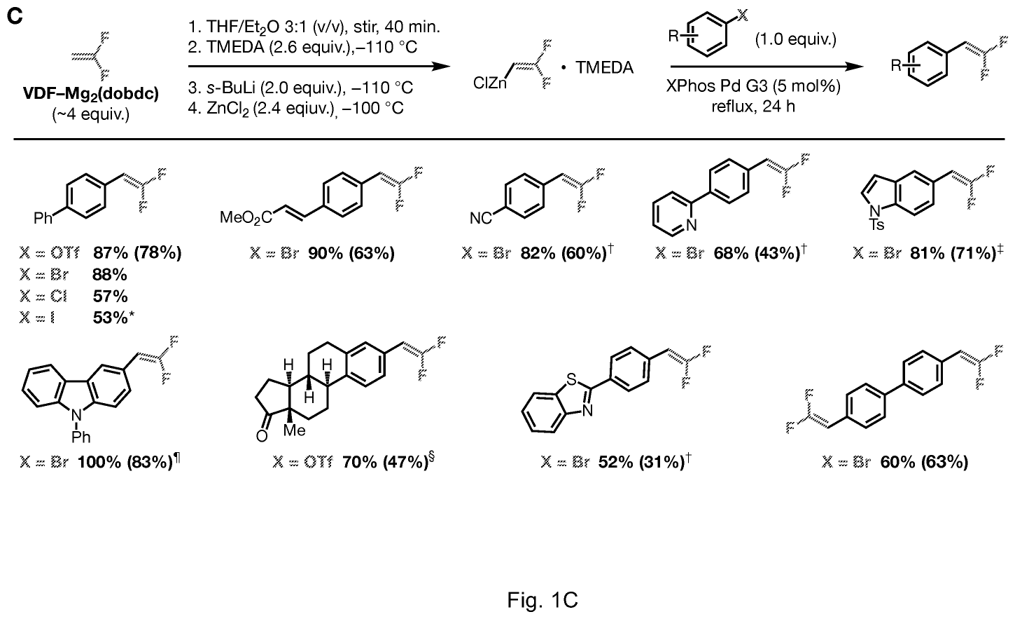

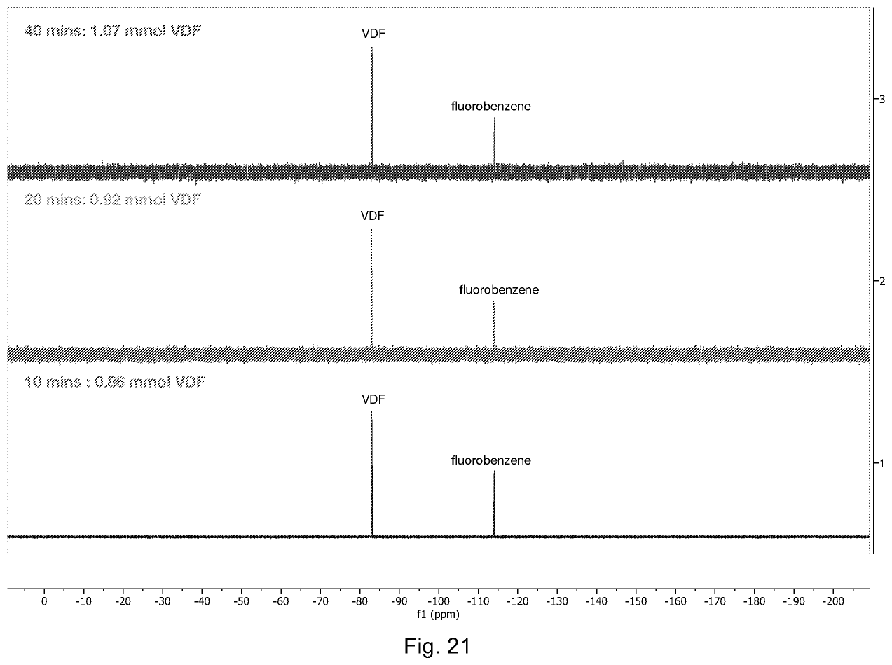

Building upon these results, we investigated whether gas-Mg₂(dobdc) reagents can be used to deliver fluorinated gases under synthetically relevant conditions. Activated Mg₂(dobdc) was dosed with the fluorinated gas, and the resulting gas-MOF reagent was loaded into a custom-built, air-free solid-addition funnel to enable controlled delivery on the benchtop (FIG. 1A). This approach enables 100% of the VDF contained in VDF-Mg₂(dobdc) to be released into a solution of THF and diethyl ether (Et₂O) within 20 minutes (FIG. 1A, FIG. 21), representing a safe, user-friendly delivery system that minimizes gas waste. The mechanism of VDF release likely involves exchange with the Lewis basic solvent. As such, this technique can enable high-throughput screening of reactions involving gases without the need for complex equipment.

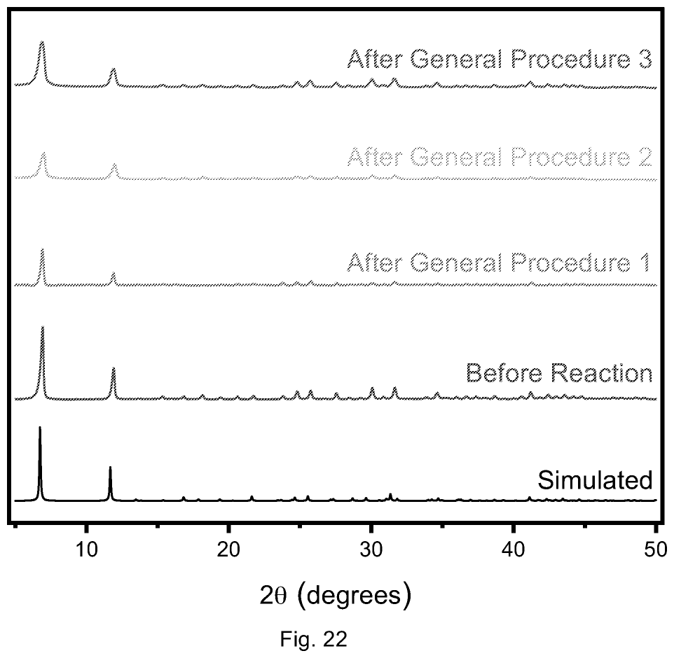

We envisioned that a desirable yet unrealized transformation would be the defluorinative coupling of VDF with easily handled (hetero)aryl nucleophiles to yield valuable α-fluorostyrene products. Due to their topological, electronic, and steric similarities, monofluoroalkenes have been widely utilized as amide bioisosteres in drug and peptidomimetic design. Traditional synthetic routes to access terminal α-fluoroalkenes through alkyne or alkene functionalization pathways suffer from key drawbacks such as multi-step starting material syntheses, harsh/hazardous reaction conditions, poor regioselectivity, and/or modest scopes (36-39). Meanwhile, previously reported metal-catalyzed cross-coupling routes to prepare α-fluorostyrenes require air-sensitive starting materials and/or specialized equipment to handle gaseous VDF. To design a more streamlined protocol, we employed VDF-Mg₂(dobdc) to enable a base-free, defluorinative Suzuki-Miyaura coupling with air-stable (hetero)aryl boronic acids (FIG. 1B), representing a straightforward route to access α-fluorostyrenes. Without the need to handle gaseous reagents, we simply combined 4-biphenylboronic acid and VDF-Mg₂(dobdc) at room temperature (rt) using palladium trifluoroacetate (Pd(TFA)₂) as the catalyst, 4,4′-di-tert-butyl-2,2′-bipyridine (dtbbpy) as the ligand, and N,N-dimethylformamide (DMF) as the solvent to obtain the α-fluorostyrene product in 23% yield (Table 10, entry 1). Although reactions that involve reactive gases are typically low-throughput, using VDF-Mg₂(dobdc) enables the setup of multiple reactions in parallel, streamlining the optimization process (Table 10). An investigation of catalysts, solvents, temperatures, and concentrations revealed that the use of catalytic Pd(TFA)₂(dtbbpy) and a reaction concentration of 50 mM with respect to the arylboronic acid leads to nearly quantitative yield (Table 10, entry 23). Critically, the equivalents of VDF can be easily tuned by adjusting the amount of VDF-Mg₂(dobdc) added to the reaction; doing so revealed that the reaction proceeds well using either 4 or 8 equivalents of VDF (Table 10, entries 23-24). The optimized defluorinative coupling protocol proceeds smoothly with a variety of (hetero)arylboronic acid substrates, furnishing the α-fluorostyrene products in good isolated yields (FIG. 1B). After a successful reaction, the MOF was recovered and found to retain its crystallinity by PXRD, suggesting that it can potentially be recycled if desired (FIG. 22). Using a balloon of VDF in place of VDF-Mg₂(dobdc) affords the product in slightly lower yield (67% compared to 80% with VDF-Mg₂(dobdc)), demonstrating that our strategy is competitive with conventional gas delivery techniques. Given the simplicity of the protocol reported herein, we expect it will find broad use for the synthesis of functionalized α-fluorostyrenes.

Beyond functionalizing the C—F bond of VDF to produce α-fluorostyrenes, we envisaged diversification of the C—H bond to producef β,β-difluorostyrenes as well. Gem-difluoroalkenes are of particular interest in medicinal chemistry because they are bioisosteres for carbonyl groups. For example, the antimalarial activity of artemisinin can be improved by replacing the ester carbonyl group with a gem-difluoroalkene. Standard protocols to install gem-difluoroalkenes via carbonyl olefination reactions or defluorination of trifluoromethylated alkenes involve the use of complex starting materials or reagents and suffer from poor selectivity or modest scopes due to the harsh reaction conditions. The development of cross-coupling methods has led to gem-difluorovinylating reagents including 2,2-difluorovinyl pinacolboranes, 2,2-difluorovinyltributylstannanes, and 2,2-difluorovinyl tosylates. However, these reagents are not bench-stable, and their preparation entails complex synthesis and isolation procedures. As such, access to β,β-difluorostyrene analogues directly from readily available (hetero)aryl halides and VDF represents a desirable transformation. We employed VDF-Mg₂(dobdc) to prepare 2,2-difluorovinylzinc chloride-N,N,N′,N′-tetramethylenediamine (VDF-ZnCl•TMEDA), which can be engaged in Pd-catalyzed Negishi couplings with (hetero)aryl halides to yield β,β-difluorostyrenes (FIG. 1C). A brief reaction optimization revealed that the combination of VDF-ZnCl•TMEDA and catalytic amounts of Xphos Pd G3 in THF/Et₂O at reflux affords the β,β-difluorostyrene products from (hetero)aryl bromides in excellent yields (Table 11, entry 3). Notably, a variety of electrophilic functional groups (esters, nitriles, ketones) are compatible with this protocol, as they do not undergo nucleophilic attack by the organozinc reagent. Further, difluorovinyl groups can be successfully installed onto substrates containing N-heterocycles (pyridine, carbazole, indole, and thiazole) as well as biologically-active molecules (estrone). Finally, this procedure can be extended to achieve a double coupling with an aryl dibromide, producing the bis(difluorovinyl)ated product in good yield. Remarkably, despite exposure to strongly nucleophilic and basic conditions, Mg₂(dobdc) retains its crystallinity after the reaction (FIG. 22). The developed method represents a general approach to preparing β,β-difluorostyrenes under mild conditions.

FIG. 1 shows reactions with VDF. NMR yields were determined by ¹⁹F NMR spectroscopy using fluorobenzene as an internal standard. Isolated yields are shown in parentheses. See supplementary information for experimental details. (A) A sample of VDF-Mg₂(dobdc) in a N₂-filled glovebox was transferred to a custom-built, air-free solid-addition funnel for delivery on the benchtop. The percentage of VDF delivered to a solution of THF/Et₂O over time is shown, assuming a storage capacity of 7.95 mmol VDF per 1.00 g of Mg₂(dobdc). (B) Scope of Pd-catalyzed defluorinative coupling of VDF and (hetero)arylboronic acids using VDF-Mg₂(dobdc). (C) Scope of Negishi coupling of (hetero)aryl halides and VDF-ZnCl•TMEDA synthesized from VDF-Mg₂(dobdc). *Pd(PPh₃)₄, †8 h, 10 mol % Xphos Pd G3, ‡10 mol % Xphos Pd G3, ¶18 h, 10 mol % Xphos Pd G3, § 11 h, 10 mol % Xphos Pd G3. DMF: N,N-dimethylformamide, TFA: trifluoroacetate, dtbbpy: 4,4′-di-tert-butyl-2,2′-dipyridyl, Ph: phenyl, THF: tetrahydrofuran, Et₂O: diethyl ether, Otf: trifluoromethanesulfonate, TMEDA: tetramethylethylenediamine, Me: methyl, Ts: p-toluenesulfonyl.

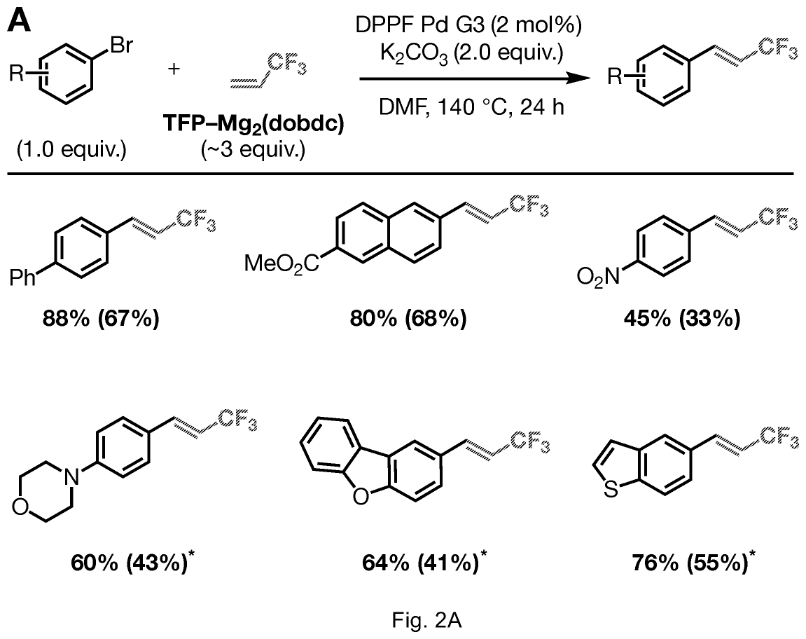

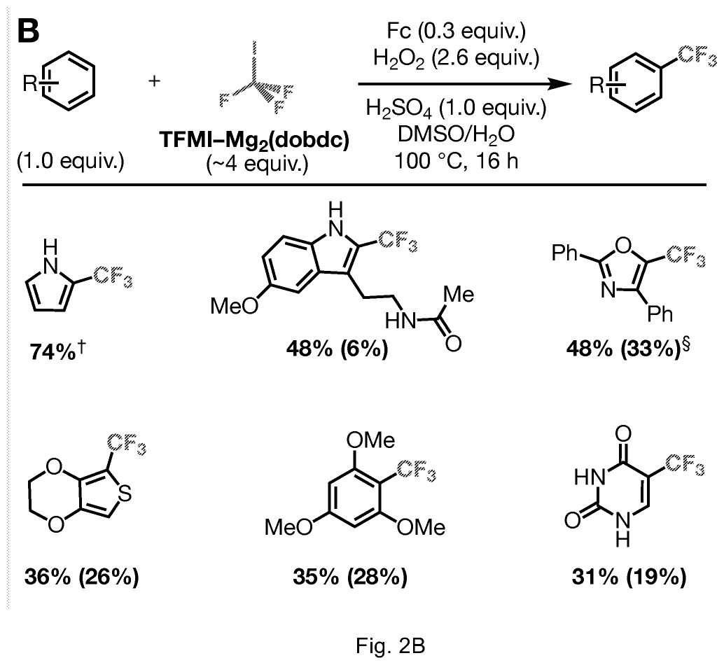

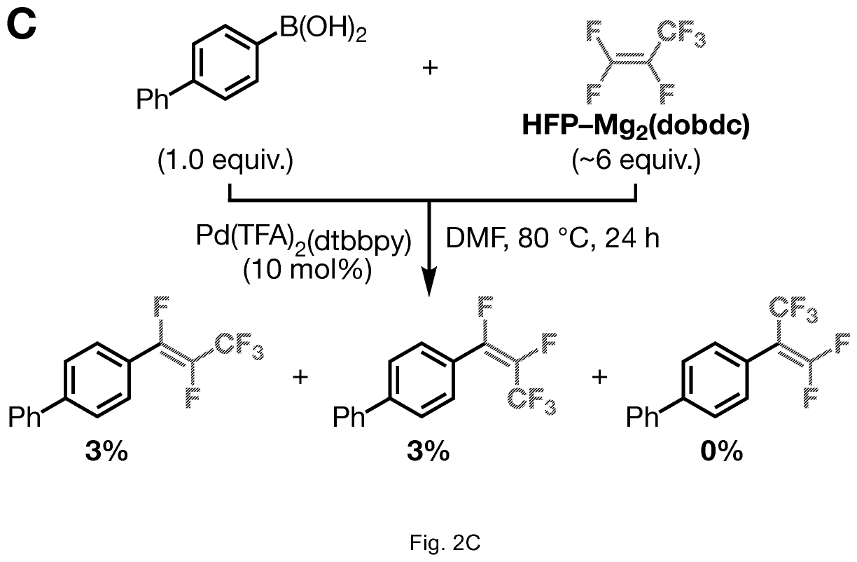

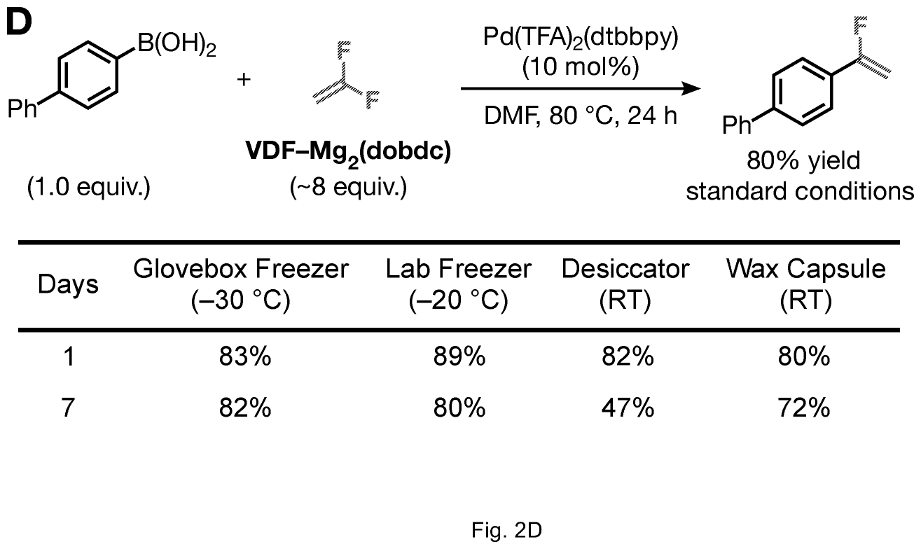

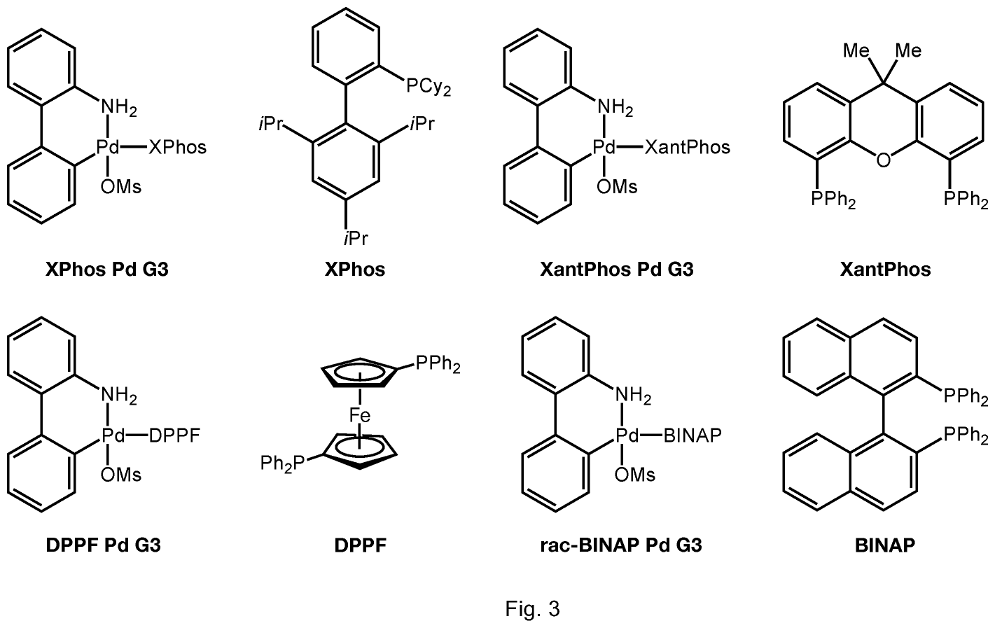

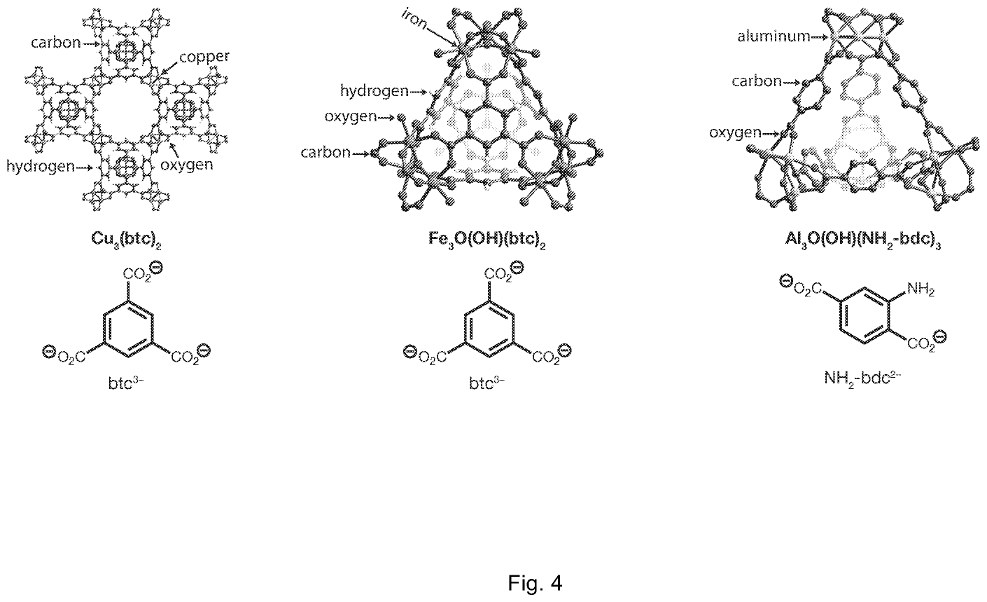

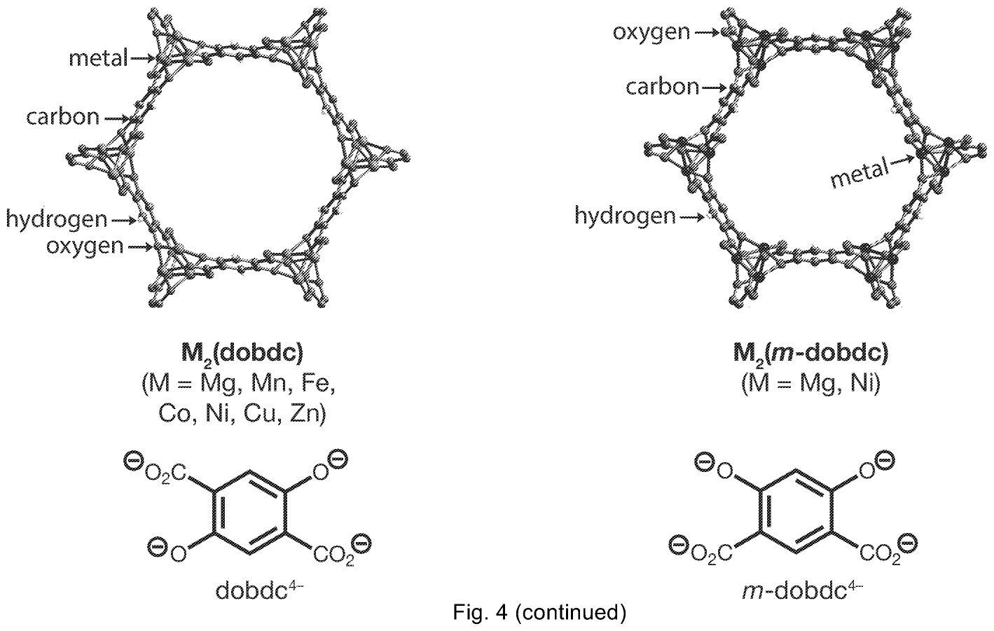

We sought to further explore the generality of our delivery strategy by using Mg₂(dobdc) to deliver other fluorinated gases, namely TFP, TFMI, and HFP. Similar to fluoroalkenes, trifluoromethylated alkenes have been utilized as amide mimics in medicinal chemistry. Conventionally, β-trifluoromethylstyrenes are constructed via the transition metal-catalyzed trifluoromethylation of β-halostyrenes; however, the lack of available β-halostyrene substrates severely limits the reaction scope. Previously reported Pd-catalyzed Heck reactions of TFP and aryl halides suffer from long reaction times, dependence on specialized equipment (e.g., high-pressure reactor autoclaves), the need to generate TFP in situ, and/or limited scopes (15, 66). Building upon these precedents, we employed TFP-Mg₂(dobdc) reagents to deliver TFP for Heck coupling reactions with electronically diverse (hetero)aryl bromides to access a broad scope of f-trifluoromethylstyrenes (FIG. 2A). We investigated the Pd-catalyzed reaction of 4-bromobiphenyl with TFP under standard Heck coupling conditions employing palladium acetate (Pd(Oac)₂) as the catalyst, potassium carbonate (K₂CO₃) as the base, and DMF as the solvent at 150° C., which affords the f-trifluoromethylstyrene product in 17% yield (Table 12, entry 1). During an investigation of a variety of Pd catalysts, we found that the use of DPPF Pd G3 leads to synthetically useful yields (Table 12, entry 3). Although electron-neutral and -deficient aryl bromides react efficiently under these conditions, electron-rich derivatives exhibit sluggish reactivity. The simplicity of working with TFP-Mg₂(dobdc) enabled us to quickly survey different Pd catalysts and discover that the use of XantPhos Pd G3 in conjunction with tetrabutylammonium bromide (TBAB) as a phase transfer catalyst leads to improved yields for these challenging substrates (Table 13, entry 6). With optimized conditions in hand, the reaction scope was expanded to an array of (hetero)aryl bromides bearing a variety of functional groups, furnishing the β-trifluoromethylstyrene products in good yields (FIG. 2A).

We next employed TFMI-Mg₂(dobdc) reagents to introduce valuable trifluoromethyl groups into (hetero)aromatic compounds (FIG. 2B). Trifluoromethyl groups are ubiquitous in drugs, representing ˜19% of fluorinated pharmaceuticals. TFMI represents an inexpensive and safe reagent for generating trifluoromethyl radicals. Compared to traditional Ru- or Ir-based photoredox catalysis, Fe salts are inexpensive potential catalysts for oxidatively generating trifluoromethyl radicals from TFMI without the need for complicated reaction setups or light irradiation. As such, we focused on developing a general procedure for radical Minisci-like (hetero)arene trifluoromethylation using Fe catalysts. A brief optimization using uracil as the model substrate revealed that the combination of TFMI-Mg₂(dobdc), ferrocene, hydrogen peroxide (H₂O₂), and sulfuric acid (H₂SO₄) in dimethyl sulfoxide (DMSO) and H₂O at 100° C. affords the trifluoromethylated product in good yield (Table 14, entry 3). These conditions can be applied to a variety of substrates, including five-membered heteroarenes (pyrrole, indole, oxazole) and biologically active molecules (melatonin and uracil). Although the major product is accompanied by trifluoromethylation at other positions, isolation of the desired isomer in every case is possible via flash chromatography.

Scattered reports in the literature suggest that the Pd-catalyzed coupling of HFP with aryl magnesium bromides and arylboronic acid neopentylglycol esters result in mixtures of stereo- and regioisomers. We sought to expand the scope of our defluorinative coupling of VDF and commercially available (hetero)arylboronic acids (FIG. 1B) to achieve a similar reaction using HFP-Mg₂(dobdc). Our preliminary efforts led to the defluorinative coupling of HFP and 4-biphenylboronic acid to furnish a 1:1 E:Z mixture of pentafluoropropene-substituted products (FIG. 2C). This result demonstrates how the use of gas-MOF reagents enables reaction development with multiple gases without the need to change cylinders attached to complicated manifolds.

FIG. 2 shows reactions with other fluorinated gases. NMR yields were determined by ¹⁹F NMR spectroscopy using fluorobenzene as an internal standard. Isolated yields are shown in parentheses. See supplementary information for experimental details. (A) Scope of Pd-catalyzed Heck coupling of (hetero)aryl bromides and TFP using TFP-Mg₂(dobdc). *XantPhos Pd G3 (2 mol %) and tetrabutylammonium bromide (1 equiv.). (B) Scope of Fe-catalyzed trifluoromethylation of (hetero)arenes using TFMI-Mg₂(dobdc). †0.15 mmol FeSO₄·7H₂O, 50° C., § 50° C. Fc: ferrocene, DMSO: dimethyl sulfoxide (C) Pd-catalyzed defluorinative coupling of HFP and arylboronic acids using HFP-Mg₂(dobdc). (D) User-friendly variations of defluorinative couplings of VDF and arylboronic acids using VDF-Mg₂(dobdc).

Throughout the reaction development outlined above, we avoided exposing the gas-Mg₂(dobdc) reagents to air to prevent gas loss. To gauge the storability of the gas-Mg₂(dobdc) reagents, their stability under ambient conditions was evaluated. We selected the defluorinative coupling of VDF and 4-biphenylboronic acid as a model reaction and investigated whether exposing the VDF-Mg₂(dobdc) reagent briefly to air before adding it to the reaction mixture results in VDF leakage. Exposing freshly prepared VDF-Mg₂(dobdc) briefly to air before adding it to the reaction (Table 15, entry 2) produces the same yield as the standard conditions (Table S15, entry 1). This result demonstrates that the gas-Mg₂(dobdc) reagents can be handled on the benchtop with negligible loss in performance. To further gauge the storability of the gas-Mg₂(dobdc) reagents, we evaluated the performance of the defluorinative coupling of VDF and 4-biphenylboronic acid after storing the VDF-Mg₂(dobdc) under different conditions for up to 7 days. First, we found that VDF-Mg₂(dobdc) can be stored in a vial at −30° C. under an inert atmosphere for up to 7 days before adding it to the reaction on the benchtop with negligible loss in performance (FIG. 2D, left column). Moreover, leaving VDF-Mg₂(dobdc) at −20° C. for as long as 7 days in air before adding it to the reaction does not hinder product formation (FIG. 2D, mid-left column). Storage of VDF-Mg₂(dobdc) in a desiccator at RT for one day before adding it to the reaction results in 82% yield, whereas storage in a desiccator for up to 1 week still affords the product in a moderate yield (FIG. 2D, mid-right column). Leaving a vial of VDF-Mg₂(dobdc) on the benchtop at RT overnight before adding it to the reaction also produces a good yield of product (Table 15, entry 3). Together, these results suggest that gas loss from VDF-Mg₂(dobdc) is largely driven by entropic effects and that gas-MOF reagents should be stable under ambient conditions as long as they are stored at low temperatures. In order to further improve the long-term benchtop stability of the gas-MOF reagents at RT, we packed VDF-Mg₂(dobdc) into wax capsules to prevent contact with water and minimize gas loss. Storage of VDF-Mg₂(dobdc) in a wax capsule on the benchtop at RT for seven days before adding it to the reaction results in good yield of the product (FIG. 2D, right column). Ideally, storage in wax capsules will enable the gas-MOF reagents to be stored in air at RT for extended periods of time with minimal gas loss. Intriguingly, a wax capsule packed with VDF-Mg₂(dobdc) can be suspended in a solution of DMF for at least 60 minutes without releasing VDF-Mg₂(dobdc) or free VDF to solution (Table 18), suggesting that the wax capsule is not permeable to VDF. Sonicating the reaction mixture breaks open the capsule and enables controlled release of VDF to solution at the desired time (Table 18).

Collectively, we have demonstrated that fluorinated gases can be handled as free-flowing solids and released under synthetically relevant conditions using MOFs to streamline a series of novel fluorovinylation and trifluoromethylation reactions. While we focus on fluorinated gases, in theory this strategy can be generalized to the delivery of any gaseous reagent of interest to synthetic organic and medicinal chemists. We expect that these gas-MOF reagents will streamline the optimization of many more transformations involving gaseous reagents, which remain frequently overlooked by the synthetic community due to the challenges associated with their safe handling in the laboratory.

Materials and Methods. All reagents were purchased from commercial vendors and used without additional purification unless specified otherwise. 4,6-dihydroxy-1,3-benzenedicarboxylic acid was prepared according to a previous literature procedure. Palladium acetate (Pd(Oac)₂), tetrakis(triphenylphosphine)palladium (Pd(PPh₃)₄), and bis(triphenylphosphine)palladium dichloride ((PPh₃)₂PdCl₂) were purchased from Aldrich. Palladium trifluoroacetate (Pd(TFA)₂) was purchased from Alfa Aesar. Xphos Pd G3 precatalyst, XantPhos Pd G3 precatalyst, BINAP Pd G3 precatalyst, and DPPF Pd G3 were prepared according to a previous literature procedure. Bis(tricyclohexylphosphine)nickel dichloride ((Pcy₃)₂NiCl₂) was purchased from TCI America. The purity of (hetero)arylboronic acid starting materials was verified by ¹H NMR before use. The 4-biphenylboronic acid (Oakwood Chemical) was rinsed with dichloromethane (CH₂Cl₂) on a fritted funnel to remove biphenyl contaminants until the starting material was pure, as determined by thin-layer chromatography (TLC). Anhydrous, oxygen-free tetrahydrofuran (THF), diethyl ether (Et₂O), N,N-dimethylformamide (DMF), and toluene were obtained by vigorously sparging with argon for 30 min, followed by passage through two columns of activated alumina using a Phoenix SDS JC Meyer Solvent System. All other anhydrous, oxygen-free solvents were purchased from Aldrich in Sure-Seal™ bottles and sparged with N₂ before use. The methanol (MeOH) that was used to synthesize Mn₂(dobdc) was dried over 3 Å molecular sieves and sparged with N₂ before use. All procedures were carried out on the benchtop unless specified otherwise.

Solution-state NMR data were collected on a Bruker INOVA 400 MHz or a Bruker INOVA 500 MHz spectrometer and are referenced to residual solvent. Magic angle spinning (MAS)¹⁹F solid-state NMR (SSNMR) measurements were carried out using a Phoenix NMR HX NB Probe (3.2 mm) located within a Varian INOVA 500 MHz spectrometer. All ¹⁹F NMR yields stated for fluorination reactions are calculated from ¹⁹F NMR spectra relative to an internal standard of fluorobenzene. All MAS SSNMR experiments were carried out using samples packed within 35 μL rotors at a spinning speed of 20 kHz. Infrared spectra were collected on a Bruker Tensor II IR spectrometer with a diamond Attenuated Total Reflectance (ATR) attachment. High-resolution mass spectrometry (HRMS) data were collected on an Exactive Orbitrap mass spectrometer (Thermo Scientific) equipped with a DART ion source (IonSense Inc.). Melting points were determined by placing compounds in capillary tubes (CG-1841-01) and using a REACH Devices digital melting point determination apparatus (RD-MP). Powder X-ray diffraction (PXRD) patterns were collected on a Rigaku Ultima IV diffractometer equipped with a CuKα source (k=1.54 Å) and were baseline corrected after data collection using OriginPro. Flash chromatography was performed on a Biotage Isolera One system using silica gel 60 adsorbent for chromatography (Lancaster Synthesis).

Surface area data were collected on a Micromeritics 3-flex gas sorption analyzer or a Micromeritics ASAP 2020 using ultrapure N₂ (99.999%) and a liquid N₂ bath. Brunauer-Emmett-Teller (BET) and Langmuir surface areas were determined by linear least squares regression analysis using the linearized forms of the BET and Langmuir equations, respectively. Fluorinated gas adsorption isotherms were collected on a Micromeritics 3-flex gas sorption analyzer. A circulating water bath was employed to maintain the temperature during isothermal measurements.

Fluorinated gas adsorption data were fit using the dual-site Langmuir model (Equation 1), where Q(P) is the predicted uptake Q at pressure P in mmol/g, Q_{sat,i} is the saturation pressure of binding site i in mmol/g, bᵢ is the Langmuir parameter of site i, vᵢ is the Freundlich parameter of site i, —Si is the entropy of binding site i in J/mol•K, R is the ideal gas constant, Eᵢ is the enthalpy of adsorption for binding site i in kJ/mol, and T is the temperature in K. The isotherms were fit with v₁ and v₂ set as 1. Fits were obtained using Solver in Microsoft Excel.

$$Q ( P ) = \frac{{{Q}_{sat 1} ( {b}_{1} P )}^{{v}_{1}}}{1 + {( {b}_{1} P )}^{{v}_{1}}} + \frac{{{Q}_{sat 2} ( {b}_{2} P )}^{{v}_{2}}}{1 + {( {b}_{2} P )}^{{v}_{2}}} ; {b}_{i} = {e}^{( \frac{- {S}_{i}}{R} ) ( \frac{1000 \cdot {E}_{i}}{R} )} \qquad Equation 1$$

Heats of adsorption (−ΔH_{ads}) values were calculated using the Clausius-Clapeyron equation (Equation 2), where PQ are pressure values corresponding to the same loading Q, ΔH_{ads} is the differential enthalpy of adsorption in kJ/mol, R is the ideal gas constant, T is the temperature in K, and c is a constant. Fits over a range of Q values were obtained using Mathematica to calculate the differential enthalpies of adsorption (ΔH_{ads}) based on the slopes of the linear trendlines fit to ln(PQ) vs 1/T at constant values of Q.

$$Ln ( {P}_{Q} ) = ( \frac{Δ {H}_{ads}}{R} ) ( \frac{1}{T} ) + c \qquad Equation 2$$

Metal-Organic Frameworks Investigated. Synthesis and Characterization of Metal-Organic Frameworks.

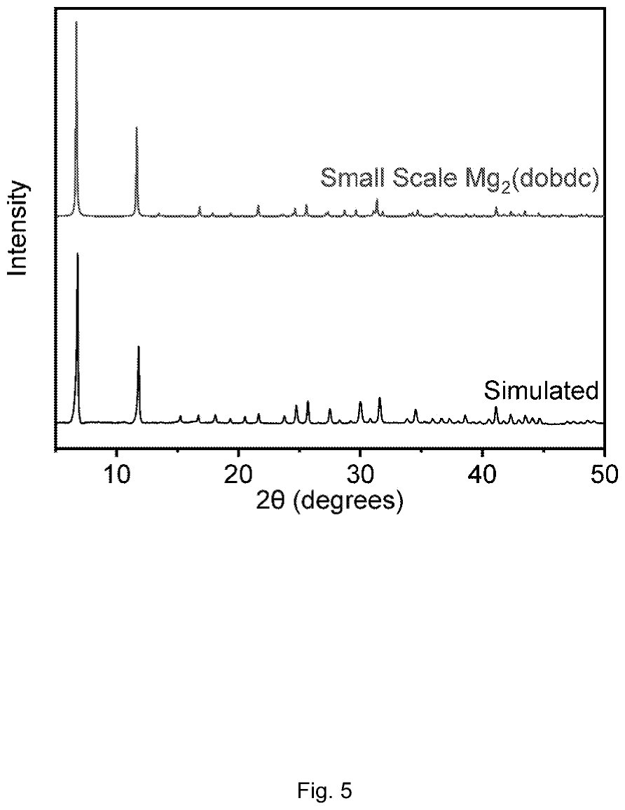

Mg₂(dobdc). Prepared on small scale for gas sorption measurements according to a previous literature procedure. A 1 L round bottom flask was charged with Mg(NO₃)₂•6H₂O (3.21 g, 12.5 mmol, 2.50 equiv.), 2,5-dihydroxyterephthalic acid (0.991 g, 5.00 mmol, 1.00 equiv.), deionized water (25 mL), fresh DMF (450 mL), and ethanol (EtOH, 25 mL). The mixture was sonicated until all of the solids were dissolved. The reaction mixture was allowed to stir slowly, refluxing at 120° C. for 24 h, resulting in the precipitation of a yellow powder from solution. The reaction mixture was allowed to cool to room temperature and filtered. The solid was quickly transferred to a 350 mL screw-cap high pressure reaction vessel filled with fresh DMF (150 mL). The reaction vessel was placed in a silicone oil bath that had been pre-heated to 120° C. and allowed to stand at 120° C. for 24 h. At this time, the heterogeneous mixture was allowed to cool to room temperature and filtered. The solid was returned to the reaction vessel with fresh DMF (150 mL) and returned to a silicone oil bath that had been pre-heated to 120° C. This soaking process was repeated for a total of three DMF soaks. The mixture was then filtered, and the solid was transferred to a 350 mL screw-cap high pressure reaction vessel filled with methanol (MeOH, 150 mL). The reaction vessel was placed in a silicone oil bath that had been pre-heated to 60° C. and allowed to stand at 60° C. for 24 h. At this time, the heterogeneous mixture was filtered, and the solid was returned to the reaction vessel filled with fresh MeOH (150 mL). The vessel was transferred to a silicone oil bath that had been pre-heated to 60° C. and allowed to stand at this temperature for 24 h. At this time, the heterogeneous mixture was filtered, and the solid was transferred to the reaction vessel filled with fresh MeOH (150 mL). This soaking process was repeated for a total of three MeOH soaks. The mixture was filtered again, and the solid was quickly transferred to a Schlenk flask under N₂. The material was activated under flowing N₂ at 180° C. for 24 h, and then by heating under high vacuum (<100 mTorr) at 180° C. for 24 h. The Schlenk flask was transferred into a N₂-filled glovebox, and the solid was transferred to a glass adsorption tube equipped with a Micromeritics TransSeal. The tube was removed from the glovebox and the material was activated for an additional 48 h under high vacuum (<10 pbar) at 180° C. Activated Mg₂(dobdc) was obtained as a golden yellow solid. Langmuir surface area determined from the 77 K N₂ adsorption isotherm: 1912±9 m²/g (Lit. 1957 m²/g). The PXRD pattern (CuKα radiation, k=1.5418 Å) of MeOH-solvated Mg₂(dobdc) is given in FIG. 5.

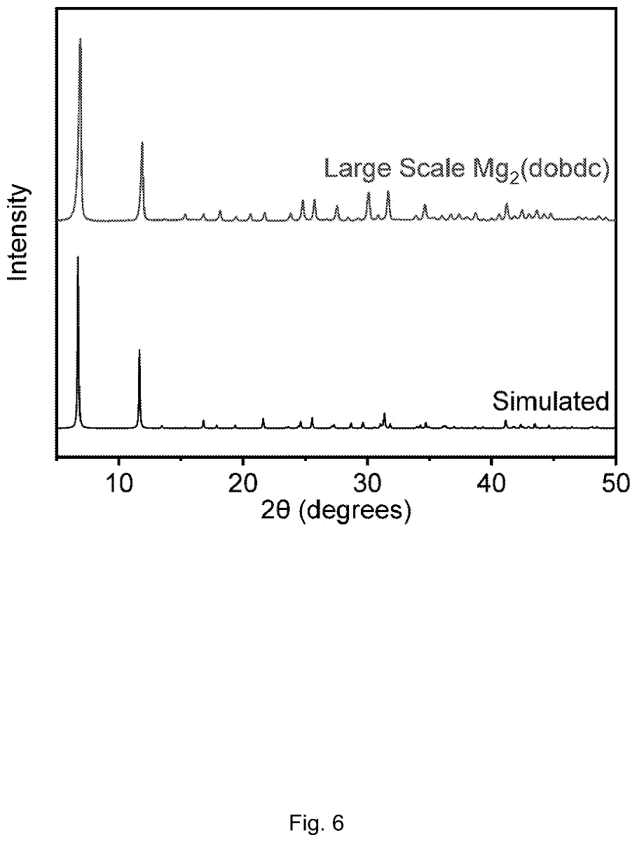

Large-scale synthesis of Mg₂(dobdc). In order to prepare Mg₂(dobdc) on large scale, a new high-concentration synthesis procedure was developed. A 150 mL screw-cap high-pressure flask equipped with a stir bar was charged with Mg(NO₃)₂•6H₂O (12.8 g, 50.0 mmol, 2.50 equiv.), 2,5-dihydroxyterephthalic acid (3.96 g, 20.0 mmol, 1.00 equiv.), DMF (18 mL), EtOH (1.0 mL), and water (1.0 mL). The flask was capped and placed in a silicone oil bath, and the reaction mixture was heated to 120° C. while stirring vigorously (1000 rpm). The reaction mixture was allowed to stir at 120° C. for 24 h. At this time, the reaction mixture was allowed to cool to room temperature and filtered. The resulting tan solid was rinsed thoroughly with DMF (100 mL). The solid was transferred to a 1 L screw-cap high-pressure flask and DMF (500 mL) was added. The pressure flask was sealed with parafilm, and the mixture was sparged with N₂ for 1 h. At this time, the parafilm was removed and the pressure vessel was quickly capped. The reaction mixture was allowed to stir at reflux for 120 h. Upon full conversion of the solid to Mg₂(dobdc) (as confirmed by PXRD), the heterogeneous reaction mixture was allowed to cool to room temperature and filtered. The solid was transferred to a 1 L Pyrex jar filled with MeOH (900 mL). The jar was transferred to an oven that had been pre-heated to 60° C. and allowed to stand at this temperature for 24 h. At this time, the heterogenous mixture was allowed to cool to room temperature, the solvent was decanted, and fresh MeOH (900 mL) was added. The jar was returned to the oven that had been pre-heated to 60° C. This process was repeated for a total of eight MeOH soaks. The mixture was filtered, and the solid was transferred to a Schlenk flask. The sample was activated on the Schlenk line under flowing N₂ for 6 h at room temperature and then under high vacuum (<100 mTorr) while ramping the temperature slowly to 300° C. (0.2° C./min) in a sand bath. The sample allowed to stand at 300° C. under high vacuum (<100 mTorr) for 12 h. At this time, the Schlenk flask was transferred into a N₂-filled glovebox, yielding activated Mg₂(dobdc) (4.233 g, 87% yield) that was stored in a N₂-filled glovebox when not in use. A portion (−100 mg) of Mg₂(dobdc) was directly transferred to a glass adsorption tube equipped with a Micromeritics CheckSeal. The sample was activated on the gas sorption analyzer under highs vacuum (<10 pbar), ramping the temperature slowly to 300° C. (0.2° C./min). The sample allowed to stand at 300° C. under high vacuum (<10 pbar) for 12 h prior to gas adsorption analysis. BET surface area determined from the 77 K N₂ adsorption isotherm: 1728 m²/g±2 m²/g (Lit. 1510 m²/g) (6). Langmuir surface area determined from the 77 K N₂ adsorption isotherm: 2141 m²/g±26 m²/g (Lit. 1957 m²/g). The PXRD pattern (CuKα radiation, k=1.5418 Å) of MeOH-solvated Mg₂(dobdc) is given in FIG. 6.

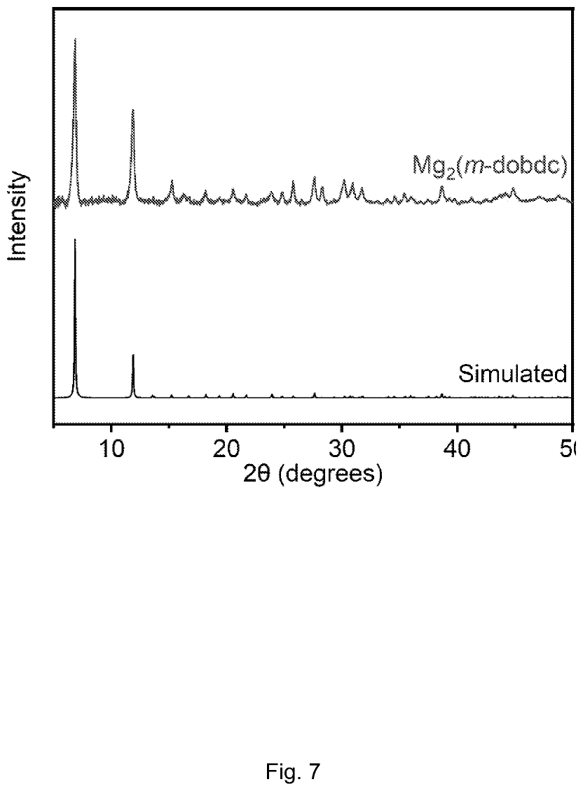

Mg₂(m-dobdc). Prepared according to a previous literature procedure. A 150 mL screw-cap high-pressure flask equipped with a stir bar was charged with Mg(NO₃)₂•6H₂O (300. Mg, 1.17 mmol, 2.49 equiv.), 4,6-dihydroxy-1,3-benzenedicarboxylic acid (93.0 mg, 0.469 mmol, 1.00 equiv.), DMF (31 mL), MeOH (14 mL), and water (1.0 mL). The flask was capped and placed in a silicone oil bath that had been pre-heated to 120° C. The reaction mixture was allowed to stir at 120° C. for 8 h. At this time, the reaction mixture was allowed to cool to room temperature and filtered. The solid was transferred to a 100 mL Pyrex jar filled with DMF (50 mL). The jar was transferred to an oven that had been pre-heated to 120° C. and allowed to stand at this temperature for 24 h. At this time, the heterogeneous mixture was allowed to cool to room temperature, the solvent was decanted, and fresh DMF (50 mL) was added. The jar was returned to the oven that had been pre-heated to 120° C. This process was repeated for a total of three DMF soaks. After the third DMF wash, the solid was filtered and returned to the jar with fresh methanol (50 mL). The jar was transferred to an oven that had been pre-heated to 60° C. and allowed to stand at this temperature for 24 h. At this time, the heterogenous reaction mixture was allowed to cool to room temperature, the solvent was decanted, and fresh MeOH (50 mL) was added. The jar was returned to the oven that had been pre-heated to 60° C. This process was repeated for a total of three MeOH soaks. The mixture was filtered, and the solid was transferred to a Schlenk flask. The sample was activated on the Schlenk line under high vacuum (<100 mTorr) at 180° C. for 24 h. The Schlenk flask was transferred into a N₂-filled glovebox, and the solid was transferred to a glass adsorption tube equipped with a Micromeritics TransSeal. The tube was removed from the glovebox, and the material was activated for an additional 24 h under high vacuum (<10 pbar) at 180° C. Activated Mg₂(m-dobdc) was obtained as a light brown solid. Langmuir surface area determined from the 77 K N₂ adsorption isotherm: 1882±32 m²/g (Lit. 1793 m²/g) (8). The PXRD pattern (CuKα radiation, k=1.5418 Å) of MeOH-solvated Mg₂(m-dobdc) is given in FIG. 7.

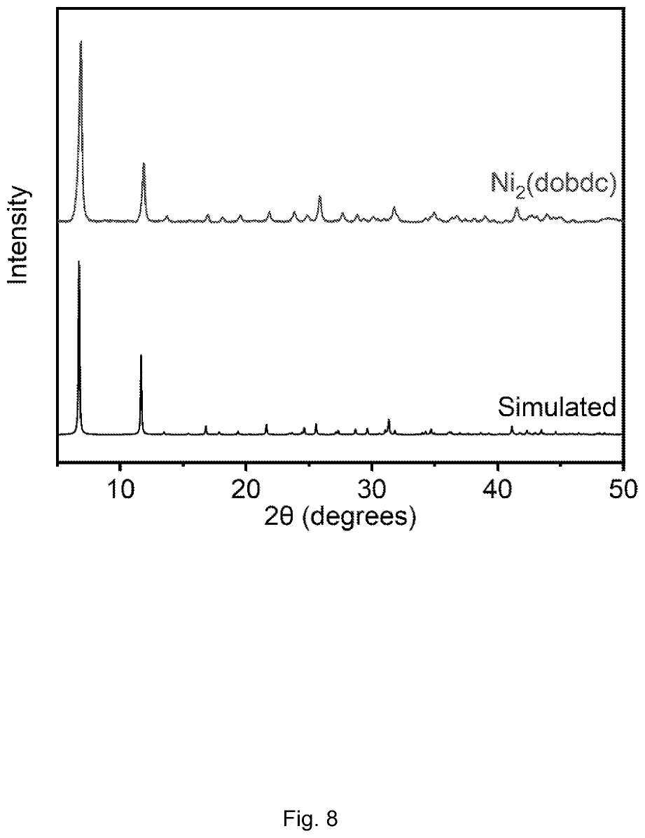

Ni₂(dobdc). Prepared according to a previous literature procedure. A 350 mL screw-cap high pressure reaction vessel equipped with a stir bar was charged with Ni(NO₃)₂•6H₂O (5.23 g, 18.0 mmol, 2.51 equiv.), 2,5-dihydroxyterephthalic acid (1.42 g, 7.17 mmol, 1.00 equiv.), fresh DMF (175 mL), and MeOH (21 mL). The mixture was sonicated until all of the solids were dissolved. The reaction mixture was vigorously sparged with N₂ for 1 h. The reaction vessel was capped and transferred to a silicone oil bath. The reaction mixture was heated to 120° C. and allowed to stir slowly at 120° C. for 14 h, resulting in the precipitation of a yellow-brown solid from solution. The heterogeneous reaction mixture was allowed to cool to room temperature and filtered. The solid was quickly transferred to a 500 mL Pyrex jar filled with DMF (250 mL). The jar was placed in an oven that had been pre-heated to 100° C. and allowed to stand at 100° C. for 24 h. At this time, the heterogeneous mixture was allowed to cool to room temperature and filtered. The solid was returned to the jar with fresh DMF (250 mL) and returned to an oven that had been pre-heated to 100° C. This soaking process was repeated for a total of three DMF soaks. The mixture was then filtered and transferred to a 500 mL Pyrex jar filled with MeOH (250 mL). The jar was placed in an oven that had been pre-heated to 60° C. and allowed to stand at 60° C. for 24 h. At this time, the heterogeneous mixture was allowed to cool to room temperature and filtered. The solid was returned to the jar with fresh MeOH (250 mL) and returned to an oven that had been pre-heated to 60° C. This soaking process was repeated for a total of three MeOH soaks. The mixture was filtered again, and the solid was quickly transferred to a Schlenk flask under N₂. The material was activated under flowing N₂ at 180° C. for 24 h, and then by heating under high vacuum (<100 mTorr) at 180° C. for 24 h. The Schlenk flask was transferred into a N₂-filled glovebox, and the solid was transferred to a glass adsorption tube equipped with a Micromeritics TransSeal. The tube was removed from the glovebox and the material was activated for an additional 24 h under high vacuum (<10 pbar) at 180° C. Activated Ni₂(dobdc) was obtained as a yellow solid. Langmuir surface area determined from the 77 K N₂ adsorption isotherm: 1406 m²/g±46 m²/g (Lit. 1574 m²/g). The PXRD pattern (CuKα radiation, k=1.5418 Å) of MeOH-solvated Ni₂(dobdc) is given in FIG. 8.

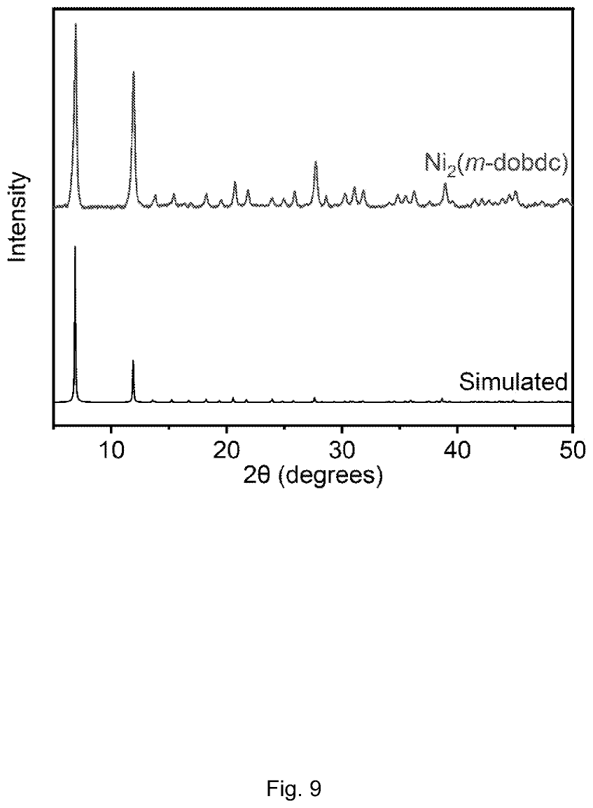

Ni₂(m-dobdc). Prepared according to a literature procedure. A 350 mL screw-cap high-pressure reaction flask equipped with a stir bar was charged with NiCl₂ (1.17 g, 9.00 mmol, 2.50 equiv.), 4,6-dihydroxy-1,3-benzenedicarboxylic acid (720 mg, 3.60 mmol, 1.00 equiv.), DMF (156 mL), and MeOH (84 mL). The reaction flask was sealed and transferred to a silicone oil bath. The oil bath was heated to 120° C., and the reaction mixture was stirred gently (300 rpm) at this temperature for 24 h. Over the course of the reaction, a bright green solid precipitated from solution. The reaction mixture was allowed to cool to room temperature, filtered, and washed with DMF (200 mL). The solid was transferred to a 500 mL Pyrex jar filled with DMF (200 mL). The jar was transferred to an oven that had been pre-heated to 120° C. and allowed to stand at this temperature for 24 h. At this time, the solvent was decanted, and fresh DMF (200 mL) was added. The jar was returned to the oven that had been pre-heated to 120° C. This process was repeated for a total of three DMF soaks. After the third DMF wash, the solid was filtered and returned to the jar with fresh methanol (200 mL). The jar was transferred to an oven that had been pre-heated to 60° C. and allowed to stand at this temperature for 24 h. At this time, the heterogeneous mixture was allowed to cool to room temperature, the solvent was decanted, and fresh MeOH (50 mL) was added. The jar was returned to the oven that had been pre-heated to 60° C. This process was repeated for a total of three MeOH soaks. The bright green solid was filtered and transferred to a Schlenk flask. The material was activated under flowing N₂ at 180° C. for 4 h, and then by heating under high vacuum (<100 mTorr) at 180° C. for 24 h. During the activation procedure, the material changed in color from bright green to brown. The Schlenk flask was transferred into a N₂-filled glovebox, and the solid was transferred to a glass adsorption tube equipped with a Micromeritics TransSeal. The tube was removed from the glovebox and the material was activated for an additional 24 h under high vacuum (<10 pbar) at 180° C. Activated Ni₂(m-dobdc) was obtained as a yellow solid. Langmuir surface area determined from the 77 K N₂ adsorption isotherm: 1305±3 m²/g (Lit. 1592 m²/g). The PXRD pattern (CuKα radiation, k=1.5418 Å) of MeOH-solvated Ni₂(m-dobdc) is given in FIG. 9.

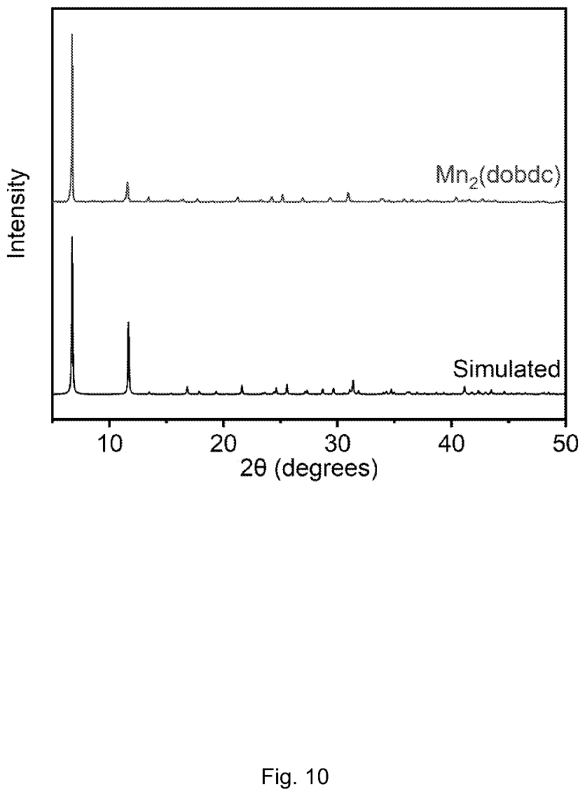

Mn2(dobdc). Prepared according to a modified previous literature procedure. A 75 mL screw-cap high pressure reaction vessel equipped with a stir bar was charged with MnCl₂•4H₂O (1.34 g, 6.77 mmol, 3.96 equiv.), 2,5-dihydroxyterephthalic acid (0.339 g, 1.71 mmol, 1.00 equiv.), fresh DMF (37.5 mL), and EtOH (2.5 mL). The reaction mixture was vigorously sparged with N₂ for 30 min. The reaction vessel was quickly capped, and the reaction mixture was sonicated until all of the solids were dissolved. The reaction vessel was transferred to a silicone oil bath, which was then heated to 135° C. The reaction mixture was allowed to stir slowly at 135° C. for 72 h, resulting in the precipitation of a yellow powder from solution. The reaction mixture was allowed to cool to room temperature, and the flask was transferred into a N₂-filled glovebox. The solution was removed from the yellow solid using a pipette, and fresh oxygen-free, dry DMF (40 mL) was added. The reaction vessel was capped, removed from the N₂-filled glovebox, placed in a silicone oil bath that had been pre-heated to 120° C., and allowed to stand at 120° C. for 24 h. At this time, the vessel was removed from the silicone oil bath, allowed to cool to room temperature, and transferred into a N₂-filled glovebox. The solution was removed from the yellow solid using a pipette, and oxygen-free, dry DMF (40 mL) was added. The reaction vessel was capped and removed from the N₂-filled glovebox. The reaction vessel was returned to a silicone oil bath that had been pre-heated to 120° C. This air-free soaking process was repeated for a total of six DMF soaks. After the final DMF soak, the solution was then pipetted off of the yellow solid in a N₂-filled glovebox, and N₂-sparged MeOH (40 mL) was added to the reaction vessel. The reaction vessel was capped, removed from the N₂-filled glovebox, placed in a silicone oil bath that had been pre-heated to 60° C., and allowed to stand at 60° C. for 24 h. At this time, the vessel was removed from the silicone oil bath, allowed to cool to room temperature, and transferred into a N₂-filled glovebox. The solution was then pipetted off of the yellow solid in a N₂-filled glovebox, and N₂-sparged MeOH (40 mL) was added to the reaction vessel. The reaction vessel was removed from the N₂-filled glovebox and placed in a silicone oil bath that had been pre-heated to 60° C. This air-free soaking process was repeated for a total of fifteen MeOH soaks to remove residual DMF (as judged by ATR-IR). The mixture was filtered in a N₂-filled glovebox, and the solid was quickly transferred to a Schlenk flask. The material was activated by heating under high vacuum (<100 mTorr) at 180° C. for 24 h. In a N₂-filled glovebox, the solid was transferred to a glass adsorption tube equipped with a Micromeritics TransSeal. The tube was removed from the glovebox and the material was activated for an additional 24 h under high vacuum (<10 pbar) at 180° C. Activated Mn₂(dobdc) was obtained as a yellow solid. Langmuir surface area determined from the 77 K N₂ adsorption isotherm: 1710±8 m²/g (Lit. 1797 m²/g) (4). The PXRD pattern (CuKα radiation, k=1.5418 Å) of MeOH-solvated Mn₂(dobdc) is given in FIG. 10. Note: Mn₂(dobdc) is air-sensitive and turns brown upon extended air exposure.

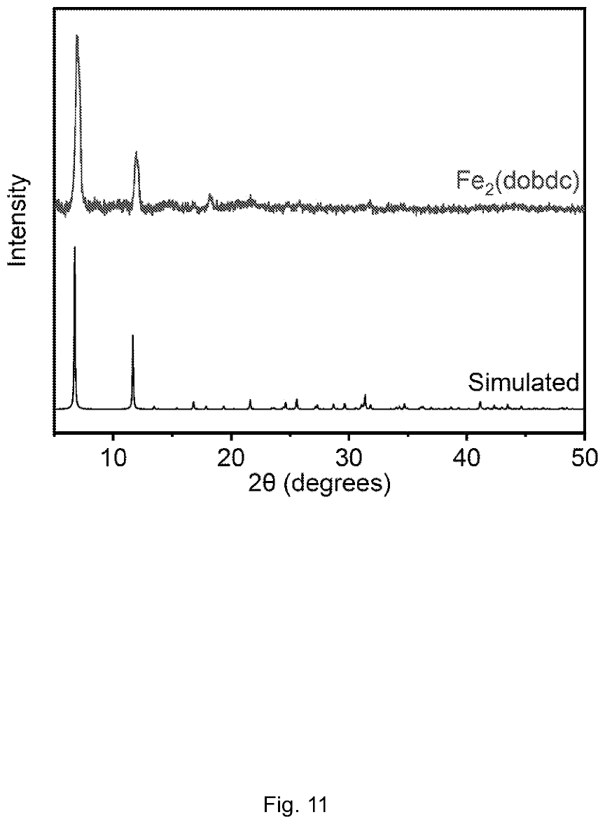

Fe₂(dobdc). Prepared according to a modified previous literature procedure. In a N₂-filled glovebox, a 350 mL screw-cap high pressure reaction vessel equipped with a stir bar was charged with FeCl₂ (0.550 g, 4.34 mmol, 2.42 equiv.), 2,5-dihydroxyterephthalic acid (0.355 g, 1.79 mmol, 1.00 equiv.), fresh oxygen-free, dry DMF (150 mL), and oxygen-free MeOH (18 mL). The reaction vessel was capped, removed from the N₂-filled glovebox, and placed in a silicone oil bath that had been pre-heated to 120° C. The reaction mixture was allowed to stir slowly at 120° C. for 18 h, resulting in the precipitation of an orange-red powder from solution. At this time, the vessel was removed from the silicone oil bath, allowed to cool to room temperature, and transferred into a N₂-filled glovebox. The solution was removed from the orange-red solid using a pipette, and fresh oxygen-free, dry DMF (150 mL) was added. The reaction vessel was capped, removed from the N₂-filled glovebox, placed in a silicone oil bath that had been pre-heated to 120° C. and allowed to stand at 120° C. for 24 h. At this time, the vessel was removed from the silicone oil bath, allowed to cool to room temperature, and transferred into a N₂-filled glovebox. The solution was removed from the orange-red solid using a pipette, and fresh oxygen-free, dry DMF (150 mL) was added. The reaction vessel was capped, removed from the N₂-filled glovebox, and returned to a silicone oil bath that had been pre-heated to 120° C. This soaking process was repeated for a total of four DMF soaks. After the final DMF soak, the solution was removed from the orange-red solid in a N₂-filled glovebox using a pipette, and oxygen-free MeOH (150 mL) was added. The reaction vessel was capped, removed from the glovebox, placed in a silicone oil bath heated to 60° C., and allowed to stand at 60° C. for 24 h. At this time, the vessel was removed from the silicone oil bath, allowed to cool to room temperature, and transferred into a N₂-filled glovebox. The solution was pipetted off of the now yellow solid in a N₂-filled glovebox, and fresh oxygen-free MeOH (150 mL) was added. The reaction vessel was capped, transferred out of the N₂-filled glovebox, and placed in a silicone oil bath that had been pre-heated to 60° C. This soaking process was repeated for a total of three MeOH soaks. The mixture was filtered in a N₂-filled glovebox, and the solid was quickly transferred to a Schlenk flask. The material was activated by heating under high vacuum (<100 mTorr) at 180° C. for 24 h. In a N₂-filled glovebox, the yellow solid was transferred to a glass adsorption tube equipped with a Micromeritics TransSeal. The tube was removed from the glovebox and the material was activated for an additional 24 h under high vacuum (<10 pbar) at 180° C. Activated Fe₂(dobdc) was obtained as a yellow solid. Langmuir surface area determined from the 77 K N₂ adsorption isotherm: 1481±20 m²/g (Lit. 1536 m²/g) (4). The PXRD pattern (CuKα radiation, k=1.5418 Å) of MeOH-solvated Fe₂(dobdc) is given in FIG. 11. Note: Fe₂(dobdc) is highly air-sensitive and turns black upon brief air exposure.

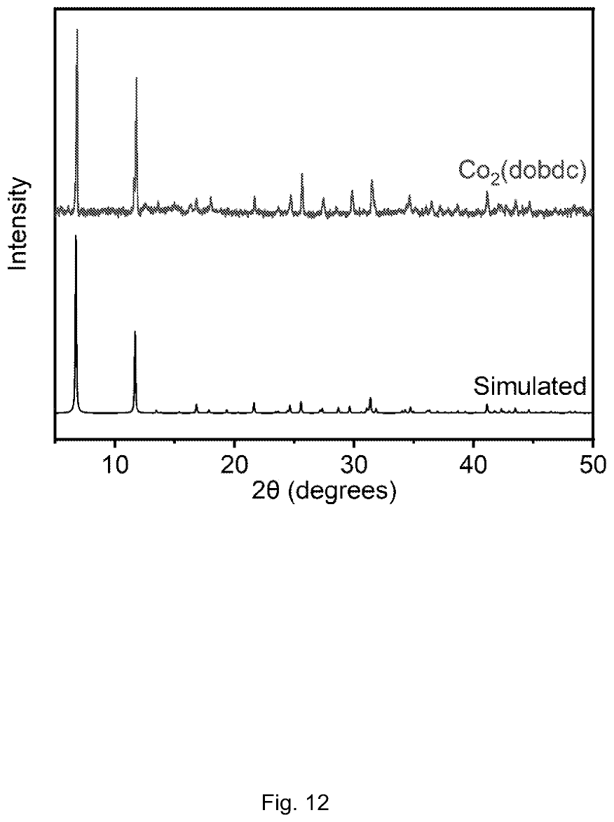

Co₂(dobdc). Prepared according to a previous literature procedure. A 250 mL Pyrex jar was charged with Co(NO₃)₂•6H₂O (1.94 g, 6.67 mmol, 3.34 equiv.), 2,5-dihydroxyterephthalic acid (0.396 g, 2.00 mmol, 1.00 equiv.), deionized water (25 mL), DMF (25 mL), and EtOH (25 mL). The mixture was sonicated until all of the solids were dissolved. The jar was placed in an oven that had been pre-heated to 100° C. and allowed to stand for 16 h, resulting in the precipitation of a dark purple solid from solution. The reaction mixture was allowed to cool to room temperature and filtered. The solid was quickly transferred to a 150 mL Pyrex jar filled with DMF (50 mL). The jar was transferred to an oven that had been pre-heated to 120° C. and allowed to stand at 120° C. for 24 h. At this time, the heterogeneous mixture was filtered. The solid was returned to the jar filled with fresh DMF (50 mL). The jar was returned to an oven that had been pre-heated to 120° C. This soaking process was repeated for a total of three DMF soaks. The mixture was then filtered and transferred to a 150 mL Pyrex jar filled with MeOH (50 mL). The jar was placed in an oven that had been pre-heated to 60° C. and allowed to stand at 60° C. for 24 h. At this time, the heterogeneous mixture was filtered. The solid was returned to the jar with fresh MeOH (50 mL) and the jar was returned to an oven that had been pre-heated to 60° C. This soaking process was repeated for a total of three MeOH soaks. The mixture was filtered again, and the solid was quickly transferred to a Schlenk flask under N₂. The material was activated under flowing N₂ at 180° C. for 24 h, and then by heating under high vacuum (<100 mTorr) at 180° C. for 24 h. The Schlenk flask was transferred into a N₂-filled glovebox, and the solid was transferred to a glass adsorption tube equipped with a Micromeritics TransSeal. The tube was removed from the glovebox and the material was activated for an additional 24 h under high vacuum (<10 pbar) at 180° C. Activated Co₂(dobdc) was obtained as dark purple crystals. Langmuir surface area determined from the 77 K N₂ adsorption isotherm: 1389±1 m²/g (Lit. 1438 m²/g) (4). The PXRD pattern (CuKα radiation, λ=1.5418 Å) of MeOH-solvated Co₂(dobdc) is given in FIG. 12.

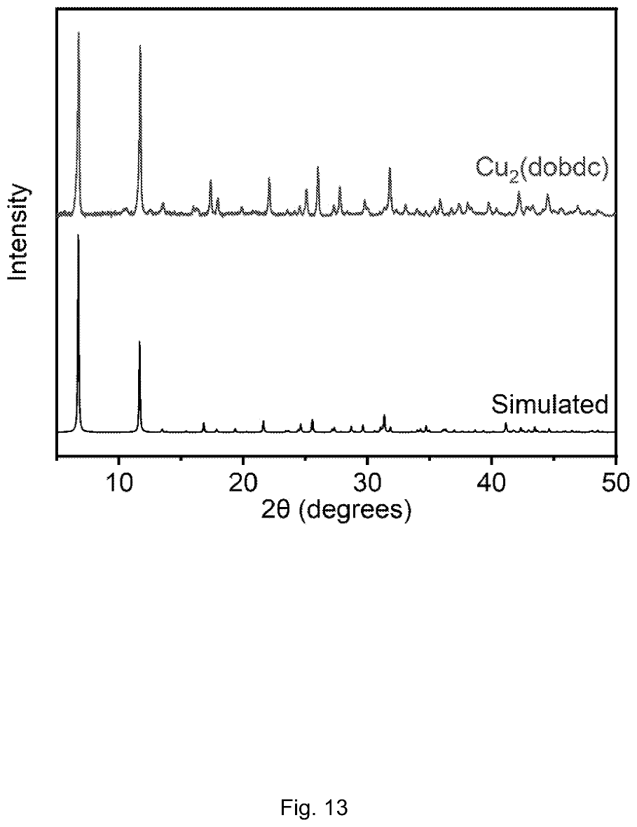

Cu₂(dobdc). Prepared according to a modified previous literature procedure. A 20 mL screw-cap vial was charged with Cu(NO₃)₂•2.5H₂O (87.2 mg, 0.375 mmol, 2.50 equiv.), 2,5-dihydroxyterephthalic acid (29.7 mg, 0.150 mmol, 1.00 equiv.), isopropanol (6 mL), and DMF (4 mL). The mixture was sparged with N₂ for 15 min, and the vial was capped. The mixture was sonicated until all of the solids were dissolved. The vial was placed in an aluminum heating block that had been pre-heated to 100° C. and allowed to stand at 100° C. for 45 min, resulting in the precipitation of a black powder from solution. The reaction mixture was allowed to cool to room temperature, and the solution was removed from the black solid using a pipette. The vial was filled with fresh DMF (15 mL). The vial was placed in an aluminum heating block that had been pre-heated to 70° C. and allowed to stand at 70° C. for 12 h. At this time, the heterogeneous mixture was allowed to cool to room temperature, the solution was removed from the black solid using a pipette, and fresh DMF (15 mL) was added to the vial. The vial was returned to an aluminum heating block that had been pre-heated to 70° C. This soaking process was repeated for a total of six DMF soaks. After the final DMF soak, the solution was removed from the black solid using a pipette and fresh MeOH (15 mL) was added. The vial was placed in an aluminum heating block that had been pre-heated to 60° C. and allowed to stand at 60° C. for 12 h. At this time, the heterogeneous mixture was allowed to cool to room temperature. The solution was removed from the black solid using a pipette and fresh MeOH (15 mL) was added to the vial. The vial was returned to an aluminum heating block that had been pre-heated to 60° C. This soaking process was repeated for a total of six MeOH soaks. The mixture was filtered again, and the solid was quickly transferred to a Schlenk flask under N₂. The material was activated under flowing N₂ at 180° C. for 1 h, and then by heating under high vacuum (<100 mTorr) at 180° C. for 24 h. The Schlenk flask was transferred into a N₂-filled glovebox, and the solid was transferred to a glass adsorption tube equipped with a Micromeritics TransSeal. The tube was removed from the glovebox and the material was activated for an additional 24 h under high vacuum (<10 pbar) at 180° C. Activated Cu₂(dobdc) was obtained as a black solid. Langmuir surface area determined from the 77 K N₂ adsorption isotherm: 1847 m²/g±46 m²/g (Lit. 1515 m²/g) (4). The PXRD pattern (CuKα radiation, k=1.5418 Å) of MeOH-solvated Cu₂(dobdc) is given in FIG. 13.

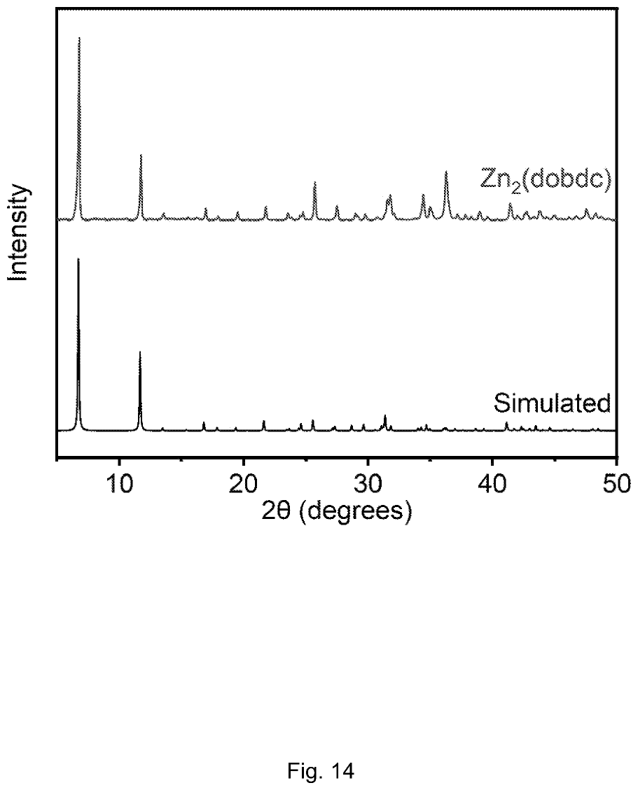

Zn₂(dobdc). Prepared according to a previous literature procedure. A 350 mL screw-cap high pressure reaction vessel equipped with a stir bar was charged with Zn(NO₃)₂•6H₂O (2.23 g, 7.50 mmol, 3.00 equiv.), 2,5-dihydroxyterephthalic acid (0.495 g, 2.50 mmol, 1.00 equiv.), fresh DMF (125 mL), and EtOH (125 mL). The mixture was sonicated until all of the solids were dissolved. The reaction mixture was vigorously sparged with N₂ for 1 h. The reaction vessel was capped, and the reaction mixture was transferred to a silicone oil bath and heated to 120° C. The reaction mixture was allowed to stir slowly at 120° C. for 14 h, resulting in the precipitation of a yellow powder from solution. The reaction mixture was allowed to cool to room temperature and filtered. The solid was quickly transferred to a 500 mL Pyrex jar filled with DMF (250 mL). The jar was placed in an oven that had been pre-heated to 120° C. and allowed to stand at 120° C. for 24 h. At this time, the heterogeneous mixture was filtered. The solid was returned to the jar with fresh DMF (250 mL) and returned to an oven that had been pre-heated to 120° C. This soaking process was repeated for a total of three DMF soaks. The mixture was then filtered and transferred to a 500 mL Pyrex jar filled with MeOH (250 mL). The jar was placed in an oven that had been pre-heated to 60° C. and allowed to stand at 60° C. for 24 h. At this time, the heterogeneous mixture was filtered. The solid was returned to the jar with fresh MeOH (250 mL) and returned to an oven that had been pre-heated to 60° C. This soaking process was repeated for a total of three MeOH soaks. The mixture was filtered again, and the solid was quickly transferred to a Schlenk flask under N₂. The material was activated under flowing N₂ at 180° C. for 24 h, and then by heating under high vacuum (<100 mTorr) at 180° C. for 24 h. The Schlenk flask was transferred into a N₂-filled glovebox, and the solid was transferred to a glass adsorption tube equipped with a Micromeritics TransSeal. The tube was removed from the glovebox and the material was activated for an additional 24 h under high vacuum (<10 pbar) at 180° C. Activated Zn₂(dobdc) was obtained as a yellow solid. Langmuir surface area determined from the 77 K N₂ adsorption isotherm: 1368 m²/g±3 m²/g (Lit. 1277 m²/g). The PXRD pattern (CuKα radiation, k=1.5418 Å) of MeOH-solvated Zn₂(dobdc) is given in FIG. 14.

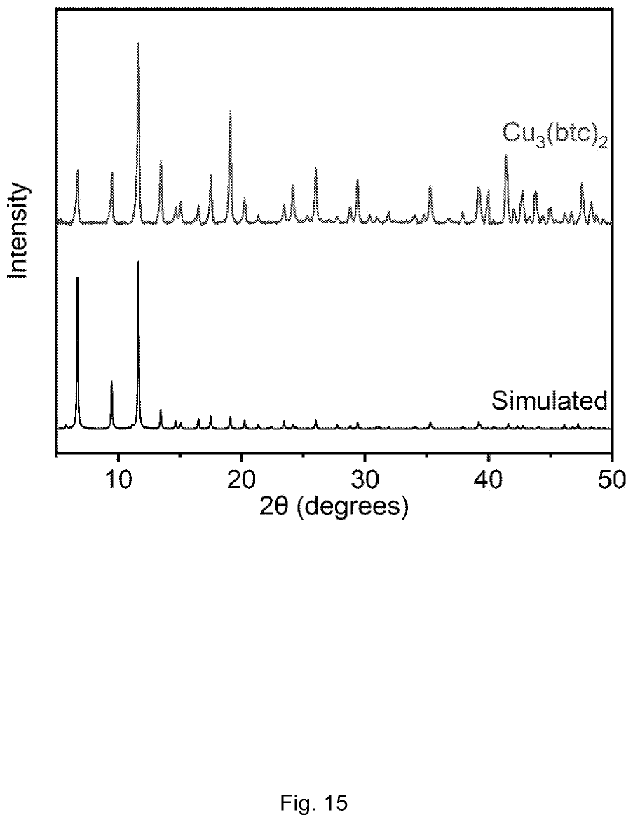

Cu₃(btc)₂. Prepared according to a modified previous literature procedure. A 500 mL round-bottom flask equipped with a stir bar was charged with Cu(Oac)₂•H₂O (8.58 g, 43.0 mmol, 1.81 equiv.), 1,3,5-benzenetricarboxylic acid (5.00 g, 23.8 mmol, 1.00 equiv.), H₂O (80 mL), fresh DMF (80 mL), and EtOH (80 mL). The flask was sealed with a glass stopper and transferred to a silicone oil bath. The reaction mixture was allowed to stir slowly at 80° C. for 24 h, resulting in the precipitation of a bright blue solid from solution. The reaction mixture was allowed to cool to room temperature and filtered, rinsing with H₂O (200 mL) and EtOH (100 mL). The solid was quickly transferred to a 500 mL Pyrex jar filled with EtOH (200 mL) and allowed to stand at room temperature for 24 h. At this time, the heterogeneous mixture was filtered, and the solid was returned to the jar with fresh EtOH (200 mL). This soaking process was repeated for a total of two EtOH soaks. The mixture was then filtered, and the solid was transferred to a 500 mL Pyrex jar filled with MeOH (200 mL) and allowed to stand at room temperature for 24 h. At this time, the heterogeneous mixture was filtered, and the solid was returned to the jar with fresh MeOH (200 mL). This soaking process was repeated for a total of two MeOH soaks. The mixture was filtered again, and the solid was quickly transferred to a Schlenk flask under N₂. The material was activated under flowing N₂ at 150° C. for 8 h, and then by heating under high vacuum (<100 mTorr) at 150° C. for 24 h. The Schlenk flask was transferred into a N₂-filled glovebox, and the solid was transferred to a glass adsorption tube equipped with a Micromeritics TransSeal. The tube was removed from the glovebox and the material was activated for an additional 24 h under high vacuum (<10 pbar) at 150° C. Activated Cu₃(btc)₂ was obtained as a dark blue solid. Langmuir surface area determined from the 77 K N₂ adsorption isotherm: 2078±2 m²/g (Lit. 2203 m²/g). The PXRD pattern (CuKα radiation, k=1.5418 Å) of MeOH-solvated Cu₃(btc)₂ is given in FIG. 15.

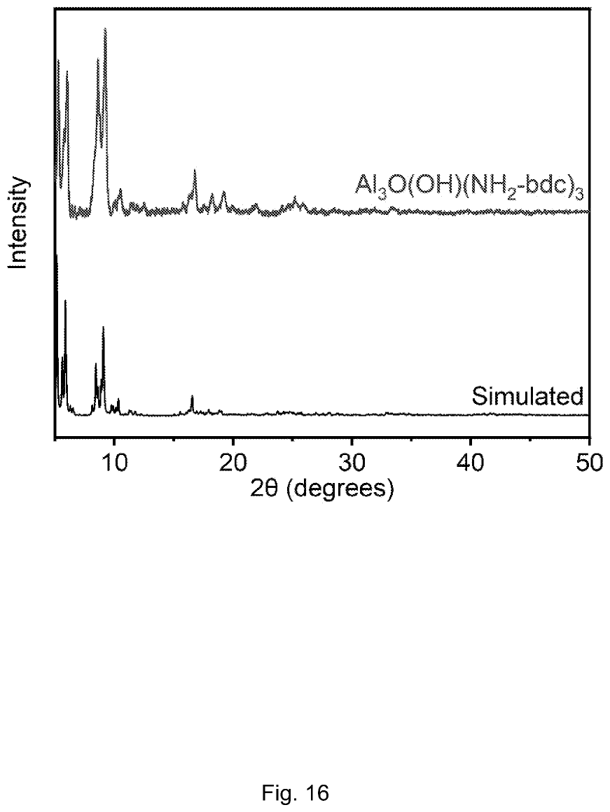

Al₃O(OH)(NH₂-bdc)₃. Prepared according to a modified previous literature procedure. A 15 mL screw-cap high pressure reaction vessel equipped with a stir bar was charged with AlCl₃.6H₂O (88.1 mg, 0.365 mmol, 1.00 equiv.), 2-aminoterephthalic acid (93.5 mg, 0.516 mmol, 1.41 equiv.), and fresh DMF (5 mL). The mixture was sonicated until all of the solids were dissolved. The reaction vessel was capped and transferred to a silicone oil bath. The reaction mixture was allowed to stir slowly at 130° C. for 72 h, resulting in the precipitation of a yellow powder from solution. The reaction mixture was allowed to cool to room temperature and filtered. The solid was quickly transferred to a 10 mL screw-cap tube filled with MeOH (5 mL). The tube was placed in an aluminum heating block that had been pre-heated to 60° C. and allowed to stand at 60° C. for 24 h. At this time, the tube was removed from the block and allowed to cool to room temperature. The solution was removed from the yellow solid using a pipette, and fresh MeOH (5 mL) was added. The tube was returned to an aluminum heating block that had been pre-heated to 60° C. This soaking process was repeated for a total of three MeOH soaks. The mixture was filtered again, and the solid was quickly transferred to a Schlenk flask under N₂. The material was activated under flowing N₂ at 100° C. for 1 h, and then by heating under high vacuum (<100 mTorr) at 100° C. for 24 h. The Schlenk flask was transferred into a N₂-filled glovebox, and the solid was transferred to a glass adsorption tube equipped with a Micromeritics TransSeal. The tube was removed from the glovebox, and the material was activated for an additional 24 h under high vacuum (<10 pbar) at 180° C. Activated Al₃O(OH)(NH₂-bdc)₃ was obtained as a yellow solid. Langmuir surface area determined from the 77 K N₂ adsorption isotherm: 2040 m²/g±8 m²/g (Lit. 2890 m²/g). The PXRD pattern (CuKα radiation, k=1.5418 Å) of MeOH-solvated Al₃O(OH)(NH₂-bdc)₃ is given in FIG. 16.

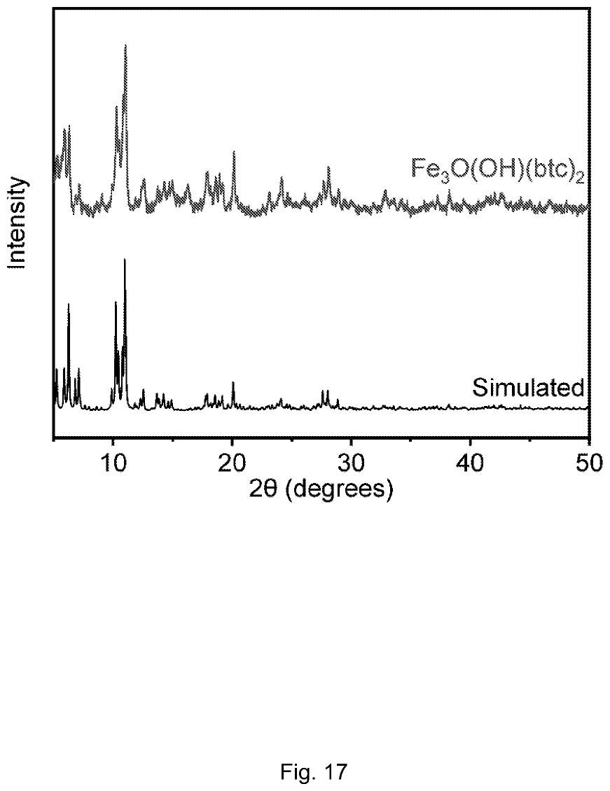

Fe₃O(OH)(btc)₂. Prepared according to a modified literature procedure. A 10 mL screw-cap tube was charged with Fe(NO₃)₃•9H₂O (80.8 mg, 0.200 mmol, 1.50 equiv.), 1,3,5-benzenetricarboxylic acid (27.9 mg, 0.133 mmol, 1.00 equiv.), trifluoroacetic acid (300. μL, 3.92 mmol, 29.5 equiv.), and deionized water (2 mL). The mixture was sonicated until all of the solids were dissolved. The tube was capped, and the reaction mixture was heated at 130° C. for 24 h, resulting in the precipitation of a powder from solution. The reaction mixture was allowed to cool to room temperature and filtered. The solid was quickly transferred to a 10 mL screw-cap tube with fresh DMF (2 mL). The tube was capped, placed in an aluminum heating block that had been pre-heated to 120° C. and allowed to stand at 120° C. for 24 h. At this time, the heterogeneous mixture was allowed to cool to room temperature, the solution was removed from the solid using a pipette, and fresh DMF (2 mL) was added. The tube was capped and returned to an aluminum heating block that had been pre-heated to 120° C. This soaking process was repeated for a total of three DMF soaks. The mixture was filtered again, and the solid was quickly transferred to a 10 mL screw-cap tube with deionized water (2 mL). The tube was capped, placed in an aluminum heating block that had been pre-heated to 95° C., and allowed to stand at 95° C. for 24 h. At this time, the heterogeneous mixture was allowed to cool to room temperature, the solution was removed from the solid using a pipette, and fresh deionized water (2 mL) was added. The tube was returned to an aluminum heating block that had been pre-heated to 95° C. This soaking process was repeated for a total of three deionized water soaks. The mixture was filtered again, and the solid was quickly transferred to a 10 mL screw-cap tube filled with EtOH (2 mL). The tube was capped, placed in an aluminum heating block that had been pre-heated to 65° C., and allowed to stand at 65° C. for 24 h. At this time, the heterogeneous mixture was allowed to cool to room temperature, the solution was removed from the solid using a pipette, and fresh EtOH (2 mL) was added to the tube. The tube was returned to an aluminum heating block that had been pre-heated to 65° C. This soaking process was repeated for a total of three EtOH soaks. The mixture was filtered again, and the solid was quickly transferred to a Schlenk flask. The material was activated by heating under high vacuum (<100 mTorr) at 180° C. for 24 h. The Schlenk flask was transferred into a N₂-filled glovebox, and the solid was transferred to a glass adsorption tube equipped with a Micromeritics TransSeal. The tube was removed from the glovebox and the material was activated for an additional 24 h under high vacuum (<10 pbar) at 180° C. Activated Fe₃O(OH)(btc)₂ was obtained as a light orange solid. Langmuir surface area determined from the 77 K N₂ adsorption isotherm: 2387±15 m²/g (Lit. 2007 m²/g). The PXRD pattern (CuKα radiation, k=1.5418 Å) of MeOH-solvated Fe₃O (OH)(btc)₂ is given in FIG. 17.

Zeolite Y, Sodium. The Zeolite Y, Sodium used in this work was purchased from Alfa Aesar and used without additional purification. The material was transferred to a Schlenk flask and activated by heating under high vacuum (<100 mTorr) at 180° C. for 24 h. The Schlenk flask was transferred into a N₂-filled glovebox, and the solid was transferred to a glass adsorption tube equipped with a Micromeritics TransSeal. The tube was removed from the glovebox and the material was activated for an additional 24 h under high vacuum (<10 pbar) at 180° C. Langmuir surface area determined from the 77 K N₂ adsorption isotherm: 905±1 m²/g (Alfa Aesar: 900 m²/g).

Summary of Storage Capacities and Binding Enthalpies for Fluorinated Gases in Different Materials.

TABLE 1
Storage capacity (mmol/g) for VDF in different materials (1
bar VDF; 30° C., 40° C., 50° C.).

| Material | 30° C. | 40° C. | 50° C. |
|---|---|---|---|
| Mg₂(dobdc) | 7.95 | 7.35 | 6.80 |
| Mn₂(dobdc) | 6.52 | 5.91 | 3.80 |
| Fe₂(dobdc) | 6.79 | 6.16 | 5.60 |
| Co₂(dobdc) | 6.59 | 6.11 | 5.58 |
| Ni₂(dobdc) | 5.75 | 5.34 | 4.94 |
| Cu₂(dobdc) | 6.36 | 5.42 | 4.78 |
| Zn₂(dobdc) | 4.86 | 4.41 | 3.99 |
| Mg₂(m-dobdc) | 5.96 | 5.52 | 5.06 |
| Ni₂(m-dobdc) | 5.54 | 5.16 | 4.77 |
| Cu₃(btc)₂ | 7.64 | 6.78 | 5.81 |
| Fe₃O(OH)(btc)₂ | 3.05 | 2.60 | 2.22 |
| Al₃O(OH)(NH₂-bdc)₃ | 2.07 | 1.99 | 1.46 |
| Zeolite Y | 3.74 | 3.54 | 3.34 |

TABLE 2
Storage capacity (mmol/g) for TFP in different materials (1 bar TFP;
30° C., 40° C., 50° C., 60° C.).

| Material | 30° C. | 40° C. | 50° C. | 60° C. |
|---|---|---|---|---|
| Mg₂(dobdc) | 7.84 | 7.45 | 6.96 | — |
| Ni₂(dobdc) | 5.06 | 4.80 | 4.56 | 4.34 |
| Ni₂(m-dobdc) | 4.50 | 4.32 | 4.13 | 3.95 |

TABLE 3
Storage capacity (mmol/g) for HFP in different materials (1
bar HFP; 30° C., 40° C., 50° C.).

| Material | 30° C. | 40° C. | 50° C. |
|---|---|---|---|
| Mg₂(dobdc) | 5.78 | 5.52 | 5.27 |
| Ni₂(dobdc) | 3.02 | 2.87 | 2.75 |
| Ni₂(m-dobdc) | 3.60 | 3.45 | 3.30 |

TABLE 4
Storage capacity (mmol/g) for TFMI in different materials (1 bar TFMI;
30° C., 40° C., 50° C., 60° C.).

| Material | 30° C. | 40° C. | 50° C. | 60° C. |
|---|---|---|---|---|
| Mg₂(dobdc) | 8.90 | 8.38 | 8.01 | 7.57 |
| Ni₂(dobdc) | 5.43 | 5.39 | 5.17 | — |
| Ni₂(m-dobdc) | 5.13 | 4.94 | 4.72 | 4.53 |

TABLE 5
Binding enthalpy (−ΔH_{ads}, kJ/mol) for VDF at
a loading of 1 mmol/g in different materials.

| Material | −ΔHads |
|---|---|
| Mg₂(dobdc) | 38.0 ± 2.74 |
| Mn₂(dobdc) | 34.3 ± 0.64 |
| Fe₂(dobdc) | 34.6 ± 0.61 |
| Co₂(dobdc) | 36.1 ± 0.57 |
| Ni₂(dobdc) | 40.4 ± 1.73 |
| Cu₂(dobdc) | 30.7 ± 2.09 |
| Zn₂(dobdc) | 31.7 ± 1.02 |
| Mg₂(m-dobdc) | 39.2 ± 0.06 |
| Ni₂(m-dobdc) | 41.7 ± 0.17 |
| Cu₃(btc)₂ | 32.8 ± 0.09 |
| Fe₃O(OH)(btc)₂ | 30.8 ± 1.02 |
| Al₃O(OH)(NH₂-bdc)₃ | 22.2 ± 3.00 |
| Zeolite Y | 40.4 ± 0.26 |

TABLE 6
Binding enthalpy (−ΔH_{ads}, kJ/mol) for TFP at
a loading of 1 mmol/g in different materials.

| Material | −ΔHads |
|---|---|
| Mg₂(dobdc) | 50.1 ± 0.67 |
| Ni₂(dobdc) | 43.7 ± 0.57 |
| Ni₂(m-dobdc) | 37.4 ± 3.62 |

TABLE 7
Binding enthalpy (−ΔH_{ads}, kJ/mol) for HFP at
a loading of 1 mmol/g in different materials.

| Material | −ΔHads |
|---|---|
| Mg₂(dobdc) | 46.7 ± 0.57 |
| Ni₂(dobdc) | 42.8 ± 0.50 |
| Ni₂(m-dobdc) | 47.2 ± 1.94 |

TABLE 8
Binding enthalpy (−ΔH_{ads}, kJ/mol) for TFMI at
a loading of 1 mmol/g in different materials.

| Material | −ΔHads |
|---|---|
| Mg₂(dobdc) | 26.1 ± 3.73 |
| Ni₂(dobdc) | 10.2 ± 0.87 |
| Ni₂(m-dobdc) | 11.1 ± 0.74 |

Experimental Procedure for MAS ¹⁹F SSNMR Spectroscopy on VDF-Mg₂(dobdc). An oven-dried 10 mL round-bottom flask and a Schlenk adapter were cycled into a N₂-filled glovebox. In the glovebox, activated Mg₂(dobdc) was loaded into the round-bottom flask. The flask was sealed with the Schlenk adapter and removed from the glovebox. The flask containing Mg₂(dobdc) was connected to a Schlenk line and the gas cylinder through a three-way valve. The three-way valve was used to pull vacuum on the regulator of the gas cylinder and to reactivate the MOF under high vacuum (<100 mTorr) while ramping the temperature to 300° C. (1° C./min) using a sand bath. The flask allowed to stand at 300° C. under high vacuum (<100 mTorr) for 12 h. After activation, the flask was allowed to cool to room temperature under vacuum. Once cool, the joints of the flask containing the Mg₂(dobdc) were secured with copper wire, and the three-way valve was closed to the Schlenk line. The freshly activated sample of Mg₂(dobdc) was immediately dosed with approximately 1 bar of VDF at approximately 0° C. (in an ice-water bath) for 40 min. After 40 min, the flask was closed with the Schlenk adapter under approximately 1 bar of VDF, and the freshly prepared VDF-Mg₂(dobdc) was brought into a N₂-filled glovebox with the rotor and packing tools. The rotor was quickly packed and transferred to a 20 mL scintillation vial, which was then wrapped in Teflon tape before being brought out of the glovebox and transported to the NMR spectrometer for immediate analysis. MAS ¹⁹F SSNMR (470 MHz) δ −87.34 ppm.

Computational and Structural Details. Density Functional Theory Calculated Structures. To compute binding energies of fluorinated gas molecules in Mg₂(dobdc), first-principles density functional theory (DFT) calculations were performed using optimized norm-conserving Vanderbilt pseudopotentials (ONCV) (21) with the Quantum ESPRESSO plane wave DFT code (22). To include the effect of the van der Waals (vdW) dispersive interactions on binding energies, structural relaxations were performed with revised vdW-DF2 functional (23) as implemented in the Quantum ESPRESSO. For all calculations, (i) a Γ-point sampling of the Brillouin zone and (ii) a 220 Ry plane-wave cutoff energy were used. Seven valence electrons for I (5s²5p⁵), ten for Mg (2s²2p⁶3s²), seven for F (2s²2p⁵), six for 0 (2s²2p⁴), five for N (2s²2p³), four for C (2s²2p²), and one for H(1s^{l}) were explicitly treated. Using the above input parameters, both the unit cell volume and internal coordinates of Mg₂(dobdc) were fully relaxed. All structural relaxations were performed with a Gaussian smearing of 0.003 Ry. The ions were relaxed until both the force and energy are less than 5×10⁻⁴ Ry/Bohr and 1×10⁻⁶ Ry. To compute the binding energies of organic molecules, the following energies were optimized while fixing the optimized lattice parameters: Mg₂(dobdc) prior to fluorinated gas adsorption (E_{Mg}-M_{OF}), the interaction of fluorinated gas molecules in the gas phase (Eₘₒₗ) within a 15 Å×15 Å×15 Å cubic supercell using dipole corrections (25), and the interaction of Mg₂(dobdc) with adsorbed fluorinated gas molecules (E_{mol-Mg-MOF}). The binding energies (E_{B}) were obtained using Equation 3:

$${E}_{B} = {E}_{mol - Mg - MOF} - ( {E}_{Mg - MOF} + {E}_{mol} ) \qquad Equation 3$$

To compare these calculations with experimentally-determined heats of adsorption, zero-point energy (ZPE) and thermal energy (TE) corrections were computed, following previous DFT studies. For these calculations, the vibrational frequencies were calculated using density functional perturbation theory (DFPT) as implemented in the Quantum ESPRESSO. All of the TE corrections were computed at 298 K. In the cases of VDF, TFP, and HFP, two distinct binding modes, either alkene-bound or fluorine-bound, were identified. These binding modes were similar in energy in all three cases. In the case of TFMI, two distinct binding modes, iodine-bound and fluorine-bound, were identified. The F-bound structure displayed a closer match the experimental −ΔH_{ads} value.

TABLE 9
Computed binding energies (E_{B}), zero-point energy (ZPE) and
thermal energy (TE) corrections, and binding enthalpies (H_{B}) (in
kJ/mol) of fluorinated gas molecules in Mg₂(dobdc).

| Gas | Binding Mode | E_{B} | ZPE | TE | H_{B} | Experimental H_{B} |
|---|---|---|---|---|---|---|
| VDF | alkene-Mg | −47.8 | 3.9 | 1.2 | −42.7 | −37.7 |
| VDF | F—Mg | −41.8 | 2.4 | 1.9 | −37.5 | −37.7 |
| TFP | alkene-Mg | −67.1 | 1.8 | 2.3 | −63.1 | −51.1 |
| TFP | F—Mg | −54.6 | 0.1 | 3.2 | −51.3 | −51.1 |
| HFP | F—Mg | −52.5 | 2.4 | 2.5 | −47.6 | −47.3 |
| HFP | alkene-Mg | −49.5 | 1.7 | 2.8 | −44.9 | −47.3 |
| TFMI | I—Mg | −56.8 | 0.6 | 3.3 | −53.0 | −26.3 |
| TFMI | F—Mg | −29.7 | −0.7 | 4.1 | −26.3 | −26.3 |

Powder X-ray Diffraction Structure of VDF-Mg₂(dobdc). High-resolution PXRD patterns of VDF-Mg₂(dobdc) samples were collected with synchrotron X-ray radiation at beamline 11-BM at the Advanced Photon Source at Argonne National Laboratory. The average wavelength of the X-rays was set to 0.458177 Å. Data was collected using a multi-analyzer detection setup which is composed of 12 independent Si (111) crystal analyzers and LaCl₃ scintillation detectors. Prior to collecting diffraction data, the Mg₂(dobdc) was gently ground into a fine powder using a mortar and pestle and packed into borosilicate glass capillaries 1 mm in diameter (Hilgenberg glass No. 50). The MOF sample activated under high vacuum (<100 mTorr) at 180° C. overnight. The sample was allowed to cool under vacuum before it was dosed with −100 mbar of VDF at room temperature using a custom-built, capillary-dosing manifold equipped with a gas dosing. Finally, the capillary was flame-sealed under 100 mbar of VDF. Diffraction patterns were collected at 295 K. The PXRD pattern was analyzed with the software TOPAS-Academic V6. Precise unit cell parameters were obtained by Pawley fitting. The crystal structure was solved by the real-space global optimization simulated annealing (SA) method. During the SA runs, the crystal structure of Mg₂(dobdc) was kept fixed, whereas the position and torsion angles of two VDF molecules (defined in Z-matrix notation) were freely varied. Once the SA optimization converged to a global minimum, the structure was subjected to Rietveld refinement. During the refinement, the following parameters were set free: unit cell and peak profile parameters, background coefficients (described as a Chebyshev polynomial), site occupancies, positions of selected atoms (defined as a single variable parameter for each VDF molecule), bond distances within the rigid bodies, and thermal displacement parameters (defined as a single variable for each atomic type, except for hydrogen atoms that were calculated as the thermal displacement parameter of the bonded atom multiplied by a factor of 1.5).

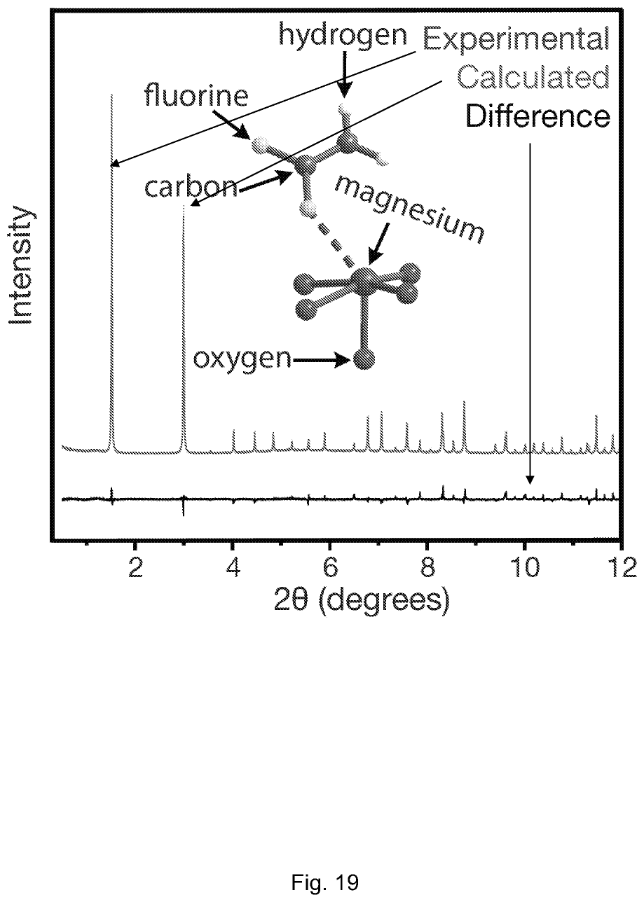

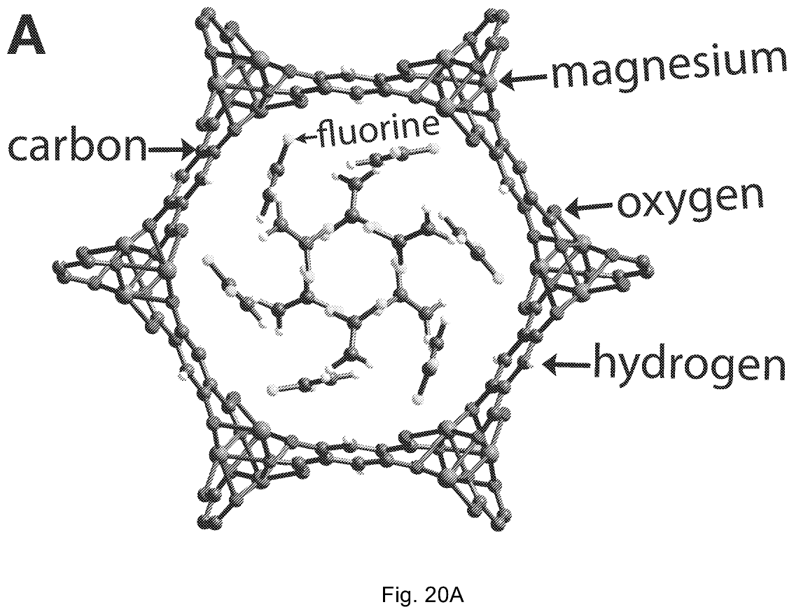

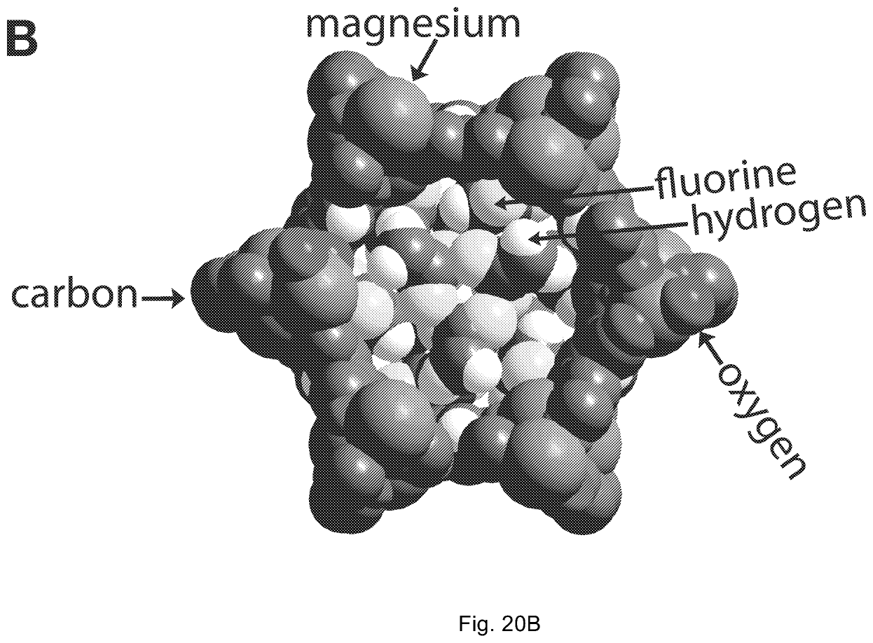

The Rietveld refinement of the obtained pattern corroborated a structural model in which VDF binds to the open Mg²⁺ site through a F . . . Mg interaction. The corresponding weighted residual factor (R_{wp}) for this structural model is 11.9%. The slight residual confounding can be attributed to additional features that cannot be modeled, such as trace H₂O interacting with the open metal sites or a small fraction of VDF interacting with the MOF through the alkene. The Rietveld plot is given in FIG. 19 and the structural model is given in FIG. 20 and in the inset of FIG. 19.

### Synthesis of Catalysts and Starting Materials.

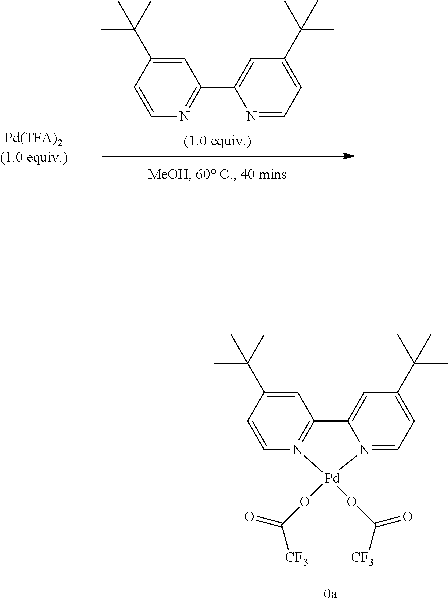

Pd(TFA)₂(dtbbpy) (0a). A 20 mL scintillation vial was charged with a stir bar, palladium(II) trifluoroacetate (306 mg, 0.920 mmol, 1.00 equiv.), 4,4′-di-tert-butyl-2,2′-bipyridine (247 mg, 0.920 mmol, 1.00 equiv.), and MeOH (4 mL). The heterogeneous reaction mixture was heated to 60° C. and stirred for 40 min open to air. At this time, the reaction mixture was vacuum filtered, and the resulting solid was rinsed with hot MeOH (˜60° C.) three times to afford Pd(TFA)₂(dtbbpy) (318.9 mg, 58%) as a yellow solid. ¹H NMR (500 MHz, CD₂Cl₂): δ 8.01-7.96 (m, 4H), 7.56 (dt, J=6.2, 1.7 Hz, 2H), 1.43 (s, 18H) ppm. ¹³C NMR (126 MHz, CD₂Cl₂): δ 167.23, 162.39 (q, J=36.8 Hz), 156.25, 149.38, 124.87, 120.20, 115.19 (q, J=290.2 Hz), 53.84 (dt, J=54.5, 27.2 Hz), 36.35, 30.28 ppm. 19F NMR (376 MHz, CDCl₃): δ −74.23 ppm. IR (neat, cm⁻¹): 2973 (w), 2878 (w), 1697 (w), 1617 (w), 1578 (w), 1547 (w), 1485 (w), 1466 (w), 1403 (w), 1370 (w), 1311 (w), 1252 (w), 1182 (m), 1131 (w), 1048 (w), 1037 (w), 902 (w), 893 (w), 844 (w), 788 (w), 728 (m), 622 (w), 600 (w), 558 (w), 523 (w).

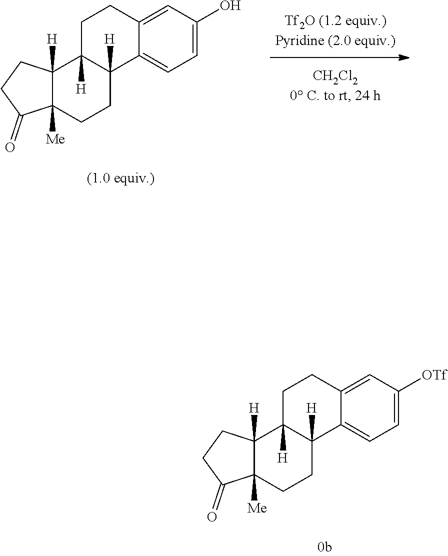

(8R,9S,13S,14S)-13-methyl-17-oxo-7,8,9,11,12,13,14,15,16,17-decahydro-6H-cyclopenta[a]phenanthrene-3-yl trifluoromethanesulfonate (0b). Adapted from a previous literature procedure. A 25 mL three-neck round bottom flask was flame-dried under high vacuum and allowed to cool under vacuum. Under flowing N₂, the three-neck was charged with a stir bar and estrone (1.00 g, 3.70 mmol, 1.00 equiv.). The flask was evacuated and back-filled with N₂. This process was repeated a total of three times. Under flowing N₂, anhydrous pyridine (600. μL, 7.45 mmol, 2.01 equiv.) and anhydrous CH₂Cl₂ (20 mL) were added to the flask. The flask was cooled to 0° C. using an ice-water bath. Trifluoromethanesulfonic anhydride (760. μL, 4.52 mmol, 1.22 equiv.) was added dropwise. The reaction mixture was allowed to warm to room temperature and was stirred at room temperature for 24 h. At this time, the reaction mixture was diluted with water, and the phases were separated. The aqueous phase was extracted with CH₂Cl₂ (3×50 mL). The combined organic layers were concentrated under reduced pressure, and the crude product was purified by flash chromatography using a Biotage Isolera instrument (SiO₂, gradient of 0%→20% EtOAc in hexanes) to afford (8R,9S,13S,14S)-13-methyl-17-oxo-7,8,9,11,12,13,14,15,16,17-decahydro-6H-cyclopenta[a]-55-henanthrene-3-yl trifluoromethanesulfonate (887.2 mg, 60%) as a white solid. ¹H NMR (500 MHz, CDCl₃): δ 7.34 (d, J=8.7 Hz, 1H), 7.03 (dd, J=8.7, 2.7 Hz, 1H), 6.99 (d, J=2.8 Hz, 1H), 2.94 (dd, J=8.9, 4.2 Hz, 2H), 2.52 (dd, J=19.0, 8.8 Hz, 1H), 2.45-2.36 (m, 1H), 2.30 (td, J=10.9, 4.0 Hz, 1H), 2.16 (dt, J=18.6, 8.9 Hz, 1H), 2.05 (ddt, J=15.9, 6.9, 4.9 Hz, 2H), 2.01-1.93 (m, 1H), 1.73-1.56 (m, 2H), 1.55-1.42 (m, 4H), 0.92 (s, 3H) ppm. ¹³C NMR (126 MHz, CDCl₃): δ): δ220.55, 147.72, 140.41, 139.43, 127.33, 121.38, 118.88 (q, J=321.7 Hz), 118.44, 50.51, 47.99, 44.23, 37.88, 35.94, 31.61, 29.52, 26.22, 25.82, 21.70, 13.93 ppm. 19F NMR (470 MHz, CDCl₃): δ −72.96 ppm. The NMR spectra are in agreement with those reported in the literature.

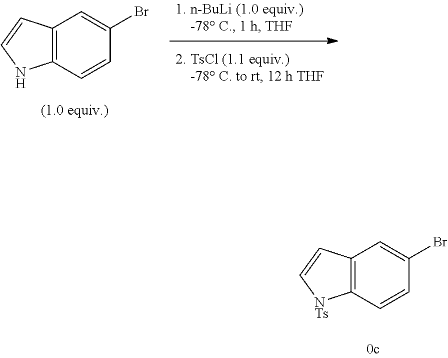 [SMILES: Brc1ccc2[nH]ccc2c1 ; Cc1ccc(S(=O)(=O)n2ccc3cc(Br)ccc32)cc1]

5-bromo-1-tosyl-1H-indole (0c). Adapted from a previous literature procedure. A 25 mL two-neck round-bottom flask was charged with 5-bromo-1H-indole (1.51 g, 7.70 mmol, 1.00 equiv.) and a stir bar. The flask was evacuated and back-filled with N₂. This process was repeated a total of three times. Under flowing N₂, anhydrous THE (1.3 mL) was added to the flask. The flask was cooled to −78° C. using a dry ice/acetone bath. Next, n-butyllithium (2.5 M in hexanes, 3.1 mL, 7.8 mmol, 1.0 equiv.) was added dropwise. The reaction mixture was warmed to 0° C. and stirred for 1 h. The reaction was again cooled to −78° C. using a dry ice/acetone bath. The stopper was removed before quickly adding p-toluenesulfonyl chloride (1.60 g, 8.39 mmol, 1.09 equiv.) under positive N₂ flow. The reaction was allowed to warm to room temperature and was stirred for 12 h. The mixture was diluted with 5% aq. NaHCO₃ (10 mL), and the aqueous phase was extracted with CH₂Cl₂ (3×50 mL). The combined organic extracts were washed with water (50 mL) and brine (50 mL). The organic layer was concentrated under reduced pressure to afford a light-brown solid. The crude solid was purified by recrystallization from Et₂O:CH₂Cl₂ (6:1) to afford 5-bromo-1-tosyl-1H-indole (207 mg, 8%) as an off-white solid. ¹H NMR (500 MHz, CDCl₃): δ 7.86 (d, J=8.8 Hz, 1H), 7.78-7.71 (m, 2H), 7.65 (d, J=1.9 Hz, 1H), 7.56 (d, J=3.6 Hz, 1H), 7.39 (dd, J=8.8, 1.9 Hz, 1H), 7.22 (d, J=8.1 Hz, 2H), 6.59 (d, J=3.6 Hz, 1H), 2.34 (s, 3H) ppm. ¹³C NMR (126 MHz, CDCl₃): δ 145.39, 135.10, 133.65, 132.59, 130.11, 127.68, 127.57, 126.91, 124.16, 116.89, 115.07, 108.40, 21.71 ppm. The NMR spectra are in agreement with those reported in the literature.

Procedure for Determining the Rate of Gas Release to Solution. An oven-dried 10 mL round-bottom flask and a Schlenk adapter were cycled into a N₂-filled glovebox. In the glovebox, activated Mg₂(dobdc) was loaded into the round-bottom flask. The flask was sealed with the Schlenk adapter and removed from the glovebox. The flask containing Mg₂(dobdc) was connected to a Schlenk line and the VDF cylinder through a three-way valve. The three-way valve was used to pull vacuum on the regulator of the gas cylinder and to reactivate the MOF under high vacuum (<100 mTorr) while ramping the temperature to 300° C. (1° C./min) using a sand bath. The flask was allowed to stand at 300° C. under high vacuum (<100 mTorr) for 12 h. After activation, the flask was allowed to cool to room temperature under vacuum. Once cool, the joints of the flask containing the Mg₂(dobdc) were secured with copper wire, and the three-way valve was closed to the Schlenk line. The freshly activated sample of Mg₂(dobdc) was immediately dosed with approximately 1 bar of VDF at approximately 0° C. (in an ice-water bath) for 40 min.

While the sample of Mg₂(dobdc) was being dosed with VDF, three oven-dried Wilmad® screw-cap NMR tubes (Aldrich, p/n: Z271942-1EA) were evacuated and back-filled with N₂. This process was repeated a total of three times. Under active N₂ flow, anhydrous THE (450 μL) was added via syringe into each screw-cap NMR tube. The tubes were left under active N₂ flow until the reaction mixture was dispensed into the tube. Next, a 10 mL Schlenk flask equipped with a stir bar was flame-dried under vacuum.

After the 40 min dosing period, the flask containing the sample of freshly prepared VDF-Mg₂(dobdc) was closed under approximately 1 bar of VDF, and the VDF-Mg₂(dobdc) reagent was immediately brought into a N₂-filled glovebox. Once in the glovebox, the flask was placed in the freezer at −30° C. for 10 min. At this time, the freshly prepared VDF-Mg₂(dobdc) was weighed (190 mg VDF-Mg₂(dobdc), 34 wt % VDF, 1 mmol) and quickly transferred to a custom-built solid-addition funnel that enables solids to be added under air-free conditions and then controllably dispensed outside of a N₂-filled glovebox. The 10 mL Schlenk flask that had been flame-dried under vacuum was refilled with nitrogen. The funnel containing VDF-Mg₂(dobdc) was sealed, taken out of the box, and quickly attached to the 10 mL Schlenk flask under positive nitrogen. The funnel was used to dispense the MOF into the flask under static N₂. Anhydrous THE (6.0 mL) and Et₂O (2.0 mL) were added via syringe through the solid-addition funnel into the reaction flask. The solid-addition funnel was sealed. Through the sidearm of the Schlenk flask, fluorobenzene (94.2 μL, 1.00 mmol) was added. The mixture was allowed to stir at room temperature. Aliquots (50 μL) of the reaction mixture were removed via syringe and added to the NMR tubes at specified time intervals. The gas delivery was quantified by ¹⁹F NMR using fluorobenzene as an internal standard.

Pd-Catalyzed Defluorinative Coupling of Arylboronic Acids with VDF-Mg₂(dobdc).

TABLE 10
Optimization of Pd-catalyzed defluorinative coupling of VDF and PhBX₂ using VDF-Mg₂(dobdc).

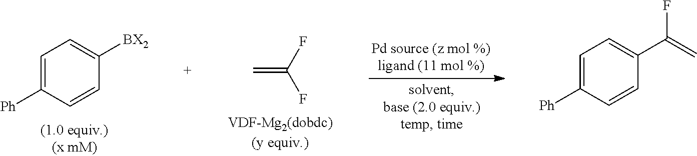

| | | Conc. | | Catalyst | | | Scale | VDF | | | | |
|---|---|---|---|---|---|---|---|---|---|---|---|---|
| Entry | PhBX₂ | (x mM) | Catalyst | (z mol %) | Ligand | Base | (mmol) | (y equiv.) | Solvent | Temp | Time | Yield^{a} |
| 1 | a | 100 | Pd(TFA)₂ | 10 | dtbbpy | — | 0.5 | 4 | DMF | rt | 24 h | 23% |
| 2 | a | 100 | Pd(TFA)₂ | 10 | dtbbpy | — | 0.5 | 4 | DMF | 50° C. | 24 h | 35% |
| 3 | a | 100 | Pd(TFA)₂ | 10 | dtbbpy | — | 0.5 | 4 | DMF | 80° C. | 24 h | 42% |
| 4 | a | 100 | Pd(TFA)₂ | 10 | dtbbpy | — | 0.5 | 4 | DMF | 120° C. | 24 h | 12% |
| 5 | a | 100 | Pd(TFA)₂ | 10 | dtbbpy | — | 0.5 | 4 | THF | 80° C. | 24 h | 43% |
| 6 | a | 100 | Pd(TFA)₂ | 10 | dtbbpy | — | 0.5 | 4 | toluene | 80° C. | 24 h | 24% |
| 7 | a | 100 | Pd(TFA)₂ | 10 | dtbbpy | — | 0.5 | 8 | DMF | 80° C. | 24 h | 47% |
| 8 | a | 100 | Pd(TFA)₂ | 10 | dtbbpy | — | 0.25 | 4 | THF | 80° C. | 24 h | 35% |
| 9 | b | 100 | Pd(TFA)₂ | 10 | dtbbpy | — | 0.25 | 4 | THF | 80° C. | 24 h | 0% |
| 10 | c | 100 | Pd(TFA)₂ | 10 | dtbbpy | — | 0.25 | 4 | THF | 80° C. | 24 h | 2% |
| 11 | a | 100 | Pd(TFA)₂ | 5 | dtbbpy | — | 0.25 | 4 | THF | 80° C. | 24 h | 23% |
| 12 | a | 100 | Pd(TFA)₂ | 20 | dtbbpy | — | 0.25 | 4 | THF | 80° C. | 24 h | 47% |
| 13 | a | 100 | Pd(TFA)₂ | 10 | dtbbpy | K₂CO₃ | 0.25 | 4 | THF | 80° C. | 24 h | 9% |
| 14 | a | 100 | Pd(TFA)₂ | 10 | dtbbpy | Cs₂CO₃ | 0.25 | 4 | THF | 80° C. | 24 h | 25% |
| 15 | a | 100 | Pd(TFA)₂ | 10 | dtbbpy | KOH | 0.25 | 4 | THF | 80° C. | 24 h | 13% |
| 16 | a | 100 | Pd(OAc)₂ | 10 | dtbbpy | — | 0.25 | 4 | THF | 80° C. | 24 h | 9% |
| 17 | a | 100 | Ni(PCy₃)₂Cl₂ | 10 | dtbbpy | — | 0.25 | 4 | THF | 80° C. | 24 h | 0% |
| 18 | a | 100 | Pd(TFA)₂(dtbbpy) | 10 | — | — | 0.25 | 4 | THF | 80° C. | 24 h | 42% |
| 19 | a | 100 | Pd(TFA)₂(dtbbpy) | 10 | — | — | 0.25 | 4 | THF | 80° C. | 48 h | 48% |
| 20 | a | 200 | Pd(TFA)₂(dtbbpy) | 10 | — | — | 0.25 | 4 | THF | 80° C. | 24 h | 21% |
| 21 | a | 50 | Pd(TFA)₂(dtbbpy) | 10 | — | — | 0.25 | 4 | THF | 80° C. | 24 h | 70% |
| 22 | a | 100 | Pd(TFA)₂(dtbbpy) | 10 | — | — | 0.25 | 4 | DMF | 80° C. | 24 h | 48% |
| 23 | a | 50 | Pd(TFA)₂(dibbpy) | 10 | — | — | 0.25 | 4 | DMF | 80° C. | 24 h | 100% |
| 24 | a | 50 | Pd(TFA)₂(dibbpy) | 10 | — | — | 0.25 | 8 | DMF | 80° C. | 24 h | 100% |

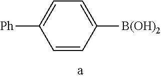 [SMILES: OB(O)c1ccc(-c2ccccc2)cc1]

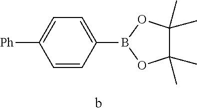 [SMILES: CC1(C)OB(c2ccc(-c3ccccc3)cc2)OC1(C)C]

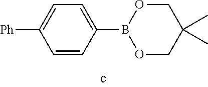 [SMILES: CC1(C)COB(c2ccc(-c3ccccc3)cc2)OC1]

^{a}Yields determined by ¹⁹F NMR using fluorobenzene as an internal standard. X mM refers to the concentration with respect to the arylboron species in the solvent. Optimal conditions are highlighted in blue.

Evaluation of Reaction Conditions (Table 10). In a N₂-filled glovebox, a 15 mL high-pressure reaction vessel was charged with a magnetic stir bar, aryl boronic acid (1.00 equiv.), Pd or Ni catalyst (5.0-20.0 mol %), ligand (11 mol %), and freshly prepared VDF-Mg₂(dobdc) (34 wt % VDF, 4-8 equiv.). Anhydrous DMF (5 mL) was added to the pressure vessel, and the vessel was quickly capped. The reaction was heated to the specified temperature and stirred for 24 h in the glovebox. After 24 h, the reaction was allowed to cool before fluorobenzene was added (47.1 μL, 500. Mmol). The reaction yields were determined by ¹⁹F NMR using fluorobenzene as an internal standard. The optimal conditions (entries 23-24) used pre-formed Pd(TFA)₂(dtbbpy) (10 mol %) as a catalyst under dilute conditions (50 mM) in DMF at 80° C. for 24 h with 4 or 8 equiv. of VDF-Mg₂(dobdc).

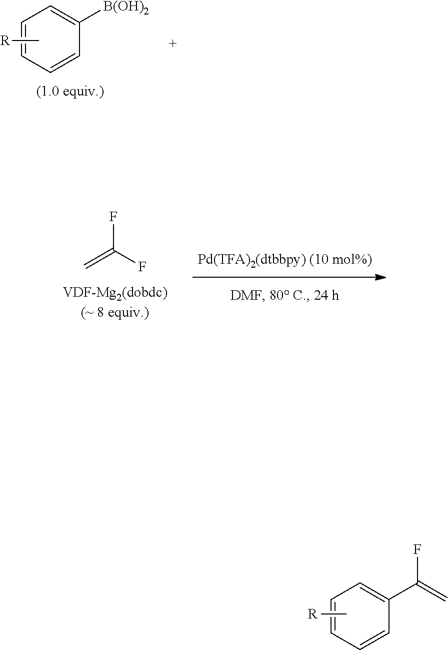

General Procedure 1. A 10 mL Schlenk flask was charged with a magnetic stir bar, (hetero)aryl boronic acid (250. Mmol, 1.00 equiv.), and Pd(TFA)₂(dtbbpy) (15.0 mg, 25.0 μmol, 10.0 mol %). The flask was evacuated and backfilled with N₂. This process was repeated a total of three times. In a N₂-filled glovebox, a custom-built, solid-addition funnel was loaded with freshly prepared Mg₂(dobdc) dosed with VDF (375 mg VDF-Mg₂(dobdc), 34 wt % VDF, −2 mmol, −8 equiv.). The funnel was sealed and removed from the glovebox. Under positive N₂ pressure, the glass stopper on the reaction flask was quickly replaced with the solid-addition funnel. The funnel was used to dispense the MOF into the reaction mixture under static N₂. Anhydrous DMF (5.0 mL) was added via syringe through the solid-addition funnel into the reaction flask. The solid-addition funnel was sealed. The reaction mixture was heated to 80° C. using a silicone oil bath and stirred for 24 h under static N₂. After 24 h, the reaction mixture was allowed to cool to room temperature and was vacuum-filtered to remove the MOF. The crude reaction mixture was purified by flash chromatography. The purified product was dried under high vacuum (<100 mTorr) for 12 h prior to characterization unless specified otherwise.

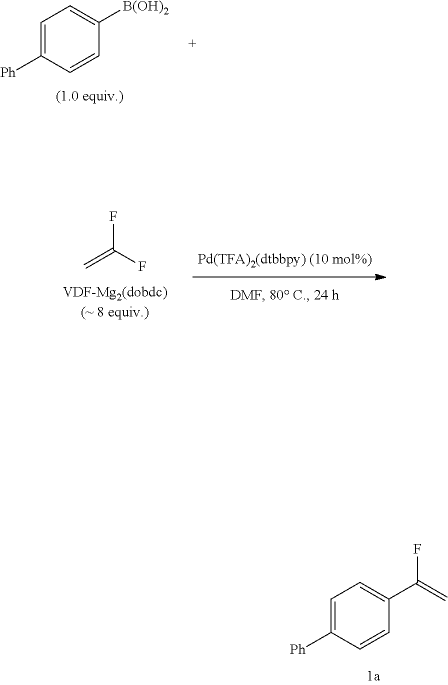

4-(1-fluorovinyl)-1,1′-biphenyl (1a). Prepared according to General Procedure 1. In a 10 mL Schlenk flask, 4-biphenylboronic acid (49.5 mg, 250. Mmol, 1.00 equiv.), Pd(TFA)₂(dtbbpy) (15.0 mg, 25.0 μmol, 10.0 mol %), VDF-Mg₂(dobdc) (375 mg VDF-Mg₂(dobdc), 34 wt % VDF, −2 mmol, −8 equiv.), and DMF (5.0 mL) were combined and stirred at 80° C. for 24 h. The crude reaction mixture was purified by flash chromatography using a Biotage Isolera instrument (SiO₂, pentane) to afford 4-(1-fluorovinyl)-1,1′-biphenyl (29.9 mg, 61%) as a white solid. ¹H NMR (500 MHz, CDCl₃): δ 7.69-7.61 (m, 6H), 7.52-7.45 (m, 2H), 7.43-7.36 (m, 1H), 5.10 (dd, J=49.7, 3.6 Hz, 1H), 4.91 (dd, J=17.9, 3.5 Hz, 1H) ppm. ¹³C NMR (126 MHz, CDCl₃): δ 163.15 (d, J=249.8 Hz), 142.50, 140.65, 131.26 (d, J=29.4 Hz), 129.26, 128.09, 127.54 (d, J=2.1 Hz), 127.43, 125.43 (d, J=7.0 Hz), 89.97 (d, J=22.5 Hz) ppm. ¹⁹F NMR (376 MHz, CDCl₃): δ −107.84 (dd, J=49.7, 17.8 Hz) ppm. The NMR spectra are in agreement with those reported in the literature. Observed HRMS (DART-MS): [M+H]⁺=199.08600, Predicted [M+H]⁺=199.08783.

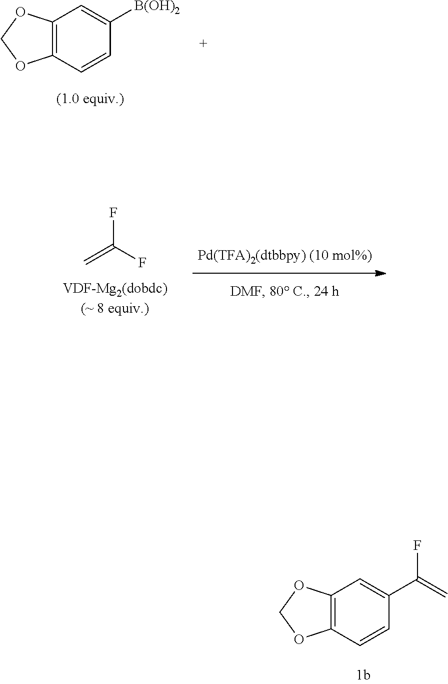

5-(1-fluorovinyl)benzo[d][1,3]dioxole (1b). Prepared according to General Procedure 1. In a 10 mL Schlenk flask, 3,4-(methylenedioxy)phenylboronic acid (41.5 mg, 250. Mmol, 1.00 equiv.), Pd(TFA)₂(dtbbpy) (15.0 mg, 25.0 μmol, 10.0 mol %), VDF-Mg₂(dobdc) (375 mg VDF-Mg₂(dobdc), 34 wt % VDF, −2 mmol, −8 equiv.), and DMF (5.0 mL) were combined and stirred at 80° C. for 24 h. The crude reaction mixture was purified by flash chromatography using a Biotage Isolera instrument (0%→5% CH₂Cl₂ in pentane) to afford 5-(1-fluorovinyl)benzo[d][1,3]dioxole (23.7 mg, 57%) as a clear liquid. Due to its volatility, the product was dried under high vacuum (>100 mtorr) for 2 h prior to characterization. ¹H NMR (500 MHz, CDCl₃): δ 7.08 (dd, J=8.1, 1.8 Hz, 1H), 7.00 (d, J=1.8 Hz, 1H), 6.80 (d, J=8.2 Hz, 1H), 5.99 (s, 2H), 4.87 (dd, J=49.8, 3.5 Hz, 1H), 4.75 (dd, J=18.0, 3.5 Hz, 1H) ppm. ¹³C NMR (126 MHz, CDCl₃): δ 162.75 (d, J=249.3 Hz), 148.73, 148.01 (d, J=2.6 Hz), 126.33 (d, J=29.9 Hz), 119.05 (d, J=7.6 Hz), 108.35 (d, J=1.7 Hz), 105.31 (d, J=7.6 Hz), 101.54, 88.32 (d, J=23.0 Hz) ppm. ¹⁹F NMR (376 MHz, CDCl₃): δ −106.11 (dd, J=49.8, 18.1 Hz) ppm. Observed HRMS (DART-MS): [M+H]+=167.04932, Predicted [M+H]⁺=167.04636. IR (neat, cm⁻¹): 2897 (w), 2782 (w), 1649 (m), 1609 (w), 1504 (w), 1489 (s), 1441 (m), 1353 (m), 1284 (m), 1253 (m), 1230 (s), 1159 (w), 1107 (m), 1036 (s), 929 (s), 898 (m), 836 (w), 809 (s), 729 (w), 711 (w), 653 (m), 552 (w), 529 (m).

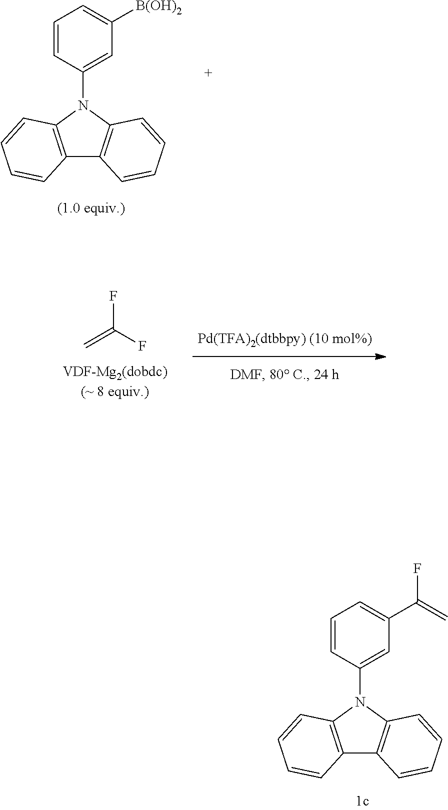

9-(3-(1-fluorovinyl)phenyl)-9H-carbazole (1c). Prepared according to General Procedure 1. In a 10 mL Schlenk flask, (3-(9H-carbazol-9-yl)phenyl)boronic acid (71.8 mg, 250. Mmol, 1.00 equiv.), Pd(TFA)₂(dtbbpy) (15.0 mg, 25.0 μmol, 10.0 mol %), VDF-Mg₂(dobdc) (375 mg VDF-Mg₂(dobdc), 34 wt % VDF, −2 mmol, −8 equiv.), and DMF (5.0 mL) were combined and stirred at 80° C. for 24 h. The crude reaction mixture was purified by flash chromatography using a Biotage Isolera instrument (SiO₂, gradient of 0%→8% CH₂Cl₂ in pentane) to afford 9-(3-(1-fluorovinyl)phenyl)-9H-carbazole (21.5 mg, 30%) as a clear oil. ¹H NMR (500 MHz, CDCl₃): δ 8.16 (d, J=7.8 Hz, 2H), 7.77 (s, 1H), 7.70-7.61 (m, 2H), 7.58 (dt, J=7.6, 1.8 Hz, 1H), 7.47-7.38 (m, 4H), 7.34-7.29 (m, 2H), 5.13 (dd, J=49.2, 3.7 Hz, 1H), 4.97 (dd, J=17.7, 3.7 Hz, 1H) ppm. ¹⁹F NMR (376 MHz, CDCl₃): δ 15-107.69 (dd, J=49.2, 17.6 Hz) ppm. ¹³C NMR (126 MHz, CDCl₃): δ 162.13 (d, J=250.7 Hz), 140.88, 138.27 (d, J=2.4 Hz), 134.12 (d, J=29.8 Hz), 130.27 (d, J=2.0 Hz), 128.13, 126.21, 123.71 (d, J=6.9 Hz), 123.60, 123.46 (d, J=7.1 Hz), 120.53, 120.28, 109.78, 90.95 (d, J=22.2 Hz). Observed HRMS (DART-MS): [M+H]⁺=288.11581, Predicted [M+H]+=288.11438. IR (neat, cm⁻¹): 3054 (w), 2924 (w), 2850 (w), 1651 (w), 1584 (m), 1495 (m), 1477 (w), 1450 (s), 1359 (w), 1334 (w), 1313 (m), 1297 (w), 1281 (w), 1264 (w), 1228 (m), 1177 (w), 1148 (w), 1119 (w), 1076 (w), 1026 (w), 1016 (w), 1002 (w), 975 (w), 924 (m), 908 (w), 845 (m), 796 (m), 746 (s), 723 (m), 689 (w), 670 (w), 638 (w), 617 (w), 562 (w), 528 (w).

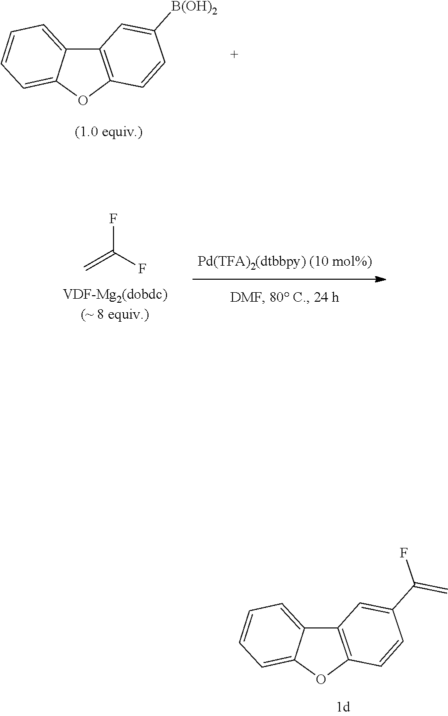

2-(1-fluorovinyl)dibenzo[b,d]furan (1d). Prepared according to General Procedure 1. In a 10 mL Schlenk flask, dibenzo[b,d]furan-2-ylboronic acid (53.0 mg, 250. Mmol, 1.00 equiv.), Pd(TFA)₂(dtbbpy) (15.0 mg, 25.0 μmol, 10.0 mol %), VDF-Mg₂(dobdc) (375 mg VDF-Mg₂(dobdc), 34 wt % VDF, −2 mmol, −8 equiv.), and DMF (5.0 mL) were combined and stirred at 80° C. for 24 h. The crude reaction mixture was purified by flash chromatography using a Biotage Isolera instrument (SiO₂, pentane) to afford 2-(1-fluorovinyl)dibenzo[b,d]furan (30.2 mg, 57%) as a clear oil. ¹H NMR (500 MHz, CDCl₃): δ 8.15 (d, J=1.9 Hz, 1H), 7.97 (d, J=7.6 Hz, 1H), 7.67 (dd, J=8.7, 1.9 Hz, 1H), 7.57 (q, J=8.5 Hz, 2H), 7.49 (td, J=7.3, 1.4 Hz, 1H), 7.40-7.35 (m, 1H), 5.09 (dd, J=50.0, 3.5 Hz, 1H), 4.90 (dd, J=18.1, 3.5 Hz, 1H) ppm. ¹³C NMR (126 MHz, CDCl₃): δ 163.44 (d, J=249.8 Hz), 156.82, 156.79, 128.04, 127.44 (d, J=29.5 Hz), 124.84 (d, J=1.8 Hz), 124.49 (d, J=7.1 Hz), 124.23, 123.44, 121.20, 117.52 (d, J=7.3 Hz), 112.23, 112.10 (d, J=2.1 Hz), 89.36 (d, J=22.9 Hz) ppm. ¹⁹F NMR (376 MHz, CDCl₃): δ −105.80 (dd, J=49.9, 18.0 Hz) ppm. Observed HRMS (DART-MS): [M+H]⁺=213.07507, Predicted [M+H]⁺=213. 06710. IR (neat, cm⁻¹): 2925 (w), 1804 (w), 1650 (w), 1603 (w), 1477 (w), 1450 (w), 1431 (w), 1346 (w), 1322 (w), 1308 (w), 1283 (w), 1258 (w), 1198 (m), 1150 (w), 1126 (w), 1107 (w), 1077 (w), 1022 (w), 926 (w), 889 (w), 874 (w), 840 (w), 815 (w), 796 (w), 766 (w), 747 (w), 732 (w), 717 (w), 651 (w), 620 (w), 536 (w).

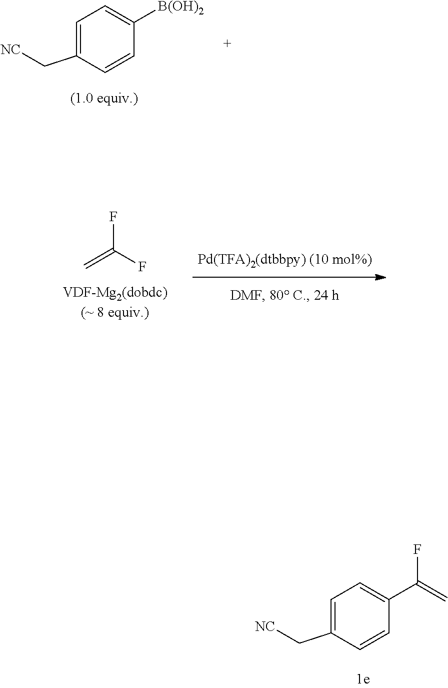

1-(1-fluorovinyl)-4-phenoxybenzene (1e). Prepared according to General Procedure 1. In a 10 mL Schlenk flask, (4-(cyanomethyl)phenyl)boronic acid (40.2 mg, 250. Mmol, 1.00 equiv.), Pd(TFA)₂(dtbbpy) (15.0 mg, 25.0 μmol, 10.0 mol %), VDF-Mg₂(dobdc) (375 mg VDF-Mg₂(dobdc), 34 wt % VDF, −2 mmol, −8 equiv.), and DMF (5.0 mL) were combined and stirred at 80° C. for 24 h. The crude reaction mixture was purified by flash chromatography using a Biotage Isolera instrument (SiO₂, 0%→50% CH₂Cl₂ in pentane) to afford 2-(4-(1-fluorovinyl)phenyl)acetonitrile (12.3 mg, 31%) as a white solid. ¹H NMR (400 MHz, CDCl₃): δ 7.57 (d, J=8.1 Hz, 2H), 7.35 (d, J=8.0 Hz, 2H), 5.06 (dd, J=49.4, 3.6 Hz, 1H), 4.89 (dd, J=17.7, 3.6 Hz, 1H), 3.77 (s, 2H). ¹³C NMR (126 MHz, CDCl₃): δ 162.26 (d, J=250.2 Hz), 132.08 (d, J=29.7 Hz), 131.12, 128.23 (d, J=2.1 Hz), 125.48 (d, J=7.0 Hz), 117.57, 90.45 (d, J=22.3 Hz), 23.58. ¹⁹F NMR (376 MHz, CDCl₃): a −108.03 (dd, J=49.5, 17.7 Hz). Observed HRMS (DART-MS): [M+H]⁺=162.06525, Predicted [M+H]⁺=162.06743. Melting Point: 60.0-62.0° C. IR (neat, cm⁻¹): 2939 (w), 2919 (w), 2252 (w), 1649 (w), 1611 (w), 1567 (w), 1510 (w), 1408 (w), 1372 (w), 1312 (w), 1277 (w), 1265 (w), 1180 (w), 1122 (w), 1090 (w), 1015 (w), 978 (w), 961 (w), 918 (m), 847 (m), 814 (m), 722 (w), 701 (w), 604 (w), 579 (w).

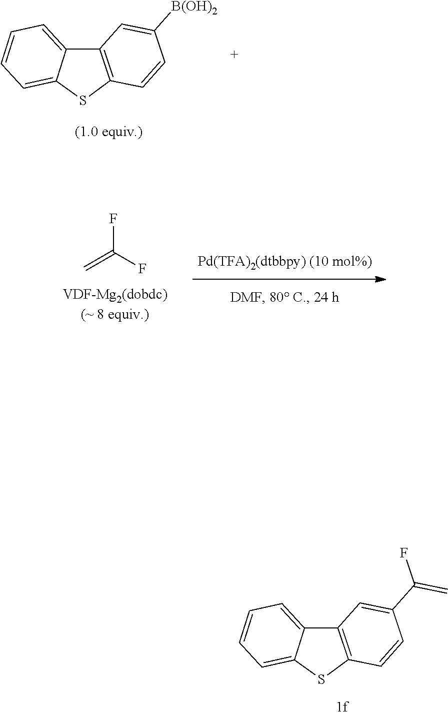

2-(1-fluorovinyl)dibenzo[b,d]thiophene (1f). Prepared according to General Procedure 1. In a 10 mL Schlenk flask, dibenzo[b,d]thiophen-2-ylboronic acid (57.0 mg, 250. Mmol, 1.00 equiv.), Pd(TFA)₂(dtbbpy) (15.0 mg, 25.0 μmol, 10.0 mol %), VDF-Mg₂(dobdc) (375 mg VDF-Mg₂(dobdc), 34 wt % VDF, −2 mmol, −8 equiv.), and DMF (5.0 mL) were combined and stirred at 80° C. for 24 h. The crude reaction mixture was purified by flash chromatography using a Biotage Isolera instrument (SiO₂, pentane) to afford 2-(1-fluorovinyl)dibenzo[b,d]thiophene (18.8 mg, 33%) as a white solid. ¹H NMR (500 MHz, CDCl₃): δ 8.33 (d, J=1.8 Hz, 1H), 8.22-8.16 (m, 1H), 7.91-7.81 (m, 2H), 7.64 (dd, J=8.3, 1.8 Hz, 1H), 7.49 (dt, J=6.1, 3.7 Hz, 2H), 5.16 (dd, J=49.9, 3.6 Hz, 1H), 4.95 (dd, J=18.0, 3.6 Hz, 1H) ppm. ¹³C NMR (126 MHz, CDCl₃): δ 163.13 (d, J=249.8 Hz), 140.61, 139.94, 135.79 (d, J=1.7 Hz), 135.34, 128.64 (d, J=29.3 Hz), 127.27, 124.77, 123.32 (d, J=7.0 Hz), 123.03, 122.93 (d, J=2.3 Hz), 121.88, 117.72 (d, J=7.3 Hz), 89.62 (d, J=22.7 Hz) ppm. ¹⁹F NMR (376 MHz, CDCl₃): δ −106.98 (dd, J=49.9, 18.0 Hz) ppm. Observed HRMS (DART-MS): [M+H]⁺=228.03922, Predicted [M+H]⁺=228.040900. Melting Point: 49.6-50.9° C. IR (neat, cm⁻¹): 3050 (w), 1805 (w), 1649 (s), 1600 (w), 1476 (m), 1451 (m), 1423 (w), 1347 (w), 1308 (m), 1284 (w), 1255 (m), 1198 (m), 1150 (w), 1126 (w), 1106 (w), 1073 (w), 1022 (w), 923 (m), 897 (w), 885 (w), 874 (w), 840 (w), 822 (m), 796 (w), 769 (w), 750 (m), 735 (w), 716 (w), 654 (m), 628 (w), 557 (w), 535 (w).

Pd-Catalyzed Negishi Coupling of Aryl Halides with VDF-ZnCl-TMEDA.

TABLE 11
Optimization of Pd-catalyzed Negishi coupling of VDF-ZnCl•TMEDA and aryl halides.

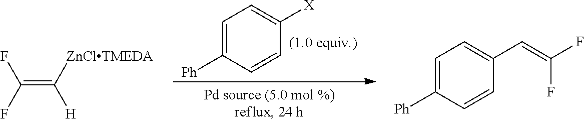

| Entry | X | Catalyst | Solvent | Temp | Time (h) | Yield^{a} |
|---|---|---|---|---|---|---|
| 1 | I | Pd(PPh₃)₄ | THF/Et₂O | 55° C. | 24 h | 53% |
| 2 | Br | Pd(PPh₃)₄ | THF/Et₂O | 55° C. | 24 h | 49% |
| 3 | Br | XPhos Pd G3 | THF/Et₂O | 55° C. | 24 h | 88% |

^{a}Yields determined by ¹⁹F NMR using fluorobenzene as an internal standard.

Evaluation of Reaction Conditions (Table 11). Adapted from a previous literature procedure. A 10 ml Schlenk flask equipped with a stir bar was flame-dried under vacuum and allowed to cool under vacuum. In a N₂-filled glovebox, a custom-built, solid-addition funnel was loaded with freshly prepared Mg₂(dobdc) dosed with VDF (375 mg VDF-Mg₂(dobdc), 34 wt % VDF, −2 mmol, −4 equiv.). The funnel was sealed and removed from the glovebox. Under positive N₂ pressure, the glass stopper on the reaction flask was quickly replaced with the solid-addition funnel. Under static N₂, the funnel was used to dispense the MOF into the reaction flask. Anhydrous THE (6.0 mL) and anhydrous Et₂O (2.0 mL) were added via syringe to the reaction flask through the solid-addition funnel. The solid-addition funnel was closed, and the MOF was stirred in the solution for 40 min. After 40 min, the solution was cooled to −110° C. using an EtOH/liquid N₂ bath. At −110° C., anhydrous tetramethylethylenediamine (TMEDA) (0.20 mL, 1.3 mmol, 2.6 equiv.) was added through the side arm of the Schlenk flask, and the solution was stirred for 15 min. Still at −110° C., a solution of sec-BuLi (1.4 M in cyclohexane, 0.72 mL, 1.0 mmol, 2.0 equiv.) was added through the side arm of the Schlenk flask. The mixture was stirred at −110° C. for 60 min. Still at −110° C., a solution of anhydrous ZnCl₂ (1.0 M in Et₂O, 1.2 mL, 1.2 mmol, 2.4 equiv.) was added to the reaction mixture. The reaction mixture was allowed to warm to −100° C. and stirred for 90 min. Under flowing N₂, the reaction mixture was allowed to warm to room temperature. Once at room temperature, the funnel was removed so that the aryl halide (500. Mmol, 1.00 equiv.) and Pd catalyst (25.0 μmol, 5.00 mol %) could be quickly added under positive N₂ flow. Then, the flask was sealed using an oven-dried reflux condenser. The reaction was refluxed under static N₂ for 24 h. The reaction was allowed to cool to room temperature before adding fluorobenzene (47.1 μL, 500. Mmol). The reaction yields were determined by ¹⁹F NMR using fluorobenzene as an internal standard. The use of aryl bromides with Xphos Pd G3 as the catalyst was found to be optimal.

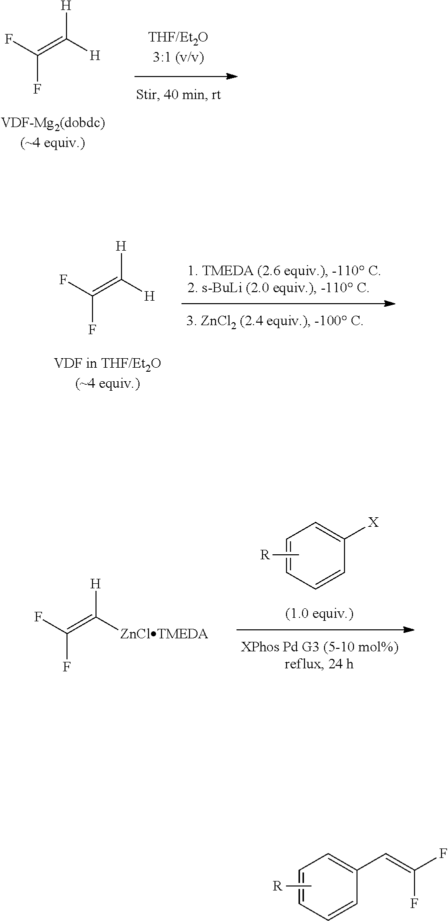

General Procedure 2. A 10 ml Schlenk flask equipped with a stir bar was flame-dried under vacuum. In a N₂-filled glovebox, a custom-built, solid-addition funnel was loaded with freshly prepared Mg₂(dobdc) dosed with VDF (375 mg VDF-Mg₂(dobdc), 34 wt % VDF, −2 mmol, −4 equiv.). The funnel was sealed and removed from the glovebox. Under positive N₂ pressure, the glass stopper on the reaction flask was quickly replaced with the solid-addition funnel. Under static N₂, the funnel was used to dispense the MOF into the reaction flask. Anhydrous THE (6.0 mL) and anhydrous Et₂O (2.0 mL) were added via syringe to the reaction flask through the solid-addition funnel. The solid-addition funnel was closed and the MOF was stirred in the solution for 40 min. After 40 min, the solution was cooled to −110° C. using an EtOH/liquid N₂ bath. At −110° C., anhydrous TMEDA (0.20 mL, 1.3 mmol, 2.6 equiv.) was added through the side arm of the Schlenk flask, and the solution was stirred for 15 min. Still at −110° C., a solution of sec-BuLi (1.4 M in cyclohexane, 0.72 mL, 1.0 mmol, 2.0 equiv.) was added through the side arm of the Schlenk flask. The mixture was stirred at −110° C. for 60 min. Still at −110° C., a solution of anhydrous ZnCl₂ (1.0 M in Et₂O, 1.2 mL, 1.2 mmol, 2.4 equiv.) was added to the reaction mixture. The reaction mixture was allowed to warm to −100° C. and stirred for 90 min. Under N₂, the reaction mixture was allowed to warm to room temperature. Once at room temperature, the funnel was removed so that the aryl halide (500. Mmol, 1.00 equiv.) and Xphos Pd G3 (21.2 mg, 25.0 μmol, 5.00 mol %) could be quickly added under positive N₂ flow. Then, the flask was sealed using an oven-dried reflux condenser. The reaction was refluxed under static N₂ for the specified time. After the reaction was complete, the crude reaction mixture was allowed to cool to room temperature, quenched with water (2 mL), and vacuum-filtered to remove the MOF. The aqueous phase was extracted with dichloromethane (3×15 mL). The combined organic layers were concentrated under reduced pressure and purified by flash chromatography using a Biotage Isolera instrument. The purified product was dried under high vacuum (<100 mTorr) for 12 h unless specified otherwise prior to characterization.

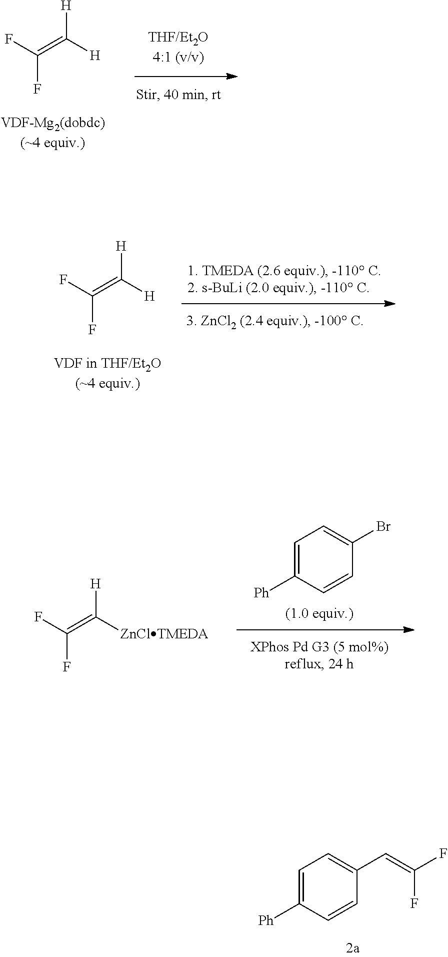

4-(2,2-difluorovinyl)-1,1′-biphenyl (2a). Prepared according to General Procedure 2. To a 10 mL Schlenk flask containing the 2,2-difluorovinylzinc chloride-TMEDA complex (−280 mg, 1 mmol, 2 equiv. generated in situ), 4-bromo-1,1′-biphenyl (116.6 mg, 500.0 μmol, 1.000 equiv.) and Xphos Pd G3 (25.0 μmol, 5.00 mol %) were quickly added under positive N₂ flow. The reaction was refluxed under static N₂ for 24 h. The crude product was purified by flash chromatography using a Biotage Isolera instrument (SiO₂, pentane) to afford 4-(2,2-difluorovinyl)-1,1′-biphenyl (84.3 mg, 78%) as a white solid. ¹H NMR (500 MHz, CDCl₃): δ 7.60 (t, J=1.7 Hz, 4H), 7.51-7.40 (m, 4H), 7.36 (t, J=3.1 Hz, 1H), 5.33 (dd, J=26.3, 3.7 Hz, 1H) ppm. ¹³C NMR (126 MHz, CDCl₃): δ 156.49 (dd, J=298.5, 288.5 Hz), 140.63, 139.94 (t, J=2.1 Hz), 129.51 (t, J=6.4 Hz), 128.97, 128.13 (dd, J=6.4, 3.6 Hz), 127.56, 127.49, 127.09, 82.06 (dd, J=29.2, 13.6 Hz) ppm. ¹⁹F NMR (376 MHz, CDCl₃): δ −81.89 (dd, J=30.6, 26.3 Hz), −83.81 (dd, J=30.7, 3.7 Hz) ppm. The NMR spectra are in agreement with those reported in the literature. Observed HRMS (DART-MS): [M+H]⁺=217.07774, Predicted [M+H]⁺=217.07841.

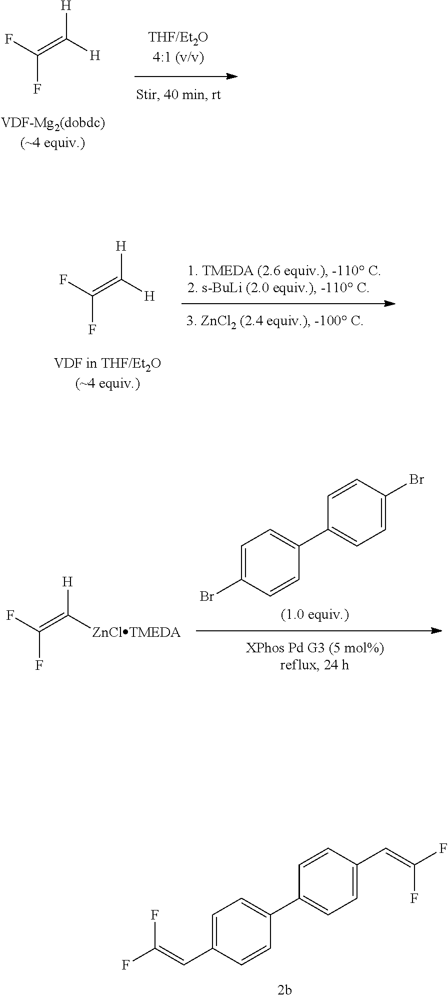

4,4′-bis(2,2-difluorovinyl)-1,1′-biphenyl (2b). Prepared according to General Procedure 2. To a 10 mL Schlenk flask containing the 2,2-difluorovinylzinc chloride-TMEDA complex (−280 mg, 1 mmol, 2 equiv. generated in situ), 4,4′-dibromo-1,1′-biphenyl (78.0 mg, 250. Mmol, 0.500 equiv.) and Xphos Pd G3 (25.0 μmol, 5.00 mol %) were quickly added under positive N₂ flow. The reaction was refluxed under static N₂ for 24 h. The crude product was purified by flash chromatography using a Biotage Isolera instrument (SiO₂, pentane) to afford 4,4′-bis(2,2-difluorovinyl)-1,1′-biphenyl (43.8 mg, 63%) as a white solid. ¹H NMR (500 MHz, CDCl₃): δ 7.58 (d, J=8.4 Hz, 4H), 7.41 (d, J=8.3 Hz, 4H), 5.32 (dd, J=26.3, 3.7 Hz, 2H) ppm. ¹³C NMR (126 MHz, CDCl₃): δ 156.52 (dd, J=298.6, 288.7 Hz), 139.16 (t, J=2.1 Hz), 129.70 (t, J=6.4 Hz), 128.19 (dd, J=6.5, 3.5 Hz), 127.25, 82.05 (dd, J=29.3, 13.6 Hz) ppm. ¹⁹F NMR (376 MHz, CDCl₃): δ −81.75 (dd, J=30.3, 26.3 Hz), −83.66 (dd, J=30.2, 3.7 Hz) ppm. The NMR spectra are in agreement with those reported in the literature. Observed HRMS (DART-MS): [M+H]⁺=279.07422, Predicted [M+H]⁺=279.07522.

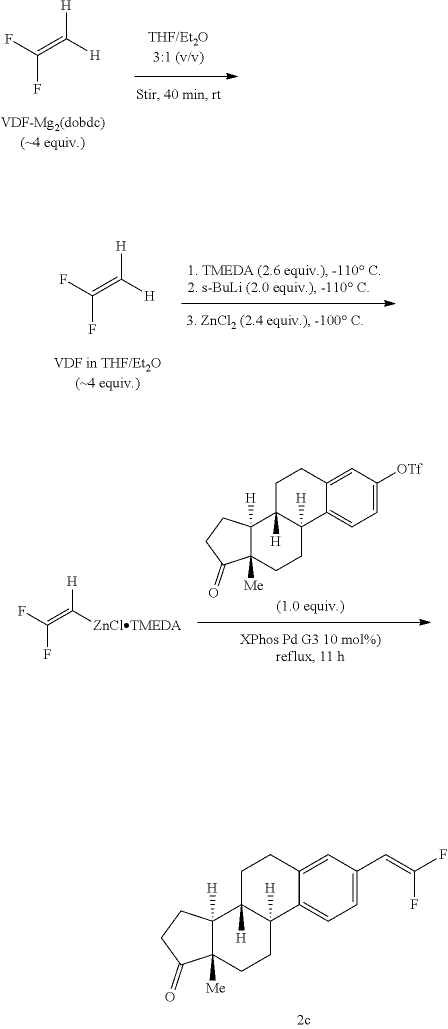

(8R,9S,13R)-3-(2,2-difluorovinyl)-13-methyl-6,7,8,9,11,12,13,14,15,16-decahydro-17H-cyclopenta[a]-68-henanthrene-17-one (2c). Prepared according to General Procedure 2. To a 10 mL Schlenk flask containing the 2,2-difluorovinylzinc chloride-TMEDA complex (−280 mg, 1 mmol, 2 equiv. generated in situ), (8R,9S,13R)-13-methyl-17-oxo-7,8,9,11,12,13,14,15,16,17-decahydro-6H-cyclopenta[a]-68-henanthrene-3-yl trifluoromethanesulfonate (201.2 mg, 500.0 μmol, 1.000 equiv.) and Xphos Pd G3 (25.0 μmol, 5.00 mol %) were quickly added under positive N₂ flow. The reaction was refluxed under static N₂ for 11 h. The crude product was purified by flash chromatography using a Biotage Isolera instrument (SiO₂, gradient of 0%→80% CH₂Cl₂ in hexanes) to afford (8R,9S,13R)-3-(2,2-difluorovinyl)-13-methyl-6,7,8,9,11,12,13,14,15,16-decahydro-17H-cyclopenta[a]-68-henanthrene-17-one (73.9 mg, 47%) as a white sticky solid. ¹H NMR (500 MHz, CDCl₃): δ 7.29 (d, J=8.0 Hz, 1H), 7.15 (d, J=8.2 Hz, 1H), 7.09 (s, 1H), 5.24 (dd, J=26.5, 3.8 Hz, 1H), 2.93 (dd, J=9.2, 4.3 Hz, 2H), 2.54 (dd, J=19.0, 8.8 Hz, 1H), 2.44 (dt, J=8.8, 3.9 Hz, 1H), 2.32 (td, J=10.9, 4.2 Hz, 1H), 2.18 (dt, J=18.5, 8.9 Hz, 1H), 2.12-2.02 (m, 2H), 2.02-1.97 (m, 1H), 1.72-1.41 (m, 6H), 0.94 (s, 3H). ¹³C NMR (126 MHz, CDCl₃): δ 220.91, 156.30 (dd, J=297.9, 287.7 Hz), 138.88 (t, J=2.0 Hz), 136.95, 128.28 (dd, J=6.0, 3.6 Hz), 127.92 (t, J=6.3 Hz), 125.83, 125.18 (dd, J=6.2, 3.4 Hz), 81.89 (dd, J=28.8, 13.7 Hz), 50.61, 48.10, 44.46, 38.23, 35.97, 31.69, 29.47, 26.56, 25.78, 21.70, 13.95. ¹⁹F NMR (376 MHz, CDCl₃): δ −81.05 (dd, J=28.4, 25.7 Hz), −82.77 (dd, J=28.3, 3.5 Hz) ppm. Observed HRMS (DART-MS): [M+H]⁺=317.17032, Predicted [M+H]⁺=317.16723. Melting Point: 126.0-128.0° C. IR (neat, cm⁻¹): 2956 (w), 2923 (w), 2857 (w), 1731 (m), 1502 (w), 1472 (w), 1451 (w), 1434 (w), 1408 (w), 1374 (w), 1345 (w), 1279 (w), 1258 (w), 1244 (w), 1225 (w), 1195 (w), 1174 (w), 1153 (w), 1084 (w), 1054 (w), 1038 (w), 1007 (w), 963 (w), 943 (w), 927 (w), 910 (w), 888 (w), 874 (w), 843 (w), 821 (w), 792 (w), 769 (w), 756 (w), 738 (w), 738 (w), 723 (w), 708 (w), 695 (w), 664 (w), 632 (w), 610 (w), 581 (w), 557 (w), 537 (w), 517 (w).

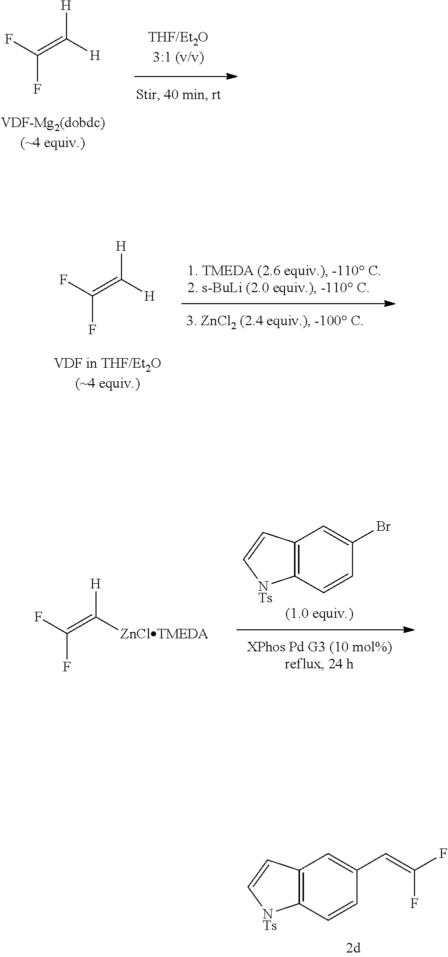

5-(2,2-difluorovinyl)-1-tosyl-1H-indole (2d). Prepared according to General Procedure 2. To a 10 mL Schlenk flask containing the 2,2-difluorovinylzinc chloride-TMEDA complex (−280 mg, 1 mmol, 2 equiv. generated in situ), 5-bromo-1-tosyl-1H-indole (175.1 mg, 500.0 μmol, 1.000 equiv.) and Xphos Pd G3 (42.4 mg, 50.0 μmol, 10.0 mol %) were quickly added under positive N₂ flow. The reaction was refluxed under static N₂ for 24 h. The crude product was purified by flash chromatography using a Biotage Isolera instrument (SiO₂, gradient of 0%→25% CH₂Cl₂ in pentane) to afford 5-(2,2-difluorovinyl)-1-tosyl-1H-indole (118.4 mg, 71%) as a clear oil. ¹H NMR (500 MHz, CDCl₃): δ 8.01 (d, J=8.7 Hz, 1H), 7.85-7.78 (m, 2H), 7.61 (d, J=3.7 Hz, 1H), 7.53 (s, 1H), 7.32 (dd, J=8.6, 1.8 Hz, 1H), 7.26-7.23 (m, 2H), 6.69-6.63 (m, 1H), 5.36 (dd, J=26.1, 3.9 Hz, 1H), 2.36 (s, 3H) ppm. ¹³C NMR (126 MHz, CDCl₃): δ 156.17 (dd, J=297.3, 287.5 Hz), 145.18, 135.27, 133.77 (d, J=2.0 Hz), 131.27, 130.01, 127.13, 126.88, 125.64 (t, J=6.3 Hz), 124.48 (dd, J=6.0, 3.2 Hz), 120.44 (dd, J=6.9, 3.7 Hz), 113.82, 109.07, 82.17 (dd, J=29.3, 13.7 Hz), 21.62 ppm. ¹⁹F NMR (376 MHz, CDCl₃): δ −83.48 (dd, J=60.9, 26.0 Hz), −84.96 (dd, J=34.1, 3.9 Hz) ppm. Observed HRMS (DART-MS): [M+H]⁺=334.06888, Predicted [M+H]+=334.06686. IR (neat, cm⁻¹): 1728 (s), 1596 (w), 1494 (w), 1460 (w), 1444 (w), 1371 (m), 1324 (w), 1276 (m), 1237 (w), 1171 (m), 1143 (w), 1128 (s), 1093 (w), 996 (m), 971 (w), 922 (w), 905 (w), 888 (w), 831 (w), 812 (m), 766 (w), 724 (w), 703 (w), 678 (m), 666 (w), 626 (w), 582 (s), 563 (w), 538 (m).

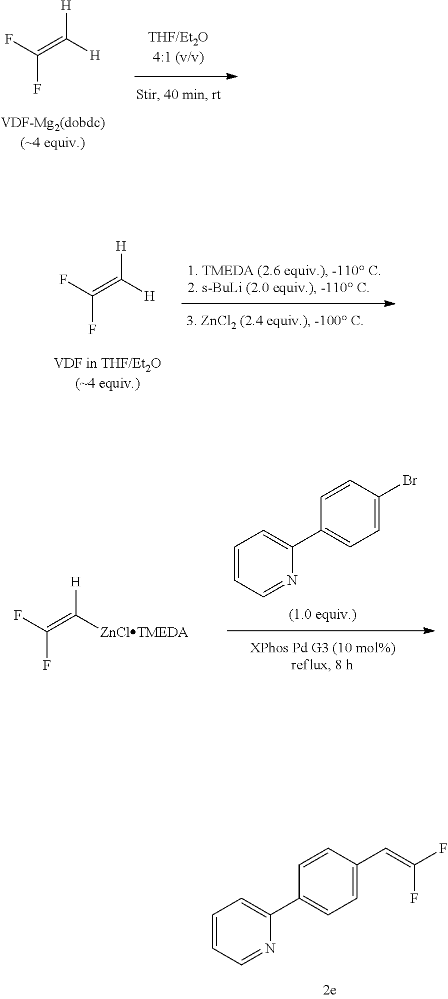

2-(4-(2,2-difluorovinyl)phenyl)pyridine (2e). Prepared according to General Procedure 2. To a 10 mL Schlenk flask containing the 2,2-difluorovinylzinc chloride-TMEDA complex (−280 mg, 1 mmol, 2 equiv. generated in situ), 2-(4-bromophenyl)pyridine (117.0 mg, 500.0 μmol, 1.000 equiv.) and Xphos Pd G3 (42.4 mg, 50.0 μmol, 10.0 mol %) were quickly added under positive N₂ flow. The reaction was refluxed under static N₂ for 8 h. The crude product was purified by flash chromatography using a Biotage Isolera instrument (SiO₂, CH₂Cl₂) to afford 2-(4-(2,2-difluorovinyl)phenyl)pyridine (46.8 mg, 43%) as a white solid. ¹H NMR (500 MHz, CDCl₃): δ 8.69 (dd, J=4.7, 1.8 Hz, 1H), 7.98 (d, J=8.0 Hz, 2H), 7.78-7.68 (m, 2H), 7.44 (d, J=8.0 Hz, 2H), 7.23 (ddt, J=6.5, 4.8, 1.5 Hz, 1H), 5.33 (dd, J=26.3, 3.7 Hz, 1H). ¹³C NMR (126 MHz, CDCl₃): δ 156.88, 156.58 (dd, J=299.88, 289.80 Hz), 149.86, 138.08 (t, J=2.2 Hz), 136.90, 131.20 (t, J=6.5 Hz), 128.08 (dd, J=6.5, 3.6 Hz), 127.26, 122.31, 120.46, 82.17 (dd, J=29.3, 13.5 Hz) ppm. ¹⁹F NMR (376 MHz, CDCl₃): δ −81.15 (dd, J=29.1, 26.2 Hz), −83.21 (dd, J=29.0, 3.5 Hz) ppm. The NMR spectra are in agreement with those reported in the literature. Observed HRMS (DART-MS): [M+H]⁺=218.07680, Predicted [M+H]⁺=218.07366.

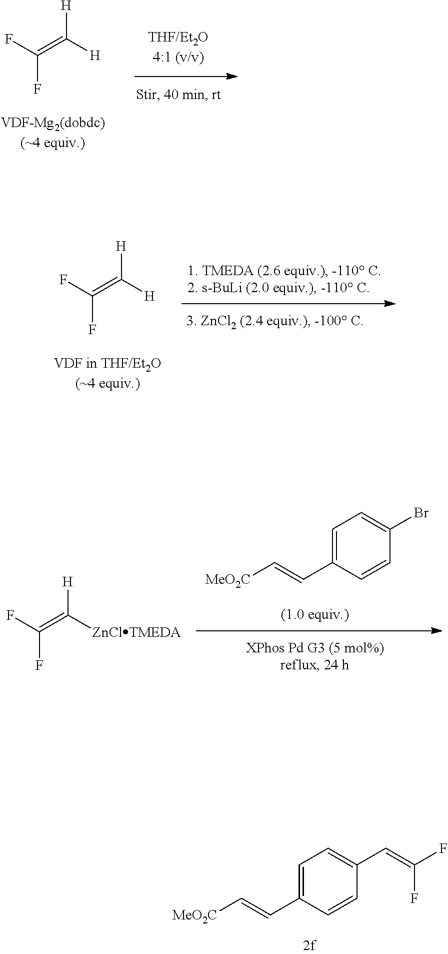

Methyl-3(E)-(4-(2,2-difluorovinyl)phenyl)acrylate (2f). Prepared according to General Procedure 2. To a 10 mL Schlenk flask containing the 2,2-difluorovinylzinc chloride-TMEDA complex (−280 mg, 1 mmol, 2 equiv. generated in situ), methyl—3(E)-(4-bromophenyl)acrylate (120.5 mg, 500.0 μmol, 1.000 equiv.) and Xphos Pd G3 (21.2 mg, 25.0 μmol, 5.00 mol %) were quickly added under positive N₂ flow. The reaction was refluxed under static N₂ for 24 h. The crude product was purified by flash chromatography using a Biotage Isolera instrument (SiO₂, pentane) to afford methyl-3(E)-(4-(2,2-difluorovinyl)phenyl)acrylate (70.8 mg, 63%) as a white solid. The crude product was purified by flash column chromatography (SiO₂, gradient of 0%→70% CH₂Cl₂ in pentane) to afford a white solid. ¹H NMR (500 MHz, CDCl₃): δ 7.66 (d, J=16.0 Hz, 1H), 7.49 (d, J=8.3 Hz, 2H), 7.35 (d, J=8.2 Hz, 2H), 6.43 (d, J=16.0 Hz, 1H), 5.30 (dd, J=26.1, 3.6 Hz, 1H), 3.81 (s, 3H) ppm. ¹³C NMR (126 MHz, CDCl₃): δ 167.40, 156.60 (dd, J=299.9, 290.1 Hz), 144.10, 133.05 (t, J=2.2 Hz), 132.50 (t, J=6.7 Hz), 128.53, 128.00 (dd, J=6.6, 3.6 Hz), 117.77, 82.07 (dd, J=29.7, 13.4 Hz), 51.74 ppm. ¹⁹F NMR (376 MHz, CDCl₃): δ −80.08 (t, J=26.1 Hz), −82.13 (dd, J=26.2, 3.6 Hz) ppm. The NMR spectra are in agreement with those reported in the literature. Observed HRMS (DART-MS): [M+H]+=225.06873, Predicted [M+H]⁺=225.06824.

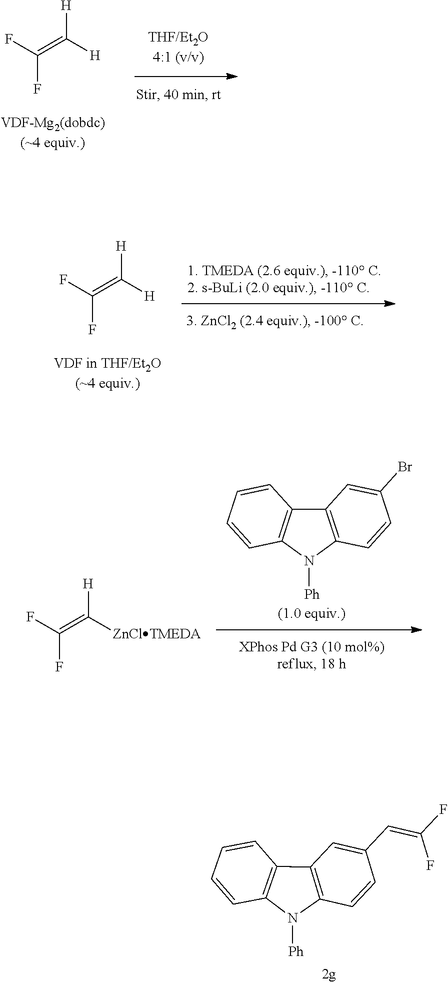

3-(2,2-difluorovinyl)-9-phenyl-9H-carbazole (2g). Prepared according to General Procedure 2. To a 10 mL Schlenk flask containing the 2,2-difluorovinylzinc chloride-TMEDA complex (−280 mg, 1 mmol, 2 equiv. generated in situ), 3-bromo-9-phenyl-9H-carbazole (161.1 mg, 500.0 μmol, 1.000 equiv.) and Xphos Pd G3 (42.4 mg, 50.0 μmol, 10.0 mol %) were quickly added under positive N₂ flow. The reaction was refluxed under static N₂ for 18 h. The crude product was purified by flash chromatography using a Biotage Isolera instrument (SiO₂, hexanes) to afford 3-(2,2-difluorovinyl)-9-phenyl-9H-carbazole (126.7 mg, 83%) as a yellow oil. ¹H NMR (500 MHz, CDCl₃): δ 8.22-8.14 (m, 2H), 7.68-7.61 (m, 2H), 7.61-7.56 (m, 2H), 7.54-7.49 (m, 1H), 7.49-7.46 (m, 2H), 7.45-7.38 (m, 2H), 7.38-7.32 (m, 1H), 5.51 (dd, J=26.5, 3.9 Hz, 1H) ppm. ¹⁹F NMR (376 MHz, CDCl₃): δ −84.73 (dd, J=37.7, 26.4 Hz), −86.50 (dd, J=37.6, 4.0 Hz) ppm. ¹³C NMR (126 MHz, CDCl₃): δ 156.00 (dd, J=296.2, 286.2 Hz), 141.36, 139.92 (t, J=1.8 Hz), 137.60, 130.01, 130.01, 127.65, 127.09, 127.09, 126.37, 125.88 (dd, J=6.0, 3.2 Hz), 123.83, 123.21, 122.11 (t, J=6.1 Hz), 120.48, 120.26, 119.49 (dd, J=6.6, 3.7 Hz), 110.09, 110.03, 82.65 (dd, J=29.0, 13.9 Hz). HRMS (DART-MS): [M+H]⁺=306.10428, Predicted [M+H]⁺=306.10496. IR (neat, cm⁻¹): 3044 (w), 1728 (m), 1627 (w), 1597 (m), 1500 (m), 1486 (w), 1454 (m), 1435 (w), 1360 (m), 1330 (w), 1232 (s), 1215 (w), 1182 (w), 1154 (w), 1135 (w), 1075 (w), 1027 (w), 950 (w), 934 (w), 913 (w), 884 (w), 825 (w), 805 (m), 744 (s), 697 (m), 654 (w), 637 (w), 619 (w), 582 (w), 565 (w), 540 (w).

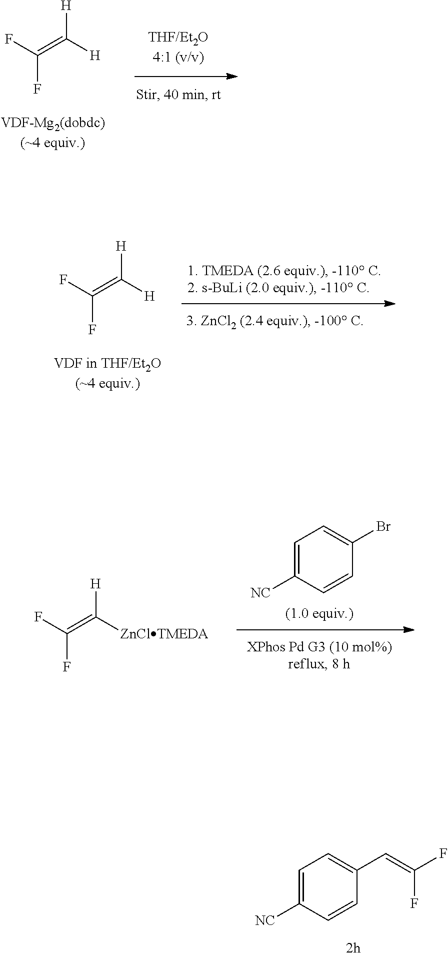

4-(2,2-difluorovinyl)benzonitrile (2h). Prepared according to General Procedure 2. To a 10 mL Schlenk flask containing the 2,2-difluorovinylzinc chloride-TMEDA complex (−280 mg, 1 mmol, 2 equiv. generated in situ), 4-bromobenzonitrile (91.0 mg, 500. Mmol, 1.000 equiv.) and Xphos Pd G3 (42.4 mg, 50.0 μmol, 10.0 mol %) were quickly added under positive N₂ flow. The reaction was refluxed under static N₂ for 8 h. The crude product was purified by flash chromatography using a Biotage Isolera instrument (SiO₂, 0→30% CH₂C₁₂ in hexanes) to afford 4-(2,2-difluorovinyl)benzonitrile (49.5 mg, 60%) as a white solid. ¹H NMR (500 MHz, CDCl₃): δ 7.61 (d, J=8.5 Hz, 2H), 7.42 (d, J=8.5 Hz, 2H), 5.33 (dd, J=25.6, 3.3 Hz, 1H) ppm. ¹³C NMR (126 MHz, CDCl₃): δ 157.08 (dd, J=301.2, 292.2 Hz), 135.45 (dd, J=7.6, 6.4 Hz), 132.55, 128.15 (dd, J=6.9, 3.7 Hz), 118.77, 110.64 (t, J=2.4 Hz), 81.93 (dd, J=30.5, 12.9 Hz) ppm. ¹⁹F NMR (376 MHz, CDCl₃): δ −77.81 (dd, J=25.4, 20.6 Hz), −79.47 (dd, J=20.5, 3.3 Hz). The NMR spectra are in agreement with those reported in the literature. Observed HRMS (DART-MS): [M+H]⁺=166.04622, Predicted [M+H]⁺=166.04236.

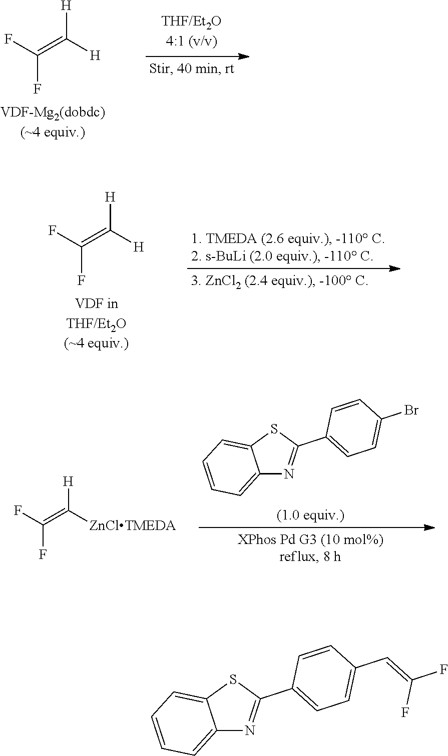

2-(4-(2,2-difluorovinyl)phenyl)benzo[d]thiazole (2i). Prepared according to General Procedure 2. To a 10 mL Schlenk flask containing the 2,2-difluorovinylzinc chloride-TMEDA complex (−280 mg, 1 mmol, 2 equiv. generated in situ), 2-(4-bromophenyl)benzo[d]thiazole (145.1 mg, 500.0 μmol, 1.000 equiv.) and Xphos Pd G3 (42.4 mg, 50.0 μmol, 10.0 mol %) were quickly added under positive N₂ flow. The reaction was refluxed under static N₂ for 8 h. The crude product was purified by flash chromatography using a Biotage Isolera instrument (SiO₂, 0→20% CH₂Cl₂ in Pentane) to afford 2-(4-(2,2-difluorovinyl)phenyl)benzo[d]thiazole (42.4 mg, 31%) as a white solid. ¹H NMR (500 MHz, CDCl₃): δ 8.07 (dd, J=8.7, 7.1 Hz, 3H), 7.92-7.88 (m, 1H), 7.54-7.46 (m, 1H), 7.44 (d, J=8.4 Hz, 2H), 7.41-7.36 (m, 1H), 5.34 (dd, J=26.0, 3.6 Hz, 1H) ppm. ¹³C NMR (126 MHz, CDCl₃): δ 167.45, 156.75 (dd, J=300.1, 290.3 Hz), 154.26, 135.12, 133.25 (t, J=6.7 Hz), 132.25 (t, J=2.3 Hz), 128.15 (dd, J=6.7, 3.6 Hz), 127.89, 126.48, 125.34, 123.32, 121.71, 82.16 (dd, J=29.7, 13.3 Hz). ¹⁹F NMR (376 MHz, CDCl₃): δ −79.73 (t, J=25.8 Hz), −81.77 (dd, J=25.5, 3.6 Hz) ppm. HRMS (DART-MS): [M+H]⁺=274.04733, Predicted [M+H]⁺=274.04573. Melting Point: 93.8-95.8° C. IR (neat, cm⁻¹): 3061 (w), 3032 (w), 2963 (w), 2659 (w), 1947 (w), 1764 (w), 1723 (m). 1607 (w), 1557 (w), 1521 (w), 1483 (m), 1456 (w), 1433 (w), 1415 (w), 1351 (w), 1319 (m), 1250 (m), 1229 (w), 1167 (m), 1093 (w), 1012 (w), 966 (m), 935 (m), 856 (m), 831 (m), 799 (w), 754 (m), 727 (m), 704 (w), 669 (w), 621 (m), 595 (w), 553 (m), 503 (w).

Pd-Catalyzed Heck Coupling of Aryl Halides with TFP-Mg₂(dobdc).

TABLE 12
Optimization of Pd-catalyzed Heck coupling of TFP and electron-
neutral aryl bromides using TFP-Mg₂(dobdc).

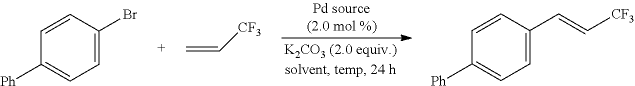

| Entry | Catalyst | Additive | Solvent | Temp | Yieldª |
|---|---|---|---|---|---|
| 1 | Pd(OAc)₂ | — | DMF | 150° C. | 17% |
| 2 | Pd(PPh₃)₂Cl₂ | — | DMF | 150° C. | 47% |
| 3 | DPPF Pd G3 | — | DMF | 150° C. | 88% |
| 4 | DPPF Pd G3 | TBAB | DMF | 150° C. | 78% |
| 5 | DPPF Pd G3 | — | DMSO | 150° C. | 69% |
| 6 | DPPF Pd G3 | — | DMF | 140° C. | 90% |

^{a}Yields determined by ¹⁹F NMR using fluorobenzene as an internal standard. TBAB, tetrabutylammonium bromide; dimethyl sulfoxide, DMSO

Evaluation of Reaction Conditions with Electron-Neutral Aryl Halides (Table 12). In a N₂-filled glovebox, a 15 mL pressure vessel was charged with a magnetic stir bar, 4-bromo-1,1′-biphenyl (116.6 mg, 500.0 μmol, 1.000 equiv.), anhydrous potassium carbonate (K₂CO₃) (138 mg, 1.00 mmol, 2.00 equiv.), Pd catalyst (10.0 μmol, 2.00 mol %), and TFP-Mg₂(dobdc) (350 mg TFP-Mg₂(dobdc), 43 wt % TFP, ˜1.6 mmol, ˜3 equiv.). Anhydrous solvent (5.0 mL) was added, and the vessel was quickly capped. The reaction was heated to the specified temperature and stirred for 24 h in the glovebox. After 24 h, the reaction was allowed to cool to room temperature before fluorobenzene was added (47.1 μL, 500. Mmol). The reaction yields were determined by ¹⁹F NMR using fluorobenzene as an internal standard. The use of DPPF Pd G3 in DMF at 140° C. was found to be optimal.

TABLE 13
Optimization of Pd-catalyzed Heck coupling of TFP and electron-rich
aryl bromides using TFP-Mg₂(dobdc).

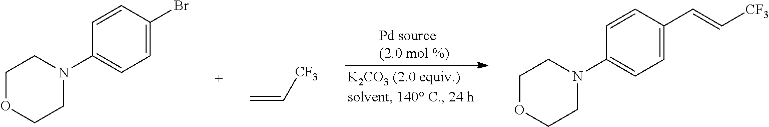

| Entry | Catalyst | Additive (1 equiv.) | Yield^{a} |
|---|---|---|---|
| 1 | DPPF Pd G3 | — | 38% |
| 2 | XPhos Pd G3 | — | 7% |
| 3 | XantPhos Pd G3 | — | 46% |
| 4 | BINAP Pd G3 | — | 22% |
| 5 | XantPhos Pd G3 | — | 24% |
| 6 | XantPhos Pd G3 | TBAB | 60% |
| 7 | XantPhos Pd G3 | TBAB + NaBr | 61% |

ªYields determined by ¹⁹F NMR using fluorobenzene as an internal standard. TBAB, tetrabutylammonium bromide

Evaluation of Reaction Conditions with Electron-Rich Aryl Halides (Table 13). In a N₂-filled glovebox, a 15 mL pressure vessel was charged with a magnetic stir bar, 4-(4-bromophenyl)morpholine (121.1 mg, 500.0 μmol, 1.000 equiv.), anhydrous K₂CO₃ (138 mg, 1.00 mmol, 2.00 equiv.), tetrabutylammonium bromide (TBAB) (161 mg, 500. Mmol, 1.00 equiv.), Pd catalyst (10.0 μmol, 2.00 mol %), and TFP-Mg₂(dobdc) (350 mg TFP-Mg₂(dobdc), 43 wt % TFP, ˜1.6 mmol, ˜3 equiv.). Anhydrous DMF (5.0 mL) was added, and the vessel was quickly capped. The reaction was heated to 140° C. and stirred for 24 h in the glovebox. After 24 h, the reaction was allowed to cool to room temperature before fluorobenzene was added (47.1 μL, 500. Mmol). The reaction yields were determined by ¹⁹F NMR using fluorobenzene as an internal standard. The use of XantPhos Pd G3 in conjunction with TBAB as a phase transfer catalyst was found to be optimal.

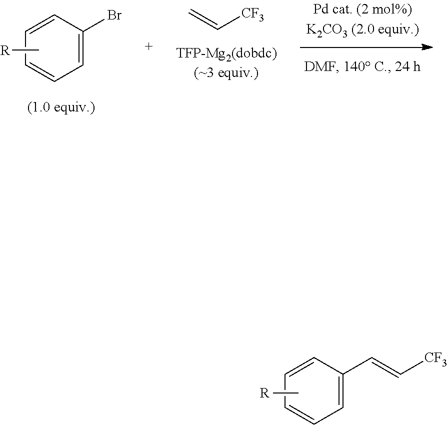

General Procedure 3. A 25 mL Schlenk flask was charged with a magnetic stir bar, aryl bromide (116.6 mg, 500.0 μmol, 1.000 equiv.), anhydrous K₂CO₃ (138 mg, 1.00 mmol, 2.00 equiv.), the specified Pd G3 precatalyst (10.0 μmol, 2.00 mol %), along with TBAB (161 mg, 500. Mmol, 1.00 equiv.) when specified. The flask was evacuated and backfilled with N₂. This process was repeated a total of three times. In a N₂-filled glovebox, a custom-built, solid-addition funnel was loaded with freshly prepared Mg₂(dobdc) dosed with TFP (350 mg TFP-Mg₂(dobdc), 43 wt % TFP, ˜1.6 mmol, ˜3 equiv.). The funnel was sealed and removed from the glovebox. Under positive N₂ pressure, the glass stopper on the reaction flask was quickly replaced with the solid-addition funnel. The funnel was used to dispense the MOF to the reaction under static N₂. Anhydrous DMF (5.0 mL) was added via syringe through the solid-addition funnel to the reaction flask. The solid-addition funnel was sealed. The reaction was heated to 140° C. and stirred for 24 h under static N₂. After 24 h, the reaction mixture was allowed to cool to room temperature and was vacuum-filtered to remove the MOF. The filtrate was diluted with CH₂Cl₂ (50 mL). The mixture was washed with water (5×50 mL). The organic layer was evaporated under reduced pressure and purified by flash chromatography using a Biotage Isolera instrument. The purified product was dried under high vacuum (<100 mTorr) for 12 h prior to characterization.

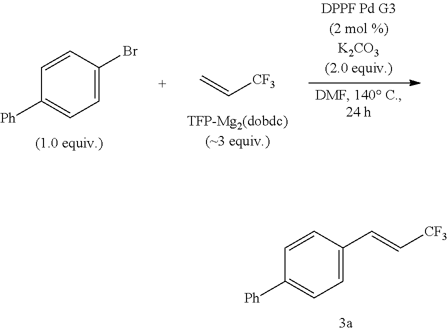

(E)-4-(3,3,3-trifluoroprop-1-en-1-yl)-1,1′-biphenyl (3a). Prepared according to General Procedure 3. To a 10 mL Schlenk flask, 4-bromo-1,1′-biphenyl (116.6 mg, 500.0 μmol, 1.000 equiv.), anhydrous K₂CO₃ (138 mg, 1.00 mmol, 2.00 equiv.), DPPF Pd G3 precatalyst (9.2 mg, 10. Mmol, 2.0 mol %), TFP-Mg₂(dobdc) (350 mg TFP-Mg₂(dobdc), 43 wt % TFP, ˜1.6 mmol, ˜3 equiv.), and anhydrous DMF (5.0 mL) were added and stirred at 140° C. for 24 h. The crude product was purified by flash chromatography using a Biotage Isolera instrument (SiO₂, pentane) to afford (E)-4-(3,3,3-trifluoroprop-1-en-1-yl)-1,1′-biphenyl (83.1 mg, 67%) as a white solid. ¹H NMR (500 MHz, CDCl₃): δ 7.67-7.58 (m, 4H), 7.56-7.51 (m, 2H), 7.50-7.43 (m, 2H), 7.38 (tt, J=7.5 Hz, 1.5 Hz, 1H), 7.20 (dq, J=16.0, 2.2 Hz, 1H), 6.25 (dq, J=16.1, 6.5 Hz, 1H). ¹³C NMR (126 MHz, CDCl₃): δ 142.97, 140.25, 137.35 (q, J=6.8 Hz), 132.49, 129.06, 128.16, 128.00, 127.73, 127.20, 123.81 (q, J=269.6 Hz), 115.86 (q, J=33.8 Hz). ¹⁹F NMR (376 MHz, CDCl₃): δ −63.21 (dd, J=6.5, 2.2 Hz). The NMR spectra are in agreement with those reported in the literature. Observed HRMS (DART-MS): [M+H]⁺=249.0838, Predicted [M+H]⁺=249.08464.

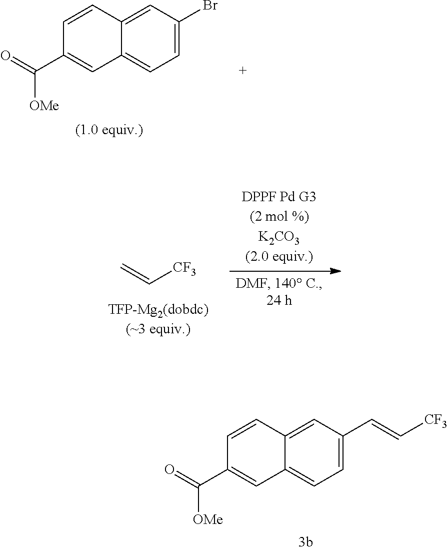

Methyl (E)-6-(3,3,3-trifluoroprop-1-en-1-yl)-2-naphthoate (3b). Prepared according to General Procedure 3. To a 10 mL Schlenk flask, methyl 6-bromo-2-naphthoate (132.6 mg, 500.0 μmol, 1.000 equiv.), anhydrous K₂CO₃ (138 mg, 1.00 mmol, 2.00 equiv.), DPPF Pd G3 precatalyst (9.2 mg, 10. Mmol, 2.0 mol %), TFP-Mg₂(dobdc) (350 mg TFP-Mg₂(dobdc), 43 wt % TFP, ˜1.6 mmol, ˜3 equiv.), and anhydrous DMF (5.0 mL) were added and stirred at 140° C. for 24 h. The crude product was purified by flash chromatography using a Biotage Isolera instrument (SiO₂, gradient of 0%→50% CH₂Cl₂ in pentane) to afford methyl (E)-6-(3,3,3-trifluoroprop-1-en-1-yl)-2-naphthoate (95.3 mg, 68%) as a white solid. ¹H NMR (500 MHz, CDCl₃): δ 8.57 (s, 1H), 8.08 (dd, J=8.7, 1.7 Hz, 1H), 7.93 (d, J=8.6 Hz, 1H), 7.86 (d, J=8.0 Hz, 2H), 7.62 (dd, J=8.6, 1.6 Hz, 1H), 7.30 (d, J=2.4 Hz, 1H), 6.35 (dq, J=16.0, 6.5 Hz, 1H), 3.98 (s, 3H). ¹³C NMR (126 MHz, CDCl₃): δ 167.03, 137.39 (q, J=6.8 Hz), 135.48, 133.19, 130.82, 130.27, 128.72 (dd, J=25.4, 22.5 Hz), 126.36, 124.10, 1233.63 (q, J=271 Hz), 117.37 (q, J=34.0 Hz), 52.48. ¹⁹F NMR (376 MHz, CDCl₃): δ −63.36 (dd, J=6.5, 2.6 Hz). Observed HRMS (DART-MS): [M+H]⁺=281.07394, Predicted [M+H]⁺=281.07447. Melting Point: 126.4-127.2° C. IR (neat, cm⁻¹): 2948 (w), 1716 (m), 1659 (w), 1630 (w), 1478 (w), 1435 (w), 1414 (w), 1393 (w), 1350 (w), 1312 (w), 1296 (w), 1280 (m), 1250 (w), 1206 (m), 1173 (w), 1118 (w), 1094 (m), 993 (w), 979 (m), 959 (w), 914 (w), 875 (w), 832 (w), 804 (m), 783 (w), 768 (w), 750 (m), 681 (w), 644 (w), 568 (w), 523 (w), 507 (w).

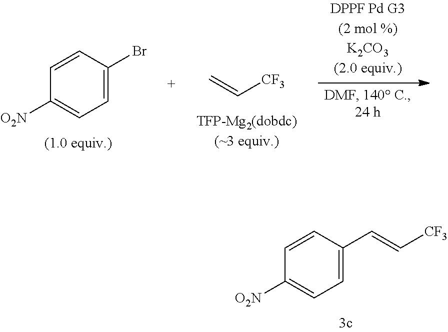

(E)-1-nitro-4-(3,3,3-trifluoroprop-1-en-1-yl)benzene (3c). Prepared according to General Procedure 3. To a 10 mL Schlenk flask, 1-bromo-4-nitrobenzene (101.0 mg, 500.0 μmol, 1.000 equiv.), anhydrous K₂CO₃ (138 mg, 1.00 mmol, 2.00 equiv.), DPPF Pd G3 precatalyst (9.2 mg, 10. Mmol, 2.0 mol %), TFP-Mg₂(dobdc) (350 mg TFP-Mg₂(dobdc), 43 wt % TFP, ˜1.6 mmol, ˜3 equiv.), and anhydrous DMF (5.0 mL) were added and stirred at 140° C. for 24 h. The crude product was purified by flash chromatography using a Biotage Isolera instrument (SiO₂, gradient of 0%→20% CH₂Cl₂ in pentane) to afford (E)-1-nitro-4-(3,3,3-trifluoroprop-1-en-1-yl)benzene (35.7 mg, 33%) as a light yellow solid. ¹H NMR (500 MHz, CDCl₃): δ 8.27 (d, J=8.5 Hz, 2H), 7.63 (d, J=8.5 Hz, 2H), 7.23 (dq, J=16.2, 2.2 Hz, 1H), 6.36 (dq, J=16.2, 6.3 Hz, 1H) ppm. ¹³C NMR (126 MHz, CDCl₃): δ 148.69, 139.61, 135.58 (q, J=6.7 Hz), 128.45, 124.39, 123.00 (q, J=270.06 Hz), 120.24 (q, J=34.5 Hz). ¹⁹F NMR (376 MHz, CDCl₃): δ −63.97 (dd, J=6.4, 2.1 Hz). The NMR spectra are in agreement with those reported in the literature. Observed HRMS (DART-MS): [M+H]⁺=218.04221, Predicted [M+H]⁺=218.03842.

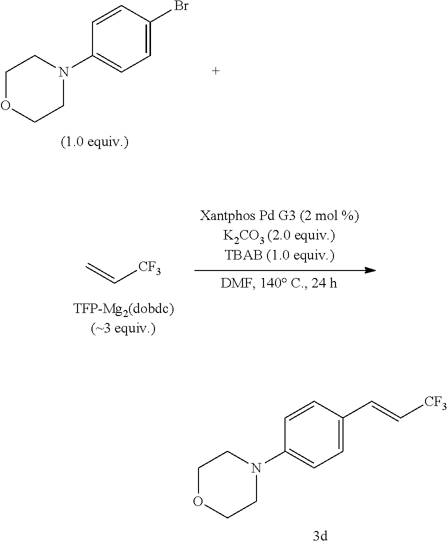

(E)-4-(4-(3,3,3-trifluoroprop-1-en-1-yl)phenyl)morpholine (3d). Prepared according to General Procedure 3. To a 10 mL Schlenk flask, 4-(4-bromophenyl)morpholine (121.1 mg, 500.0 μmol, 1.00 equiv.), anhydrous K₂CO₃ (138 mg, 1.00 mmol, 2.00 equiv.), TBAB (161 mg, 500. Mmol, 1.00 equiv.), XantPhos Pd G3 precatalyst (9.5 mg, 10. Mmol, 2.0 mol %), TFP-Mg₂(dobdc) (350 mg TFP-Mg₂(dobdc), 43 wt % TFP, ˜1.6 mmol, ˜3 equiv.), and anhydrous DMF (5.0 mL) were added and stirred at 140° C. for 24 h. The crude product was purified by flash chromatography using a Biotage Isolera instrument (SiO₂, pentane) to afford (E)-4-(4-(3,3,3-trifluoroprop-1-en-1-yl)phenyl)morpholine (55.4 mg, 43%) as a light tan solid. ¹H NMR (500 MHz, CDCl₃): δ 7.37 (d, 2H), 7.06 (dq, J=16.1, 2.2 Hz, 1H), 6.88 (d, 2H), 6.04 (dq, J=16.1, 6.7 Hz, 1H), 3.86 (d, J=5.0 Hz, 4H), 3.23 (t, J=5.0 Hz, 4H) ppm. ¹³C NMR (126 MHz, CDCl₃): δ 152.41, 137.29 (q, J=6.8 Hz), 128.92, 124.64, 124.24 (q, J=268.2 Hz) 115.03, 112.57 (q, J=33.5 Hz), 66.82, 48.45. ¹⁹F NMR (376 MHz, CDCl₃): δ −62.58 (dd, J=6.5, 2.2 Hz). Observed HRMS (DART-MS): [M+H]⁺=258.10941, Predicted [M+H]⁺=258.10610. Melting Point: 102.7-104.4° C. IR (neat, cm⁻¹): 2953 (w), 2861 (w), 2830 (w), 1658 (w), 1606 (m), 1568 (w), 1517 (w), 1449 (w), 1429 (w), 1382 (w), 1353 (w), 1312 (m), 1263 (m), 1223 (w), 1193 (m), 1106 (s), 1068 (w), 1050 (w), 981 (m), 947 (w), 936 (w), 921 (m), 874 (w), 846 (w), 812 (m), 733 (w), 616 (m), 583 (w), 528 (w).

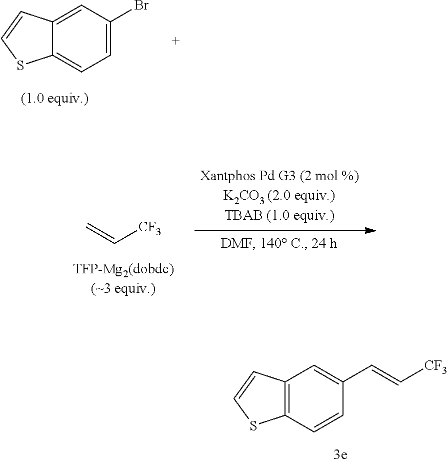

(E)-5-(3,3,3-trifluoroprop-1-en-1-yl)benzo[b]thiophene (3e). Prepared according to General Procedure 3. To a 10 mL Schlenk flask, 5-bromobenzo[b]thiophene (106.5 mg, 500.0 μmol, 1.000 equiv.), anhydrous K₂CO₃ (138 mg, 1.00 mmol, 2.00 equiv.), TBAB (161 mg, 500. Mmol, 1.00 equiv.), XantPhos Pd G3 precatalyst (9.5 mg, 10. Mmol, 2.0 mol %), TFP-Mg₂(dobdc) (350 mg TFP-Mg₂(dobdc), 43 wt % TFP, ˜1.6 mmol, ˜3 equiv.), and anhydrous DMF (5.0 mL) were added and stirred at 140° C. for 24 h. The crude product was purified by flash chromatography using a Biotage Isolera instrument (SiO₂, pentane) to afford (E)-5-(3,3,3-trifluoroprop-1-en-1-yl)benzo[b]thiophene (62.6 mg, 55%) as a white solid. ¹H NMR (500 MHz, CDCl₃): δ 7.94-7.87 (m, 2H), 7.53 (d, J=5.4 Hz, 1H), 7.48 (dd, J=8.4, 1.8 Hz, 1H), 7.38 (dd, J=5.4, 0.8 Hz, 1H), 7.30 (dq, J=16.2, 2.3 Hz, 1H), 6.30 (dq, J=16.1, 6.5 Hz, 1H). ¹³C NMR (126 MHz, CDCl₃): δ 141.37, 140.12, 137.98 (q, J=6.8 Hz), 129.91, 127.85, 124.10, 123.67 (q, J=268.4 Hz), 123.65, 123.09, 122.75, 115.50 (q, J=33.8 Hz) ppm. ¹⁹F NMR (376 MHz, CDCl₃): δ −63.05 (dd, J=6.6, 2.2 Hz). Observed HRMS (DART-MS): [M+H]⁺=229.02455, Predicted [M+H]⁺=229.02541. Melting Point: 112.7-114.4° C. IR (neat, cm⁻¹): 2922 (w), 2852 (w), 1660 (m), 1440 (w), 1417 (w), 1332 (w), 1315 (w), 1275 (m), 1261 (w), 1231 (w), 1168 (w), 1084 (m), 1048 (w), 972 (m), 952 (w), 935 (w), 900 (w), 870 (w), 833 (w), 806 (m), 778 (w), 755 (w), 699 (m), 687 (w), 637 (m), 535 (w), 512 (w).

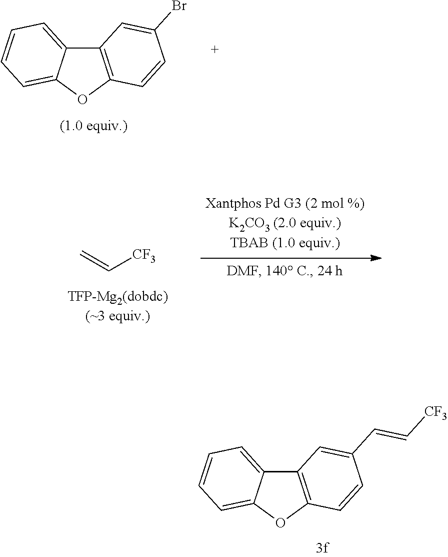

(E)-2-(3,3,3-trifluoroprop-1-en-1-yl)dibenzo[b,d]furan 3f. Prepared according to General Procedure 3. To a 10 mL Schlenk flask, 2-bromodibenzo[b,d]furan (123.5 mg, 500.0 μmol, 1.000 equiv.), anhydrous K₂CO₃ (138 mg, 1.00 mmol, 2.00 equiv.), TBAB (161 mg, 500. Mmol, 1.00 equiv.), XantPhos Pd G3 precatalyst (9.5 mg, 10.0 μmol, 2.00 mol %), TFP-Mg₂(dobdc) (350 mg TFP-Mg₂(dobdc), 43 wt % TFP, ˜1.6 mmol, ˜3 equiv.), and anhydrous DMF (5.0 mL) were added and stirred at 140° C. for 24 h. The crude product was purified by flash chromatography using a Biotage Isolera instrument (SiO₂, pentane) to afford (E)-2-(3,3,3-trifluoroprop-1-en-1-yl)dibenzo[b,d]furan (53.8 mg, 41%) as a white solid. ¹H NMR (400 MHz, CDCl₃): δ 8.05 (s, 1H), 7.97 (d, J=1.3 Hz, 1H), 7.62-7.54 (m, 3H), 7.50 (ddd, J=8.4, 7.2, 1.3 Hz, 1H), 7.38 (ddd, J=7.5, 7.4, 1.1 Hz, 1H), 7.31 (dq, 1H), 6.27 (dq, J=16.1, 6.5 Hz, 1H) ppm. ¹³C NMR (126 MHz, CDCl₃): δ 157.21, 156.88, 137.77 (q, J=6.8 Hz), 128.56, 127.97, 126.78, 125.11, 123.9 (q, J=269.64) 123.75, 123.30, 120.93, 120.15, 115.15 (q, J=33.8 Hz), 112.31, 112.06 ppm. ¹⁹F NMR (376 MHz, CDCl₃): δ −63.02 (dd, J=6.4, 2.3 Hz) ppm. Observed HRMS (DART-MS): [M+H]⁺=263.06293, Predicted [M+H]⁺=263.06390. Melting Point: 125.6-127.6° C. IR (neat, cm⁻¹): 2920 (w), 1665 (w), 1599 (w), 1586 (w), 1475 (w), 1452 (w), 1351 (w), 1320 (w), 1306 (w), 1291 (w), 1269 (m), 1246 (w), 1202 (w), 1121 (w), 1090 (m), 1023 (w), 974 (m), 947 (w), 938 (w), 896 (w), 870 (w), 841 (w), 812 (m), 769 (w), 752 (m), 732 (w), 700 (w), 689 (w), 639 (w), 620 (w), 549 (w), 525 (w).

Trifluoromethylation of (Hetero)Arenes using TFMI-Mg₂(dobdc).

TABLE 14
Optimization of trifluoromethylation of uracil using TFMI-Mg₂(dobdc)

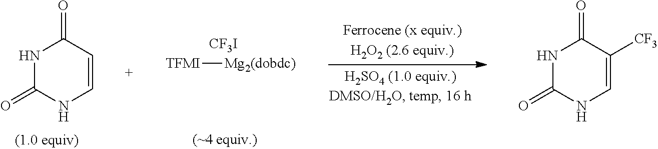

| Entry | Fc (x equiv.) | Temp | Yield^{a} |
|---|---|---|---|
| 1 | 0.30 | 50° C. | 15% |
| 2 | 1.0 | 100° C. | 1% |
| 3 | 0.30 | 100° C. | 31% |

ªYields determined by ¹⁹F NMR using fluorobenzene as an internal standard.

Evaluation of Reaction Conditions (Table S14). Pyrimidine-2,4(1H,3H)-dione (56.0 mg, 500. Mmol, 1.00 equiv.), ferrocene (0.30 equiv. or 1.00 equiv.), TFMI-Mg₂(dobdc) (617 mg TFMI-Mg₂(dobdc), 64 wt % TFMI, ˜2.0 mmol, ˜4 equiv.), a 30% aqueous solution of H₂O₂(0.10 mL, 1.3 mmol, 2.6 equiv.), a solution of H₂SO₄ (0.5 M in DMSO, 1.0 mL, 0.5 mmol, 1.0 equiv.), and dimethyl sulfoxide (DMSO) (0.8 mL) were all added to a 10 mL Schlenk flask. The mixture was stirred for 16 h at the specified temperature. The use of 0.30 equiv. of ferrocene at 100° C. was found to be optimal.

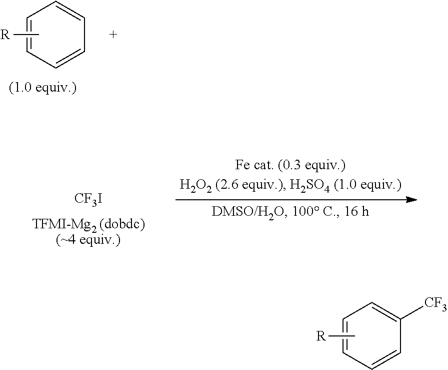

General Procedure 4. A 10 mL Schlenk flask was charged with a stir bar, (hetero)arene (500. Mmol, 1.00 equiv.), and the specified Fe catalyst (150. Mmol, 0.300 equiv.). In a N₂-filled glovebox, a custom-built, solid-addition funnel was loaded with freshly prepared Mg₂(dobdc) dosed with TFMI (617 mg TFMI-Mg₂(dobdc), 64 wt % TFMI, ˜2.0 mmol, ˜4 equiv.). The funnel was sealed and removed from the glovebox. Under positive N₂ pressure, the glass stopper on the reaction flask was quickly replaced with the solid-addition funnel. Under static N₂, the funnel was used to dispense the MOF to the reaction flask. Under static N₂, DMSO (1.0 mL) was added via syringe through the solid-addition funnel. The solid-addition funnel was closed. Through the sidearm of the Schlenk flask, a 30% aqueous solution of H₂O₂(0.10 mL, 1.3 mmol, 2.6 equiv.), a solution of H₂SO₄ (0.5 M in DMSO, 1.0 mL, 0.5 mmol, 1.0 equiv.), and DMSO (0.8 mL) were all added sequentially. The mixture was stirred at the indicated temperature for 16 h. After 16 h, the reaction mixture was allowed to cool to room temperature and was vacuum-filtered to remove solids. The crude reaction mixture was purified by flash chromatography using a Biotage Isolera instrument. The purified product was dried under high vacuum (<100 mTorr) for 12 h prior to characterization.

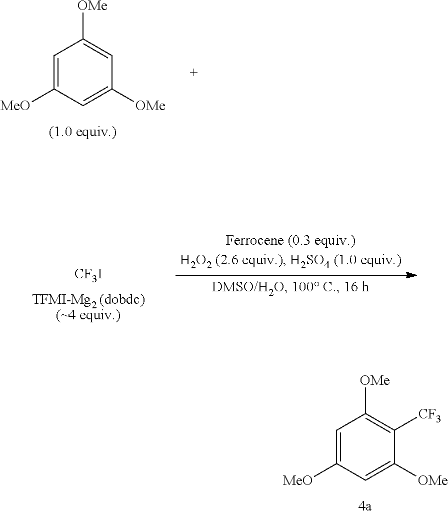 [SMILES: COc1cc(OC)cc(OC)c1 ; "CF3I" ; COc1cc(OC)c(C(F)(F)F)c(OC)c1]

1,3,5-trimethoxy-2-(trifluoromethyl)benzene (4a). Prepared according to General Procedure 4. A 10 mL Schlenk flask was charged with 1,3,5-trimethoxybenzene (84.1 mg, 500. Mmol, 1.00 equiv.), ferrocene (27.9 mg, 150. Mmol, 0.300 equiv.), TFMI-Mg₂(dobdc) (617 mg TFMI-Mg₂(dobdc), 64 wt % TFMI, ˜2.0 mmol, ˜4 equiv.), a 30% aqueous solution of H₂O₂(0.10 mL, 1.3 mmol, 2.6 equiv.), a solution of H₂SO₄ (0.5 M in DMSO, 1.0 mL, 0.5 mmol, 1.0 equiv.), and DMSO (0.8 mL). The mixture was stirred at 100° C. for 16 h. The crude reaction mixture was purified by flash chromatography using a Biotage Isolera instrument (SiO₂, gradient of 0%→10% ethyl acetate in hexanes) to afford 1,3,5-trimethoxy-2-(trifluoromethyl)benzene (31.2 mg, 26%) as a white solid. ¹H NMR (400 MHz, CDCl₃): δ 6.13 (s, 2H), 3.84 (s, 9H) ppm. ¹³C NMR (126 MHz, CDCl₃): δ 163.65, 160.54, 124.49 (q, J=273.3 Hz), 100.48 (q, J=30.0 Hz), 91.35, 56.36, 55.49 ppm. ¹⁹F NMR (376 MHz, CDCl₃): δ −54.18 ppm. The NMR spectra are in agreement with those reported in the literature. Observed HRMS (DART-MS): [M+H]⁺=237.07268, Predicted [M+H]⁺=237.06938.

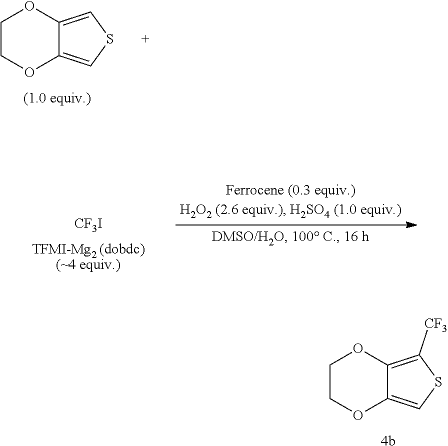 [SMILES: c1scc2c1OCCO2 ; "CF3I" ; FC(F)(F)c1scc2c1OCCO2]

3,4-ethylenedioxy-2-(trifluoromethyl)thiophene (4b). Prepared according to General Procedure 4. A 10 mL Schlenk flask was charged with 3,4-ethylenedioxythiophene (71.1 mg, 500. Mmol, 1.00 equiv.), ferrocene (27.9 mg, 150. Mmol, 0.300 equiv.), TFMI-Mg₂(dobdc) (617 mg TFMI-Mg₂(dobdc), 64 wt % TFMI, ˜2.0 mmol, ˜4 equiv.), a 30% aqueous solution of H₂O₂(0.10 mL, 1.3 mmol, 2.6 equiv.), a solution of H₂SO₄ (0.5 M in DMSO, 1.0 mL, 0.5 mmol, 1.0 equiv.), and DMSO (0.8 mL). The mixture was stirred at 100° C. for 16 h. The crude reaction mixture was purified by flash chromatography using a Biotage Isolera instrument (SiO₂, gradient of 0%→5% CH₂Cl₂ in pentane) to afford 3,4-ethylenedioxy-2-(trifluoromethyl)thiophene (27.5 mg, 26%) as a clear liquid. ¹H NMR (400 MHz, CDCl₃): δ 6.49 (s, 1H), 4.34-4.27 (m, 2H), 4.27-4.20 (m, 2H) ppm. ¹³C NMR (126 MHz, CDCl₃): δ 142.11 (q, J=3.3 Hz), 141.46, 122.37 (q, J=268.0 Hz), 104.58 (q, J=39.2 Hz), 102.25 (q, J=1.9 Hz), 65.08, 64.40 ppm. ¹⁹F NMR (376 MHz, CDCl₃): δ −55.37 ppm. The NMR spectra are in agreement with those reported in the literature. Observed HRMS (DART-MS): [M+H]⁺=210.99960, Predicted [M+H]⁺=210.99959.

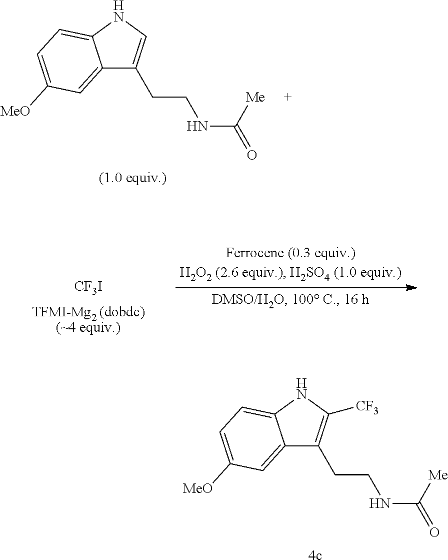 [SMILES: COc1ccc2[nH]cc(CCNC(C)=O)c2c1 ; "CF3I" ; COc1ccc2[nH]c(C(F)(F)F)c(CCNC(C)=O)c2c1]

Prepared according to General Procedure 4. A 10 mL Schlenk flask was charged with N-(2-(5-methoxy-1H-indol-3-yl)ethyl)acetamide (116.1 mg, 500.0 μmol, 1.000 equiv.), ferrocene (27.9 mg, 150. Mmol, 0.300 equiv.), TFMI-Mg₂(dobdc) (617 mg TFMI-Mg₂(dobdc), 64 wt % TFMI, ˜2.0 mmol, ˜4 equiv.), a 30% aqueous solution of H₂O₂(0.10 mL, 1.3 mmol, 2.6 equiv.), a solution of H₂SO₄ (0.5 M in DMSO, 1.0 mL, 0.5 mmol, 1.0 equiv.), and DMSO (0.8 mL). The mixture was stirred at 100° C. for 16 h. The crude reaction mixture was purified by flash chromatography using a Biotage Isolera instrument (SiO₂, gradient of 0%+10% MeOH in CH₂Cl₂) to afford N-(2-(5-methoxy-2-(trifluoromethyl)-1H-indol-3-yl)ethyl)acetamide (9.5 mg, 6%) as a white solid. ¹H NMR (400 MHz, CDCl₃): δ 8.67 (s, 1H), 7.31 (d, J=8.8 Hz, 1H), 7.09 (s, 1H), 6.99 (dd, J=8.8, 2.3 Hz, 1H), 5.62 (s, 1H), 3.85 (s, 3H), 3.55 (q, J=6.4 Hz, 2H), 3.08 (t, J=6.7 Hz, 2H), 1.93 (s, 3H) ppm. ¹³C NMR (126 MHz, CDCl₃): δ 170.49, 155.00, 130.58, 127.96, 122.82 (q, J=336.8 Hz), 122.09 (q, J=286.6 Hz), 116.32, 114.75 (q, J=2.8 Hz), 113.02, 100.74, 55.94, 40.12, 24.13, 23.43 ppm. ¹⁹F NMR (376 MHz, CDCl₃): δ −57.93 ppm. The NMR spectra are in agreement with those reported in the literature. Observed HRMS (DART-MS): [M+H]⁺=301.11475, Predicted [M+H]⁺=301.11192.

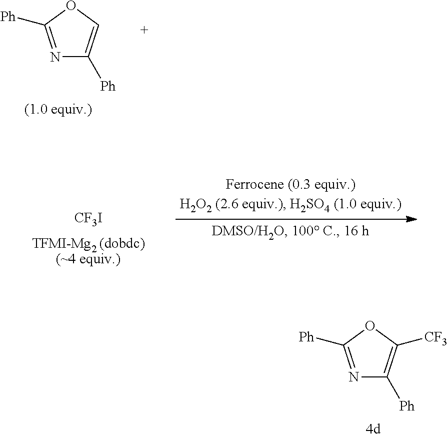 [SMILES: c1ccc(-c2coc(-c3ccccc3)n2)cc1 ; "CF3I" ; FC(F)(F)c1oc(-c2ccccc2)nc1-c1ccccc1]

2,4-diphenyl-5-(trifluoromethyl)oxazole (4d). Prepared according to General Procedure 4. A 10 mL Schlenk flask was charged with 2,4-diphenyloxazole (110.6 mg, 500.0 μmol, 1.000 equiv.), ferrocene (27.9 mg, 150. Mmol, 0.300 equiv.), TFMI-Mg₂(dobdc) (617 mg TFMI-Mg₂(dobdc), 64 wt % TFMI, ˜2.0 mmol, ˜4 equiv.), a 30% aqueous solution of H₂O₂(0.10 mL, 1.3 mmol, 2.6 equiv.), a solution of H₂SO₄ (0.5 M in DMSO, 1.0 mL, 0.5 mmol, 1.0 equiv.), and DMSO (0.8 mL). The mixture was stirred at 50° C. for 16 h. The crude reaction mixture was purified by flash chromatography using a Biotage Isolera instrument (SiO₂, gradient of 0%→35% CH₂Cl₂ in pentane) to afford 2,4-diphenyl-5-(trifluoromethyl)oxazole (48.1 mg, 33%) as a white solid. ¹H NMR (400 MHz, CDCl₃): δ 8.16 (dd, J=7.7, 1.9 Hz, 2H), 7.83-7.75 (m, 2H), 7.59-7.41 (m, 6H) ppm. ¹³C NMR (126 MHz, CDCl₃): δ 161.82, 142.62 (q, J=2.6 Hz), 133.66 (q, J=42.7 Hz), 131.77, 129.73, 129.48, 129.09, 128.74, 128.64 (q, J=1.9 Hz), 127.27, 126.19, 119.96 (q, J=267.9 Hz) pm. ¹⁹F NMR (376 MHz, CDCl₃): δ −60.12 ppm. The NMR spectra are in agreement with those reported in the literature. Observed HRMS (DART-MS): [M+H]⁺=290.07745, Predicted [M+H]⁺=290.07480.

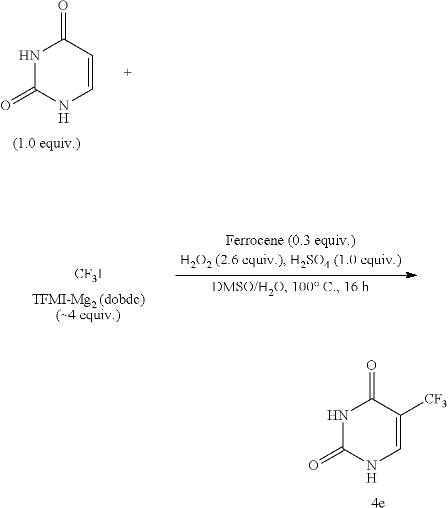 [SMILES: O=c1cc[nH]c(=O)[nH]1 ; "CF3I" ; O=c1[nH]cc(C(F)(F)F)c(=O)[nH]1]

5-(trifluoromethyl)pyrimidine-2,4(1H,3H)-dione (4e). Prepared according to General Procedure 4. A 10 mL Schlenk flask was charged with pyrimidine-2,4(1H,3H)-dione (56.0 mg, 500. Mmol, 1.00 equiv.), ferrocene (27.9 mg, 150. Mmol, 0.300 equiv.), TFMI-Mg₂(dobdc) (617 mg TFMI-Mg₂(dobdc), 64 wt % TFMI, ˜2.0 mmol, ˜4 equiv.), a 30% aqueous solution of H₂O₂(0.10 mL, 1.3 mmol, 2.6 equiv.), a solution of H₂SO₄ (0.5 M in DMSO, 1.0 mL, 0.5 mmol, 1.0 equiv.), and DMSO (0.8 mL). The mixture was stirred at 100° C. for 16 h. The crude reaction mixture was purified by flash chromatography using a Biotage Isolera instrument (SiO₂, gradient of 0%→7% MeOH in CH₂Cl₂) to afford 5-(trifluoromethyl)pyrimidine-2,4(1H,3H)-dione (17.4 mg, 19%) as a white solid. ¹H NMR (400 MHz, DMSO-d6): δ 11.61 (d, J=16.2 Hz, 2H), 8.03 (s, 1H) ppm. ¹³C NMR (126 MHz, DMSO-d6): δ 159.90 (d, J=13.7 Hz), 150.55 (d, J=12.1 Hz), 144.19 (q, J=5.7 Hz), 122.91 (q, J=268.6 Hz), 101.78 (q, J=31.7 Hz) ppm. ¹⁹F NMR (376 MHz, DMSO-d6): δ −61.57 ppm. The NMR spectra are in agreement with those reported in the literature. Observed HRMS (DART-MS): [M+H]⁺=181.01201, Predicted [M+H]⁺=181.01802.

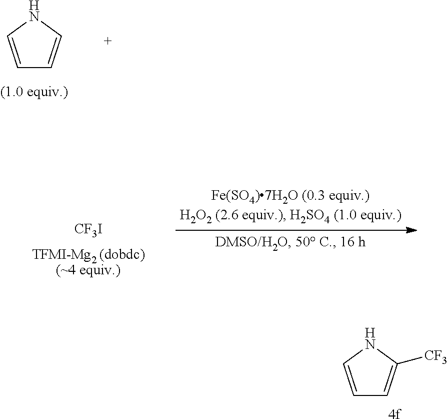 [SMILES: c1cc[nH]c1 ; "CF3I" ; FC(F)(F)c1ccc[nH]1]

2-(trifluoromethyl)-1H-pyrrole (4f). Prepared according to General Procedure 4. A 10 mL Schlenk flask was charged with 1H-pyrrole (33.4p L, 500. Mmol, 1.00 equiv.), iron(II) sulfate heptahydrate (41.7 mg, 150. Mmol, 0.300 equiv.), TFMI-Mg₂(dobdc) (617 mg TFMI-Mg₂(dobdc), 64 wt % TFMI, ˜2.0 mmol, ˜4 equiv.), a 30% aqueous solution of H₂O₂ (0.10 mL, 1.3 mmol, 2.6 equiv.), a solution of H₂SO₄ (0.5 M in DMSO, 1.0 mL, 0.5 mmol, 1.0 equiv.), and DMSO (0.8 mL). The mixture was stirred at 50° C. for 16 h. The compound was not isolated due to its volatility. ¹⁹F NMR (376 MHz, CDCl₃): δ −58.66 ppm. Observed HRMS (DART-MS): [M+H]⁺=136.02095, Predicted [M+H]⁺=136.03294. The NMR spectra are in agreement with those reported in the literature.

Pd-Catalyzed Defluorinative Coupling of Arylboronic Acids with HFP-Mg₂(dobdc).

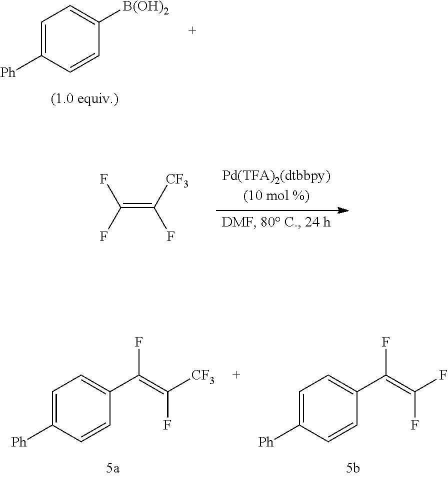 [SMILES: OB(O)c1ccc(-c2ccccc2)cc1 ; FC(F)=C(F)C(F)(F)F ; F/C(=C(/F)C(F)(F)F)c1ccc(-c2ccccc2)cc1 ; FC(F)=C(F)c1ccc(-c2ccccc2)cc1]

(E)-4-(perfluoroprop-1-en-1-yl)-1,1′-biphenyl (5a) and (Z)-4-(perfluoroprop-1-en-1-yl)-1,1′-biphenyl (5b). Prepared according to a modified version of General Procedure 1. A 10 mL Schlenk flask was charged with a magnetic stir bar, 4-biphenylboronic acid (49.5 mg, 250. Mmol, 1.00 equiv.), and Pd(TFA)₂(dtbbpy) (15.0 mg, 25.0 μmol, 10.0 mol %). The flask was evacuated and backfilled with N₂. This process was repeated a total of three times. In a N₂-filled glovebox, a custom-built, solid-addition funnel was loaded with freshly prepared Mg₂(dobdc) dosed with HFP (466 mg HFP-Mg₂(dobdc), 46 wt % HFP, ˜1.4 mmol, ˜5.7 equiv.). The funnel was sealed and removed from the glovebox. Under positive N₂ pressure, the glass stopper on the reaction flask was quickly replaced with the solid-addition funnel. The funnel was used to dispense the MOF to the reaction under static N₂. Anhydrous DMF (5.0 mL) was added via syringe through the solid-addition funnel to the reaction flask. The solid-addition funnel was sealed. The reaction was heated to 80° C. and stirred for 24 h under static N₂. After 24 h, the reaction mixture was allowed to cool to room temperature and was vacuum-filtered to remove the MOF. The crude reaction mixture was purified by flash chromatography using a Biotage Isolera instrument (SiO₂, pentane) to afford 4-(perfluoroprop-1-en-1-yl)-1,1′-biphenyl (4.1 mg, 6%, E:Z 1:0.9) as a white solid. The purified product was dried under high vacuum (<100 mTorr) for 12 h prior to characterization. ¹H NMR (500 MHz, CDCl₃): δ 7.78 (d, J=8.3 Hz, 1H), 7.74-7.66 (m, 2H), 7.62 (t, J=7.8 Hz, 2H), 7.55 (d, J=8.0 Hz, 1H), 7.48 (td, J=7.7, 2.2 Hz, 2H), 7.41 (t, J=7.9 Hz, 1H) ppm. ¹³C NMR (126 MHz, CDCl₃): δ 144.39 (dd, J=62.2, 2.3 Hz), 139.82, 129.53 (d, J=1.7 Hz), 129.15 (d, J=2.0 Hz), 128.39, 127.56 (d, J=2.0 Hz), 127.34 (d, J=6.0 Hz), 127.13 (t, J=8.1 Hz), 126.00 (dd, J=23.7, 6.5 Hz), 125.47, 125.29 ppm. ¹⁹F NMR (376 MHz, CDCl₃): δ −65.51 (dd, J=13.0, 8.1 Hz), −66.80 (dd, J=22.0, 10.7 Hz), −107.57,-110.99 (m), −146.65 (dd, J=131.2, 22.0 Hz), −154.15 (dd, J=13.0, 9.5 Hz), −169.21 (dd, J=131.2, 10.6 Hz) ppm. Observed HRMS (DART-MS): [M+H]⁺=285.06381, Predicted [M+H]⁺=285.06580. Melting Point: 65.6-67.4° C.; 74.0-75.8° C. IR (neat, cm⁻¹): 1720 (w), 1699 (w), 1607 (w), 1557 (w), 1487 (w), 1451 (w), 1407 (w), 1358 (m), 1290 (m), 1201 (w), 1135 (s), 1091 (w), 1056 (m), 1018 (w), 1005 (w), 925 (m), 867 (w), 843 (s), 766 (s), 727 (m), 709 (w), 690 (s), 644 (w), 633 (w), 584 (w), 562 (w), 545 (w), 510 (w), 1582 (w), 1521 (w), 1316 (w), 1164 (w), 1176 (w), 806 (w).

PXRD Analyses of Mg₂(dobdc) after Successful Reactions. After completion of the relevant General Procedure, the Mg₂(dobdc)—which was recovered from the reaction mixture by vacuum filtration—was transferred to a 20 mL scintillation vial filled with 10 mL of MeOH. The vial was placed in an aluminum heat block. The heat block was transferred to a dry block heater, which was then heated to 60° C. and allowed to stand at this temperature for 24 h. At this time, the heterogeneous mixture was allowed to cool to room temperature, the solvent was decanted, and fresh MeOH (10 mL) was added. The vial was returned to the heat block that was heated to 60° C. This process was repeated for a total of 3 MeOH soaks. The sample was vacuum filtered again and characterized by PXRD as shown in FIG. 22. The MOF was found to decompose after General Procedure 4.

Stability Tests for VDF-Mg₂(dobdc).

TABLE 15
Performance of VDF-Mg₂(dobdc) after air exposure

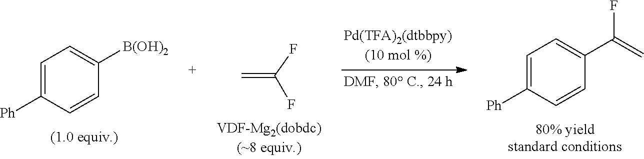

| Entry | Exposure of VDF-Mg₂(dobdc) to Air | Yield |
|---|---|---|
| 1 | None | 80% |
| 2 | Add in air | 82% |
| 3 | Store in air at rt o/n, then add in air | 65% |

Evaluation of the Performance of VDF-Mg₂(dobdc) After Exposure to Air (Table 15). A 15 mL pressure vessel was charged with a magnetic stir bar, 4-biphenylboronic acid (49.5 mg, 250. Mmol, 1.00 equiv.), and Pd(TFA)₂(dtbbpy) (15.0 mg, 25.0 μmol, 10.0 mol %). VDF-Mg₂(dobdc) was added to the pressure vessel in the specified manner for each entry (see details for handling below). Anhydrous DMF (5.0 mL) was added to the pressure vessel and the vessel was quickly sealed. The reaction was heated to 80° C. and stirred for 24 h. The reaction yields were determined by ¹⁹F NMR using fluorobenzene as an internal standard.

Entry 1. Prepared according to General Procedure 1. The 4-(1-fluorovinyl)-1,1′-biphenyl product was obtained in 80% yield.

Entry 2. In the glovebox, freshly prepared VDF-Mg₂(dobdc) (375 mg VDF-Mg₂(dobdc), 34 wt % VDF, −2.0 mmol, −8.0 equiv.) was transferred to a 20 mL vial. The vial was sealed and removed from the glovebox. The VDF-Mg₂(dobdc) was then quickly transferred to the reaction vessel. The 4-(1-fluorovinyl)-1,1′-biphenyl product was obtained in 82% yield.

Entry 3. In the glovebox, freshly prepared VDF-Mg₂(dobdc) (375 mg VDF-Mg₂(dobdc), 34 wt % VDF, −2.0 mmol, −8.0 equiv.) was transferred to a 20 mL vial. The vial was sealed and removed from the glovebox. The vial was stored at RT on the benchtop overnight. Then, VDF-Mg₂(dobdc) was quickly transferred to the reaction vessel. The 4-(1-fluorovinyl)-1,1′-biphenyl product was obtained in 65% yield.

TABLE 16
Performance of VDF-Mg₂(dobdc) after storage under different conditions.

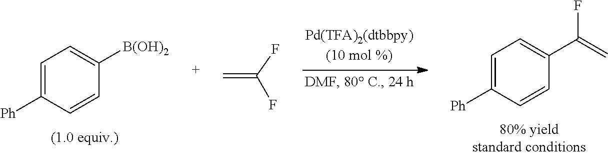

| Days | Glovebox Freezer (−30 ° C.) | Lab Freezer (−20 ° C.) | Desiccator (RT) |
|---|---|---|---|
| 1 | 83% | 89% | 82% |
| 3 | 88% | 83% | 72% |
| 7 | 82% | 80% | 47% |

Evaluation of the Performance of VDF-Mg₂(dobdc) After Storage Under Different Conditions (Table 16). A 15 mL pressure vessel was charged with a magnetic stir bar, 4-biphenylboronic acid (49.5 mg, 250. Mmol, 1.00 equiv.), and Pd(TFA)₂(dtbbpy) (15.0 mg, 25.0 μmol, 10.0 mol %). After storage under different conditions (glovebox freezer, lab freezer, or desiccator) for the specified time (1, 3, or 7 days), VDF-Mg₂(dobdc) (375 mg VDF-Mg₂(dobdc), 34 wt % VDF, −2 mmol, −8 equiv.) was quickly weighed and added to the pressure vessel in the specified manner for each condition (see details for storage and handling below). DMF (5.0 mL) was added to the pressure vessel and the vessel was quickly sealed. The reaction was heated to 80° C. and stirred for 24 h. The reaction yields were determined by ¹⁹F NMR using fluorobenzene as an internal standard.

Glovebox Freezer (−30° C.). In the glovebox, freshly prepared VDF-Mg₂(dobdc) (1.6 g) was weighed and transferred to a 20 mL vial. The vial was stored in the glovebox freezer (−30° C.). After the specified time of storage (1, 3, or 7 days), VDF-Mg₂(dobdc) (375 mg VDF-Mg₂(dobdc), 34 wt % VDF, −2 mmol, −8 equiv.) was removed from the glovebox freezer, weighed in the glovebox, and transferred to a 20 mL vial. The vial was sealed and removed from the glovebox. The VDF-Mg₂(dobdc) was then quickly transferred to the reaction vessel in air.

Lab Freezer (−20° C.). In the glovebox, freshly prepared VDF-Mg₂(dobdc) (1.6 g) was weighed and transferred to a 20 mL vial. The vial was sealed and removed from the glovebox. The vial was stored in the lab freezer (−20° C.) in air. After the specified time of storage (1, 3, or 7 days), VDF-Mg₂(dobdc) (375 mg VDF-Mg₂(dobdc), 34 wt % VDF, −2 mmol, −8 equiv.) was removed from the freezer and quickly weighed in air. The VDF-Mg₂(dobdc) was then quickly transferred to the reaction vessel in air. The remaining VDF-Mg₂(dobdc) in the vial that was not used for the reaction was quickly returned to the lab freezer for storage.

Desiccator (RT). In the glovebox, freshly prepared VDF-Mg₂(dobdc) (1.6 g) was weighed and transferred to a 20 mL vial. The vial was sealed and removed from the glovebox. The vial was stored in a desiccator at RT. After the specified time of storage (1, 3, or 7 days), VDF-Mg₂(dobdc) (375 mg VDF-Mg₂(dobdc), 34 wt % VDF, −2 mmol, −8 equiv.) was removed from the desiccator and quickly weighed in air. The VDF-Mg₂(dobdc) was then quickly transferred to the reaction vessel in air. The remaining VDF-Mg₂(dobdc) in the vial that was not used for the reaction was quickly returned to the desiccator for storage.

Preparation of Wax Capsules. The wax capsules were prepared according to a modified previous literature procedure. A 300 mL beaker was charged with a stir bar and paraffin wax. The wax was heated to 62° C. while stirring under air, resulting in approximately 200 mL of molten wax. A room temperature metal rod (1 mm diameter) was dipped into the molten wax (to a depth of 3.0 cm) 10 times. The resulting wax coating was allowed to cool to room temperature and dry for 10 minutes. The wax coating was removed from the metal rod slowly to give a hollow wax case. If any cracks developed upon removal from the metal rod, they were repaired by dipping the hollow paraffin case back into the molten wax three times.

To prepare small wax cylinders to cap the hollow wax cases, the bottom of a 24/40 Suba-Seal rubber septum (Sigma Aldrich part number: Z124656) was filled with molten wax. The wax cylinders were allowed to dry for 20 minutes before they were removed from the rubber septum. The wax cylinder was shaped to fit the hollow wax case using a metal spatula.

The hollow wax cases and wax cylinders were transferred into a nitrogen filled glovebox. In the glovebox, the hollow case was packed with freshly prepared VDF-Mg₂(dobdc) (375 mg VDF-Mg₂(dobdc), 34 wt % VDF, −2 mmol). Next, the wax cylinder was packed on top of the VDF-Mg₂(dobdc) in the hollow case. The open end of the capsule was then melted shut using a metal spatula that had been warmed on a hot plate. The sealed capsule was removed from the glovebox and was stored on the benchtop until use.

TABLE 17
Performance of VDF-Mg₂(dobdc) after storage in a wax capsule

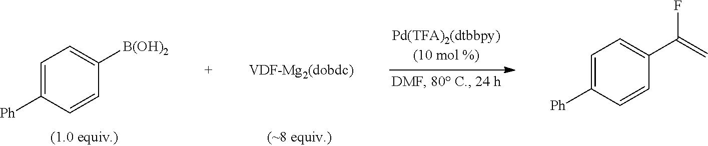

| Days | Yield |
|---|---|
| 1 | 80% |
| 7 | 72% |

Evaluation of the Performance of VDF-Mg₂(dobdc) After Storage in a Wax Capsule (Table 17). A wax capsule was packed with VDF-Mg₂(dobdc) (375 mg VDF-Mg₂(dobdc), 34 wt % VDF, −2 mmol, −8 equiv.) and sealed according to the Preparation of Wax Capsules described above. The capsule containing VDF-Mg₂(dobdc) was stored on the benchtop at RT for the specified amount of time prior to use. A 15 mL pressure vessel was charged with a magnetic stir bar, 4-biphenylboronic acid (49.5 mg, 250. Mmol, 1.00 equiv.), Pd(TFA)₂(dtbbpy) (15.0 mg, 25.0 μmol, 10.0 mol %), and the capsule containing VDF-Mg₂(dobdc). DMF (5.0 mL) was added to the pressure vessel, and the vessel was sealed. The mixture was sonicated for 30 minutes to break open the wax capsule and release the VDF-Mg₂(dobdc). The reaction was heated to 80° C. and stirred for 24 h. The reaction was allowed to cool down to room temperature. The layer of wax that formed above the reaction mixture was broken up with a metal spatula. The wax and MOF were removed via vacuum filtration. The reaction yields were determined by ¹⁹F NMR using fluorobenzene as an internal standard.

TABLE 18
Controlled release of VDF-Mg₂(dobdc) from wax capsule
into DMF over time

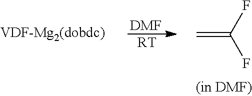

| | | | | | | | | 70 min |
|---|---|---|---|---|---|---|---|---|
| | | 1 | 3 | 5 | 10 | 20 | 60 | (10 min |
| Time | 30 s | min | min | min | min | min | min | sonication) |
| mmol | 0 | 0 | 0 | 0 | 0 | 0 | 0 | 0.33 |
| VDF | | | | | | | | |
| in | | | | | | | | |
| DMF | | | | | | | | |

Evaluation of Release of VDF-Mg₂(dobdc) from a Wax Capsule to DMF Over Time (Table 18). A wax capsule was packed with VDF-Mg₂(dobdc) (190 mg VDF-Mg₂(dobdc), 34 wt % VDF, −1 mmol, −1 equiv.) and sealed according to the Preparation of Wax Capsules described above. The capsule containing VDF-Mg₂(dobdc) was stored on the benchtop at RT for 4 hours prior to use. Meanwhile, eight NMR tubes were sealed with rubber septa (Fisher Scientific, part number: 16800180) and evacuated and refilled with nitrogen three times. Under active nitrogen, anhydrous DMF (450 μL) was syringed into each NMR tube. The tubes were left under active nitrogen until the reaction was dispensed to the tube. A 10 mL Schlenk flask equipped with a stir bar was flame dried under vacuum. The Schlenk flask was allowed to cool under vacuum. A wax capsule containing VDF-Mg₂(dobdc) (190 mg VDF-Mg₂(dobdc), 34 wt % VDF, −1 mmol, −1 equiv.) was added to the Schlenk flask under active nitrogen flow. The Schlenk flask was sealed with a septum. Through the side-arm of the Schlenk flask, anhydrous DMF (8.0 mL) and fluorobenzene (94.2 μL, 1.00 mmol) were added. Aliquots (50 μL) of the reaction mixture were removed via a syringe and dispensed to the NMR tubes at specified time intervals. Sixty minutes after suspending the wax capsule containing VDF-Mg₂(dobdc) in DMF, the reaction mixture was sonicated for 10 minutes to break open the wax capsule and release VDF-Mg₂(dobdc) to DMF. A final aliquot (50 μL) of the reaction mixture was removed via syringe and dispensed to an NMR tube. The VDF delivery was quantified before and after the capsule was broken by ¹⁹F NMR using fluorobenzene as an internal standard.

Control Experiment with Balloon of VDF.

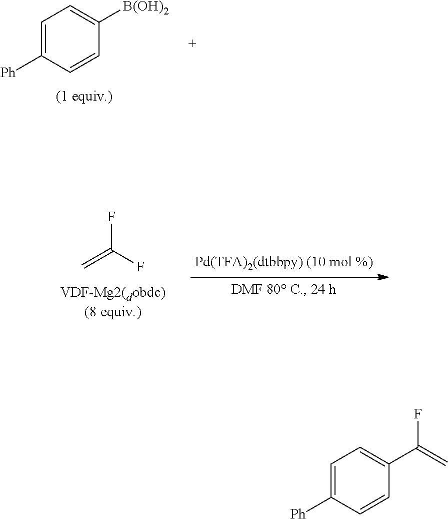

A 10 mL Schlenk flask was charged with a magnetic stir bar, 4-biphenylboronic acid (49.5 mg, 250. Mmol, 1.00 equiv.), and Pd(TFA)₂(dtbbpy) (15.0 mg, 25.0 μmol, 10.0 mol %). The flask was evacuated and backfilled with N₂. This process was repeated a total of three times. Under positive N₂ pressure, the glass stopper on the reaction flask was quickly replaced with a rubber septum. Anhydrous DMF (5.0 mL) was added via syringe through septum into the reaction flask. A balloon filled with VDF (˜48 mL, ˜2 mmol, ˜8 equiv.) was prepared and added to the reaction setup via a needle. The reaction mixture was heated to 80° C. using a silicone oil bath and stirred for 24 h under static N₂. After 24 h, the reaction was allowed to cool before fluorobenzene was added (47.1 μL, 500. Mmol). The 4-(1-fluorovinyl)-1,1′-biphenyl was obtained in a 67% yield by ¹⁹F NMR.

### Example 2

The following is an example of methods of storage and delivery of gaseous chemical reactant(s) in a chemical process using metal-organic frameworks (MOFs), methods of making and uses of MOFs and gaseous chemical reactant-loaded (gas-loaded) MOF(s).

Preparation of CO—Mg₂(dobdc). CO—Mg₂(dobdc) was prepared using a carbon monoxide (CO) cylinder following the general procedure for using Mg₂(dobdc) to deliver gaseous reagents. An oven-baked 10 mL round-bottom flask and a Schlenk adapter were cycled into a N₂-filled glovebox. In the glovebox, activated Mg₂(dobdc) was loaded into the round-bottom flask. The flask was sealed with the Schlenk adapter and removed from the glovebox. The flask containing Mg₂(dobdc) was connected to a Schlenk line and the CO cylinder through a three-way valve. The three-way valve was used to pull vacuum on the regulator of the CO cylinder and to reactivate the Mg₂(dobdc) under high vacuum (<100 mTorr) while ramping the temperature to 300° C. (1° C./min) using a sand bath. The flask was allowed to stand at 300° C. under high vacuum (<100 mTorr) overnight. After activation, the flask was allowed to cool to room temperature under vacuum. Once cool, the joints of the flask containing the Mg₂(dobdc) were secured with copper wire, and the three-way valve was closed to the Schlenk line. The freshly activated sample of Mg₂(dobdc) was immediately dosed with approximately 1 bar of CO at approximately 0° C. (in an ice-water bath) for 40 min. After 40 min, the flask was closed with the Schlenk adapter under approximately 1 bar of CO, and the CO—Mg₂(dobdc) was immediately brought into a N₂-filled glovebox. Once in the glovebox, the flask was placed in the freezer at −30° C. for 10 min. After 10 min, the freshly dosed CO—Mg₂(dobdc) was weighed and transferred to the reaction mixture.

Pd-Catalyzed Carbonylative Suzuki-Miyaura Coupling of Carbon Monoxide, Aryl Bromide, and Arylboronic Acid using CO—Mg₂(dobdc).

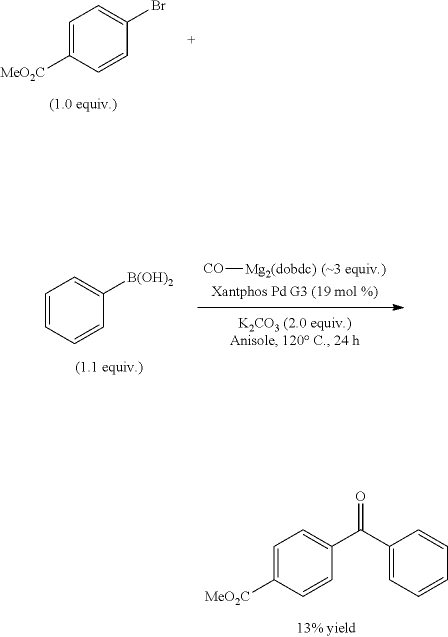

Methyl 4-Benzoylbenzoate. In a N₂-filled glovebox, a 15 mL pressure vessel was charged with a magnetic stir bar, phenylboronic acid (67.0 mg, 0.55 mmol, 1.1 equiv.), methyl 4-bromobenzoate (107.5 mg, 0.5 mmol, 1.0 equiv.) Xantphos Pd G3 (90.0 mg, 0.095 mmol, 19 mol %), K₂CO₃ (138.2 mg, 1.0 mmol, 2.0 equiv.), and freshly prepared CO—Mg₂(dobdc) (273 mg, 15 wt % CO, ˜1.5 mmol, ˜3 equiv.). Anhydrous anisole (5 mL) was added to the pressure vessel and the vessel was closed quickly. The reaction was heated to 120° C. and stirred for 24 h in the glovebox. After 24 h, the reaction was allowed to cool to RT, and the vessel was removed from the glovebox. The MOF was removed by vacuum filtration. The crude product was purified by flash chromatography using a Biotage Isolera instrument (0%→2% ethyl acetate in hexanes) to afford methyl 4-benzoylbenzoate (15.6 mg, 13% yield) as a white solid. The purified product was dried under high vacuum (<100 mTorr) for 12 h. ¹H NMR (500 MHz, CDCl₃) δ 8.15 (d, J=8.5 Hz, 2H), 7.87-7.79 (m, 4H), 7.62 (t, J=7.5 Hz, 1H), 7.50 (t, J=7.8 Hz, 2H), 3.97 (s, 3H). The ¹H NMR spectrum was in agreement with that reported in the reference.¹ Observed HRMS (DART-MS): [M+H]⁺=241.08527, Predicted [M+H]⁺=241.08200.

Although the present disclosure has been described with respect to one or more particular embodiments and/or examples, it will be understood that other embodiments and/or examples of the present disclosure may be made without departing from the scope of the present disclosure.

1. A method of providing one or more gaseous chemical reactant(s) in a chemical process, the method comprising:
forming a chemical reaction mixture comprising:
one or more metal-organic framework(s) (MOF(s)) comprising one or more gaseous chemical reactant(s), wherein each individual MOF comprises the following structure and/or formula 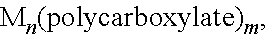
wherein M is a metal ion, wherein n is 1, 2, or, and wherein m is 1, 2, or 3 and wherein the gaseous chemical reactant(s) is/are material(s) which exist in a gas phase under the reaction conditions of the chemical reaction mixture; and
optionally, one or more additional chemical reactant(s);
wherein a chemical reaction of the chemical process occurs between at least a portion of, substantially all of, or all of the gaseous chemical reactant(s) and at least a portion of, substantially all of, or all of the additional chemical reactant(s), if present.

2. The method of claim 1, wherein M is independently at each occurrence chosen from Mg ions, Ni ions, Co ions, Cu ions, Fe ions, Mₙ ions, Cd ions, Zn ions, Al ions, and Zr ions.

3. The method of claim 1, wherein the polycarboxylate(s) is independently at each occurrence chosen from 2,5-dioxido-1,4-benzenedicarboxylate (dobdc⁴⁻), 2,4-dioxidobenzene-1,3-dicarboxylate (m-dobdc⁴⁻), benzene-1,3,5-tricarboxylate (btc³⁻), and 1,4-benzenedicarboxylate (bdc²⁻), and 2-amino-1,4-benzenedicarboxylate (NH₂-bdc²⁻).

4. The method of claim 1, wherein the ratio of n/m is from about 1:3 to about 3:1.

5. The method of claim 1, wherein the MOF(s), independently at each occurrence, each comprise the following structure and/or formula: Mg₂(dobdc), Mn₂(dobdc), Fe₂(dobdc), Co₂(dobdc), Ni₂(dobdc), Cu₂(dobdc), Zn₂(dobdc), Mg₂(m-dobdc), Ni₂(m-dobdc), Cu₃(btc)₂, Fe₃O (OH)(btc)₂, or Al₃O(OH)(NH₂-bdc)₃.

6. The method of claim 1, wherein at least a portion or all of the MOF(s) are sequestered in an inert material configured to expose at least a portion of, substantially all, or all the MOFs to the reaction mixture under the reaction conditions or at least a portion or all of the MOF(s) are present in an inert container configured to release at least a portion of, substantially all, or all the MOF(s) into the reaction mixture.

7. The method of claim 1, wherein the gaseous chemical reactant(s) is/are independently at each occurrence chosen from hazardous gaseous chemical reactant(s), sensitive gaseous chemical reactant(s), environmentally harmful gaseous chemical reactant(s), and any combination thereof.

8. The method of claim 1, wherein the gaseous chemical reactant(s) is/are chosen from halogenated gaseous chemical reactant(s), oxocarbon gaseous chemical reactant(s), halogen gaseous chemical reactant(s), sulfur gaseous chemical reactant(s), and any combination thereof.

9. The method of claim 1, wherein the additional chemical reactant(s) is/are chosen from Negishi coupling reactant(s), Heck coupling reactant(s), trifluoromethylation reactant(s), defluorinative cross-coupling reactant(s), carbonylative Suzuki coupling reactant(s), difluoromethylation reactant(s), copper-catalyzed borylation reactant(s), hydroarylation reactant(s), aminocarbonylation reactant(s), olefin metathesis reactant(s), fluoroalkylation reactant(s), deoxyfluorination reactant(s), deoxyfluoroalkoxylation reactant(s), fluoroalkylthiolation reactant(s), fluoroalkylselenation reactant(s), fluorovinylation reactant(s), fluoroalkynylation reactant(s), and pentafluorosulfanylation reactant(s).

10. The method of claim 1, wherein the chemical reaction mixture comprises one or more solvent(s).

11. The method of claim 1, wherein the gas-loaded MOF(s), at a temperature of about −196° C. to about 55° C., comprise(s), on average, from about 0.1 millimole (mmol) gaseous chemical reactant(s)/gram gas-loaded MOF(s) to about 10 mmol gaseous chemical reactant(s)/gram gas-loaded MOF(s).

12. The method of claim 1, the method further comprising, prior to the occurrence of the chemical reaction, releasing at least a portion of, substantially all of, or all of the gaseous chemical reactant(s) from the gas-loaded MOF(s) into the chemical reaction mixture.

13. The method of claim 12, wherein the releasing at least a portion of, substantially all of, or all of the gaseous chemical reactant(s) from the gas-loaded MOF(s) into the chemical reaction mixture is achieved by increasing the chemical reaction mixture temperature, reducing the chemical reaction mixture pressure, adding a coordinating solvent to displace the gas, irradiation of the reaction mixture with light, sonication of the reaction mixture, mechanical grinding of the reaction mixture, or any combination thereof.

14. The method of claim 1, the method further comprising, prior to forming the chemical reaction mixture:
forming the gas-loaded MOF(s); and
optionally, maintaining the gas-loaded MOF(s) under inert and/or anhydrous conditions.

15. The method of claim 14, wherein the forming the gas-loaded MOF(s) comprises:
optionally, activating the MOF(s);
forming a MOF gas-loading reaction mixture comprising:
the gaseous chemical reactant(s); and
the MOF(s),
wherein the gas-loaded MOF(s) is/are formed, and
optionally, isolating and/or activating the gas-loaded MOF(s).

16. The method of claim 14, the method further comprising, prior to forming the gas-loaded MOF(s), forming the MOF(s).

17. The method of claim 1, the method further comprising, after the gas-loaded MOF(s) has/have released a portion of, substantially all of, or all of the gaseous chemical reactant(s) into the chemical reaction mixture and/or has/have reacted with at least a portion of, substantially all of, or all of the additional chemical reactant(s) in the chemical reaction mixture, wherein spent gas-loaded MOF(s) are formed, isolating and/or activating the spent gas-loaded MOF(s), wherein recycled MOF(s) is/are formed.

18. A method of making one or more metal-organic framework(s) (MOF(s)), wherein each individual MOF comprises (or has) the following formula and/or structure:
Mₙ(polycarboxylate)ₘ,
wherein M is a metal ion independently at each occurrence chosen from Mg ions, Mₙ ions, Co ions, Ni ions, Cu ions, Zn ions, Fe ions, and Al ions, wherein n is 1, 2, or 3, and wherein m is 1, 2, or 3,
the method comprising:
forming a first MOF reaction mixture comprising:
one or more compound(s) independently comprising one or more of the metal ion(s);
one or more polycarboxylic acid(s) and/or salt(s) thereof; and
one or more basic solvent(s),
wherein the reaction mixture comprises 0.1 mol/liter (M) or greater of: the compound(s) comprising the metal ion(s); the polycarboxylic acid(s) and/or the salt(s) thereof, or both; and
heating the reaction mixture, wherein one or more first solid(s) is/are formed;
optionally, isolating the first solid(s);
forming a second MOF reaction mixture comprising:
the first solid(s); and
one or more basic solvent(s),
optionally, wherein the second MOF reaction mixture is maintained under inert and/or anhydrous conditions,
wherein the one or more MOF(s) is/are formed; and
optionally, isolating and/or activating the MOF(s).

19. The method of claim 18, wherein the metal compound(s) comprise magnesium salt(s), nickel salt(s), manganese salt(s), iron salt(s), cobalt salt(s), copper salt(s), zinc salt(s), aluminum salt(s), or a hydrate thereof, or any combination thereof.

20. The method of claim 18, wherein the polycarboxylic acid(s) is/are independently at each occurrence chosen from 2,5-dihydroxyterephthalic acid (H₄dobdc), 1,3,5-benzenetricarboxylic acid (H₃btc), 4,6-dihydroxyisophthalic acid (m-H₄dobdc), and 2-aminoterephthalic acid (NH₂—H₂bdc), and partially or completely deprotonated structural analogs thereof.

21. The method of claim 18, wherein the first MOF reaction mixture comprise from about 1 equivalent(s) (eq) to about 10 eq of polycarboxylic acid(s), polycarboxylate salt(s), or any combination thereof.

22. The method of claim 18, wherein the first MOF reaction mixture is held at a temperature of from about −150 degrees Celsius (° C.) to about 200° C.
